(19) United States
(12) Patent Application Publication
(10) Pub. No.: US 2018/0224433 A1
(43) Pub. Date: Aug. 9, 2018

(54) TECHNIQUES AND SYSTEMS FOR INJECTION AND/OR CONNECTION OF ELECTRICAL DEVICES

(71) Applicant: President and Fellows of Harvard College, Cambridge, MA (US)

(72) Inventors: Charles M. Lieber, Lexington, MA (US); Guosong Hong, Somerville, MA (US); Tian-Ming Fu, Ashburn, VA (US); Jinlin Huang, Somerville, MA (US); Tao Zhou, Somerville, MA (US)

(73) Assignee: President and Fellows of Harvard College, Cambridge, MA (US)

(21) Appl. No.: 15/749,617

(22) PCT Filed: Aug. 4, 2016
(86) PCT No.: PCT/US2016/045587

§ 371 Date: Feb. 1, 2018

Related U.S. Application Data
(60) Provisional application No. 62/201,006, filed on Aug. 4, 2015.
(60) Provisional application No. 62/209,255, filed on Aug. 24, 2015.

(51) Int. Cl. G01N 33/53; A61N 1/05; G01N 33/543

(52) U.S. Cl. CPC G01N 33/5302; G01N 33/5438; G01N 33/5308; A61N 1/0531

(57) ABSTRACT

The present invention generally relates to nanoscale wires, nanoscale sensing elements, and/or injectable devices. In some embodiments, the present invention is directed to electronic devices that can be injected or inserted into soft matter, such as biological tissue or polymeric matrixes. For example, the device may be passed through a tube into the medium. To avoid or minimize crumpling, the device may exit the tube at substantially the same rate that the tube is withdrawn from the medium. Other components, such as fluids or cells, may also be injected or inserted. In addition, in some cases, the device, after insertion or injection, may be connected to an external electrical circuit, for example, by printing a conductive path on a medium or on a flexible substrate. The path may be printed using conductive inks, e.g., containing carbon nanotubes or other suitable materials. Other embodiments are generally directed to systems and methods of making, using, or promoting such devices, kits involving such devices, and the like.

### RELATED APPLICATIONS

This application claims the benefit of U.S. Provisional Patent Application Ser. No. 62/201,006, filed Aug. 4, 2015, entitled “Syringe Injectable Electronics: Precise Targeted Delivery with Quantitative Input/Output,” by Lieber, et al.; and U.S. Provisional Patent Application Ser. No. 62/209,255, filed Aug. 24, 2015, entitled “Techniques and Systems for Injection and/or Connection of Electrical Devices,” by Lieber, et al. Each of these is incorporated herein by reference in its entirety.

### GOVERNMENT FUNDING

This invention was made with government support under FA9550-14-1-0136 awarded by the Air Force Office of Scientific Research. The government has certain rights in the invention.

### FIELD

The present invention generally relates to nanoscale wires, nanoscale sensing elements and/or injectable devices.

### BACKGROUND

Recent efforts in coupling electronics and tissues have focused on flexible, stretchable planar arrays that conform to tissue surfaces, or implantable microfabricated probes. Syringe-injectable mesh electronics with tissue-like mechanical properties and open macroporous structures is an emerging paradigm for mapping and modulating brain activity. Flexible macroporous structures have exhibited minimal non-invasiveness or the promotion of attractive interactions with neurons. These same structural features also pose challenges for precise targeted delivery in specific brain regions and quantitative input/output (I/O) connectivity needed for reliable electrical measurements.

### SUMMARY

The present invention generally relates to nanoscale wires, nanoscale sensing elements, and/or injectable devices. The subject matter of the present invention involves, in some cases, interrelated products, alternative solutions to a particular problem, and/or a plurality of different uses of one or more systems and/or articles.

In one aspect, the present invention is generally directed to a method. In one set of embodiments, the method includes acts of inserting a tube comprising a device comprising one or more nanoscale sensing elements into a medium; and withdrawing the tube from the medium while urging the device out of the tube. In some cases, the rate of withdrawal of the tube from the medium is substantially equal to the rate that the device is urged out of the tube.

The method, in another set of embodiments, is generally directed to inserting a tube comprising a device comprising one or more nano scale sensing elements into a medium, and removing the tube from the medium, without substantially altering the position of the device relative to the medium.

According to yet another set of embodiments, the method includes acts of inserting a device comprising one or more nanoscale sensing elements into a medium, connecting the device to an electrical interface by printing a conductive path directly onto the surface of the medium, where the conductive path is in electrical communication with the one or more nanoscale sensing elements; and covering at least a portion of the conductive path with an insulating material.

In another aspect, the present invention encompasses methods of making one or more of the embodiments described herein, for example, a device comprising one or more nanoscale wires and/or one or more nanoscale sensing elements. The device may be injectable in some cases. In still another aspect, the present invention encompasses methods of using one or more of the embodiments described herein, for example, a device comprising one or more nanoscale wires and/or one or more nanoscale sensing elements. The device may be injectable in some cases.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIGS. 1A-1D illustrate general schemes of controlled injection of devices into a mouse and quantitative connections using conductive ink printing, in accordance with one set of embodiments;

FIGS. 2A-2B illustrate controlled delivery of devices into soft materials (in this case, hydrogel), in another set of embodiments;

FIGS. 3A-3D illustrate “blind” injection of devices into biological systems (in this case, ex vivo and in vivo rodent brains), in yet another set of embodiments;

FIGS. 4A-4D illustrate conductive ink printing, in another set of embodiments;

FIGS. 5A-5B illustrate the structure of an injectable device, in yet another set of embodiments;

FIGS. 6A-6B illustrate injection at various rates;

FIGS. 7A-7C illustrate injection at different angles, in yet another set of embodiments;

FIG. 8 schematically illustrates a cross-section of one embodiment of the invention;

FIGS. 9A-9B illustrates a syringe-injectable mesh electronic device, in another embodiment of the invention;

FIGS. 10A-10E illustrates recordings using a device in accordance with yet another embodiment of the invention;

FIGS. 11A-11F illustrates tracking of neurons, in still another embodiment of the invention;

FIGS. 12A-12G illustrates multi-site and multifunctional mesh electronics, in another embodiment of the invention;

FIGS. 13A-13B illustrates brain aging, in yet another embodiment of the invention; and

FIGS. 14A-14D illustrates brain recordings, in still another embodiment of the invention.

### DETAILED DESCRIPTION

The present invention generally relates to nanoscale wires, nanoscale sensing elements, and/or injectable devices. In some embodiments, the present invention is directed to electronic devices that can be injected or inserted into soft matter, such as biological tissue or polymeric matrixes. For example, the device may be passed through a tube into the medium. To avoid or minimize crumpling, the device may exit the tube at substantially the same rate that the tube is withdrawn from the medium. Other components, such as fluids or cells, may also be injected or inserted. In addition, in some cases, the device, after insertion or injection, may be connected to an external electrical circuit, for example, by printing a conductive path on a medium or on a flexible substrate. The path may be printed using conductive inks, e.g., containing carbon nanotubes or other suitable materials. Other embodiments are generally directed to systems and methods of making, using, or promoting such devices, kits involving such devices, and the like.

One aspect of the present invention is generally directed to a device for insertion or injection into a tissue (e.g., biological tissue), or other matter, including soft matter. The device may be inserted at any suitable angle. The tissue may be in vitro or in vivo (i.e., the device may be injected into a living subject). In some cases, soft matter is matter that exhibits some viscoelasticity, e.g., the matter can undergo deformation, and may exhibit viscous and/or elastic characteristics while undergoing deformation. Examples of soft matter include, but are not limited to, polymers, gels, or other materials having viscoelastic properties. The device can be fully or partially inserted into the tissue or other matter. The device may be used to determine a property of the tissue or other matter, and/or provide an electrical signal to the tissue or other matter. This may be achieved using one or more nanoscale sensing elements on the device. In some cases, at least one of the nanoscale sensing elements is a nanoscale wire, such as a silicon nanowire. In certain embodiments, a device comprising nanoscale sensing elements may be inserted into an electrically-active tissue, such as the heart or the brain, and the nanoscale sensing elements may be used to determine electrical properties of the tissue, e.g., action potentials or other electrical activity. Additional non-limiting examples of tissue include the nerves (e.g., the spinal cord) or the eyes (e.g., within the retina). In some cases, the device is relatively porous to allow cells, etc. to grow or migrate into the device, for example, neurons may grow into the device. This may be useful, for example, for long-term applications, for example, where the device is to be inserted and used for days, weeks, months, or years within the tissue. For example, neurons or cardiac cells may be able to grow around and/or into the device while it is inserted into the brain or the heart, e.g., over extended periods of time.

In some embodiments, a device may be formed from one or more polymeric constructs and/or metal leads. In some cases, the device is relatively small and may include components such as nanoscale sensing elements (e.g., nanoscale wires). The device may also be flexible and/or have a relatively open structure, e.g., an open porosity of at least about 30%, or other porosities as discussed herein. For instance, the device may be formed from a plurality of nanoscale sensing elements, connected by polymeric constructs and/or metal leads, forming a relatively large or open network, which can then be rolled to form a cylindrical or other 3-dimensional structure that is to be injected into a subject. In some cases, the nanoscale sensing elements may be distributed about the device, e.g., in three dimensions, thereby allowing determining properties and/or stimulation of a tissue, etc. in three-dimensions. The device can also be connected to an external electrical system, e.g., to facilitate use of the device. Polymeric constructs, metal leads, nanoscale sensing elements, the structure of the device, and various properties of the devices are all discussed in additional detail below.

In addition, it should be understood that while the discussion herein refers, in some instances, to nanoscale sensing elements, not all embodiments of the invention are limited to only nanoscale sensing elements. For example, a device may comprises nanoscale sensing elements and other nanoscale elements (such as nanoscale wires) that are not used for sensing (for example, they may be used for processing, transmission of information, or other suitable functions). Also, devices containing nanoscale wires (but not necessarily containing any nanoscale sensing elements) are also contemplated in other embodiments of the invention, e.g., for insertion into a subject or other soft matter, such as biological tissue or polymeric matrixes, or other embodiments as described herein. Thus, the descriptions herein with respect to nanoscale sensing elements should be understood as being by way of example only, rather than as limiting the scope of the invention.

In certain aspects, a device as discussed herein may be positioned in a tube, such as a metal tube. The device may be shaped such that it is cylindrical or curved, and/or the device may be compressed to fit inside the tube, although the device may be able to expand after exiting the tube, e.g., as discussed herein. The tube may be formed out of any suitable material. For instance, the tube may comprise stainless steel. The tube may also be other materials in other embodiments. For example, the tube may be plastic, or the tube may be glass. The tube may be a needle or form part of a syringe, or the tube may be form part of an injector device, such as a microinjector. In some cases, the tube is cylindrical, although the tube may be noncylindrical in other cases. For instance, the tube may be tapered or beveled in some embodiments. In some cases, the tube is hollow. In some cases, the tube has a circular cross-section. However, in other cases, the tube may not have a circular cross-section. For example, the tube may have a square or rectangular cross-section, or the tube may have an open cross-section, e.g., having a “U”-shaped cross section. The tube may have any suitable inner diameter. For instance, the tube may have an inner diameter of less than about 1 mm, less than about 800 micrometers, less than about 600 micrometers, less than about 500 micrometers, less than about 400 micrometers, less than about 300 micrometers, less than about 200 micrometers, less than about 100 micrometers, less than about 80 micrometers, less than about 60 micrometers, less than about 50 micrometers, etc.

The device may pass through the tube using any suitable method. The device may fully pass through the tube, or the device may only partially pass through the tube such that a portion of the device remains within the tube. For instance, the device may be fully or partially expelled or urged from the tube using suitable forces, pressures, mechanisms, or apparatuses. For instance, in one set of embodiments, the device may be expelled using a microinjection device. In another embodiment, the device may be manually expelled, e.g., by pushing the plunger of a syringe. In some cases, fluids (liquids or gases) may be added to the tube to expel the device. For instance, water, saline, or air may be added to the tube to cause the device to be expelled therefrom. In some cases, for example, a pump or other fluid source (e.g., a spigot or a tank) may be used to introduce fluid into the tube to expel the device. For instance, a pump may pump fluid into the tube (or through tubing or other fluidic channels) into the tube to cause the device to be expelled therefrom (e.g., partially or fully). The device may be injected at a controlled rate and/or with controllable position, for example, by controlling the pressure or flow rate of fluid from the pump. In some cases, the tube may be inserted into a target such that the device is expelled directly into the target. For example, the tube may be inserted into a subject, e.g., into the tissue of a subject, such as those described herein. In another embodiment, the tube may be inserted into soft matter. For instance, the tube may be inserted into a polymer or a gel. Thus, the device may be expelled from the tube such that the device at least partially penetrates into the target.

As mentioned, in some cases, the device, when inserted into the tube, is constrained or compressed in some fashion such that, upon expulsion (fully or partially), the device is able to at least partially expand. As a non-limiting example, the device may be a network that is rolled to form a cylinder; upon expulsion, the device is able to at least partially unroll and expand. In some cases, the device is able to spontaneously expand, e.g., upon exiting the tube. The expansion may occur rapidly, or on longer time scales. As another example, the device may unfold, or the device may uncompress, upon exiting a tube. The device may expand to reach its original shape. In some cases, the device may substantially return to its original shape after about 24 hours, after about 48 hours, or after about 72 hours. In certain embodiments, it may take longer for the device to substantially return to its original shape, e.g., after 1 week, after 2 weeks, after 3 weeks, after 4 weeks, after 5 weeks, after 6 weeks, etc. In some cases, however, the device may not necessarily return to its original shape, e.g., inherently, and/or due to the matter that the device was injected or inserted into. For example, the presence of tissue (or other matter) may prevent the device from fully expanding back to its original shape after insertion.

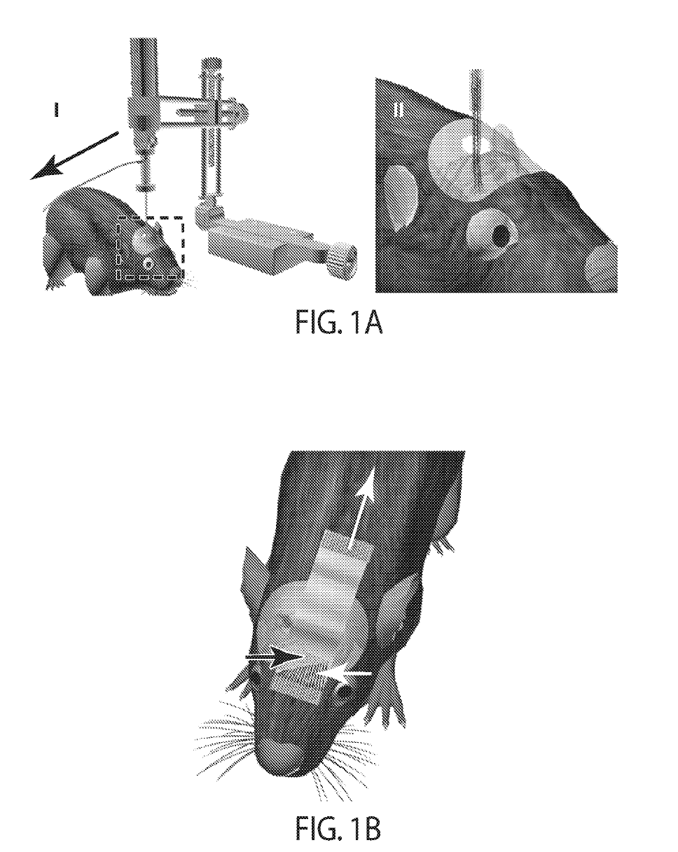

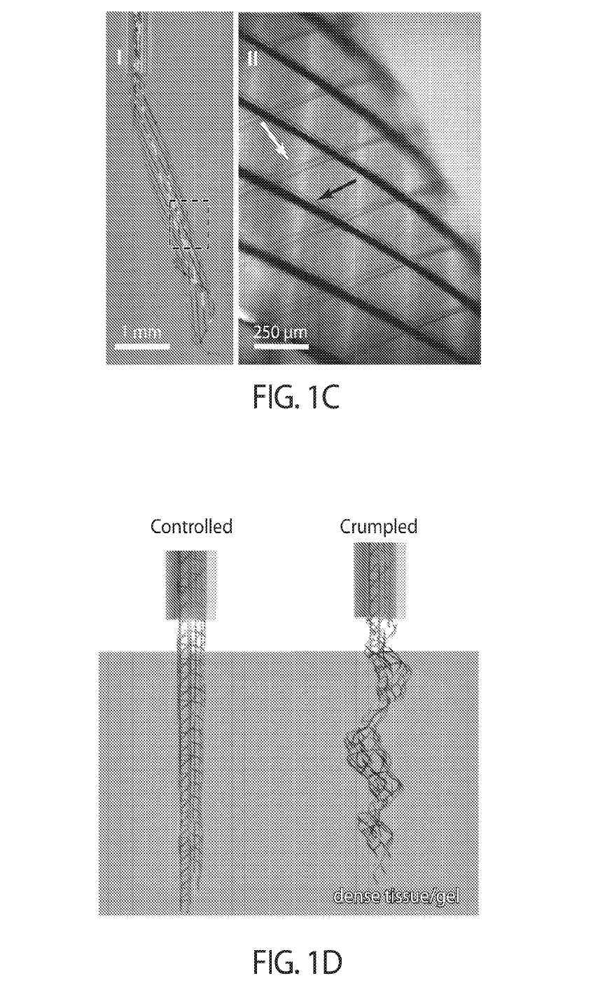

In addition, in some embodiments of the invention, the device may be expelled or urged from a tube (or other suitable carrier) such that the device is not significantly distorted, e.g., due to mechanical resistance offered by the medium that the device is being inserted into. In some embodiments, the device may be expelled or urged from a tube without substantially altering the position of the device relative to the medium. This may be useful, for example, to prevent or minimize compressive forces on the device as it encounters the medium, e.g., which may deform or “crumple” the device. See, e.g., FIG. 1D, showing a “crumpled” device.

In some cases, the device may be “at rest” relative to the medium while the tube is removed. In other embodiments, however, there may be some relative motion, e.g., due to forces involved in removing the tube and/or urging the device out of the tube, movement of the medium (e.g., if the medium is alive), etc. In some cases, the motion may be less than about 10 cm/s, less than about 5 cm/s, less than about 3 cm/s, less than amount 1 cm/s, less than about 5 mm/s, less than about 3 mm/s, less than about 1 mm/s, less than about 0.5 mm/s, less than about 0.3 mm/s, or less than about 0.1 mm/s. Thus, the position of the device, relative to the medium, may not change substantially, or the position may change by no more than about 40%, no more than about 35%, no more than about 30%, no more than about 25%, no more than about 20%, no more than about 15%, no more than about 10%, no more than about 5%, no more than about 2%, or no more than about 1%, relative to the length of the device. In another set of embodiments, the position of the device, relative to the medium, may change by no more than about 1 mm, no more than about 800 micrometers, no more than about 500 micrometers, no more than about 400 micrometers, no more than about 300 micrometers, no more than about 200 micrometers, no more than about 100 micrometers, no more than about 80 micrometers, no more than about 50 micrometers, no more than about 30 micrometers, no more than about 20 micrometers, no more than about 10 micrometers, no more than about 5 micrometers, etc.

This may be accomplished, for example, by withdrawing the tube from the medium while simultaneously urging the device out of the tube, e.g., such that these rates are substantially comparable. In some cases, the rates may differ by no more than about 40%, no more than about 35%, no more than about 30%, no more than about 25%, no more than about 20%, no more than about 15%, no more than about 10%, no more than about 5%, no more than about 2%, or no more than about 1%, relative to the slower of the two rates. In one embodiment, the rates are substantially equal.

As another example, the tube may be removed from the medium by dissolving or liquefying the tube. For example, the tube may be formed from frozen saline, or another suitably benign (or biocompatible) material, and after insertion, the tube is simply allowed to melt while within the medium, thereby leaving the device behind without substantially altering the position of the device, relative to the medium. As another example, the tube may be formed from a biodegradable polymer, such as polylactic acid, polyglycolic acid, polycaprolactone, etc.

In some aspects, other materials may also be present within the tube, e.g., in addition to the device. For example, in one set of embodiments, a gas or a liquid may be present within the tube. For instance, the tube may contain a liquid to facilitate expulsion of the device, or a liquid to assist in movement of the device out of the tube, or into the target. For instance, the tube may include a liquid such as saline, which can be injected into a subject, e.g., along with the device. In addition, in some cases, the fluid may also contain one or more cells, which may be inserted or injected into a target along with the device. If the target is a subject or biological tissue, the cells may be autologous, heterologous, or homologous to the tissue or to the subject.

In certain aspects, the device may comprise one or more electrical networks comprising nanoscale sensing elements and/or nanoscale wires and conductive pathways in electrical communication with the nanoscale sensing elements or nanoscale wires. In some cases, at least some of the conductive pathways may also provide mechanical strength to the device, and/or there may be polymeric or metal constructs that are used to provide mechanical strength to the device. The device may be planar or substantially define a plane, or the device may be non-planar or curved (i.e., a surface that can be characterized as having a finite radius of curvature). The device may also be flexible in some cases, e.g., the device may be able to bend or flex. For example, a device may be bent or distorted by a volumetric displacement of at least about 5%, about 10%, or about 20% (relative to the undisturbed volume), without causing cracks and/or breakage within the device. For example, in some cases, the device can be distorted such that about 5%, about 10%, or about 20% of the mass of the device has been moved outside the original surface perimeter of the device, without causing failure of the device (e.g., by breaking or cracking of the device, disconnection of portions of the electrical network, etc.). In some cases, the device may be bent or flexed as described above by an ordinary human being without the use of tools, machines, mechanical device, excessive force, or the like. A flexible device may be more biocompatible due to its flexibility, and the device may be treated as previously discussed to facilitate its insertion into a tissue.

In addition, the device may be non-planar in some cases, e.g., curved as previously discussed. For example, the device may be substantially U-shaped or cylindrical, and/or have a shape and/or size that is similar to a hypodermic needle. In some embodiments, the device may be generally cylindrical with a maximum outer diameter of no more than about 5 mm, no more than about 4 mm, no more than about 3 mm, no more than about 2 mm, no more than about 1 mm, no more than about 0.9 mm, no more than about 0.8 mm, no more than about 0.7 mm, no more than about 0.6 mm, no more than about 0.5 mm, no more than about 0.4 mm, no more than about 0.3 mm, or no more than about 0.2 mm. Accordingly, in some embodiments, the device may be able to be placed into a tube, e.g., of a needle or a syringe. As discussed herein, the device can then be inserted or injected out of the tube upon application of suitable forces and/or pressures, for instance, such that the device can be inserted or injected into other matter. For instance, the device may be injected into the tissue of a subject, or into a gel.

In one aspect, the device may comprise a periodic structure comprising nanoscale sensing elements and/or other nanoscale wires. For example, the device may comprise a mesh or other two-dimensional array of nanoscale sensing elements and/or other nanoscale wires and conductive pathways. The mesh may include a first set of conductive pathways, generally parallel to each other, and a second set of conductive pathways, generally parallel to each other. The first set and the second set may be orthogonal to each other, or they may cross at any suitable angle. For instance, the sets may cross at a 30° angle, a 45° angle, or a 60° angle, or any other suitable angle. Mesh structures of the device may be particularly useful in certain embodiments. For instance, in a mesh structure, due to the physical connections, it may be easier for the structure to maintain its topological configuration, e.g., of the nanoscale wires and/or nanoscale sensing elements relative to each other. In addition, it may be more difficult for the structure to become adversely tangled. If a periodic structure is used, the period may be of any suitable length. For example, the length of a unit cell within the periodic structure may be less than about less than about 500 micrometers, less than about 400 micrometers, less than about 300 micrometers, less than about 200 micrometers, less than about 100 micrometers, less than about 80 micrometers, less than about 60 micrometers, less than about 50 micrometers, etc.

In certain aspects, the device may contain one or more polymeric constructs. The polymeric constructs typically comprise one or more polymers, e.g., photoresists, biocompatible polymers, biodegradable polymers, etc., and optionally may contain other materials, for example, metal leads or other conductive pathway materials. The polymeric constructs may be separately formed then assembled into the device, and/or the polymeric constructs may be integrally formed as part of the device, for example, by forming or manipulating (e.g. folding, rolling, etc.) the polymeric constructs into a 3-dimensional structure that defines the device.

In one set of embodiments, some or all of the polymeric constructs have the form of fibers or ribbons. For example, the polymeric constructs may have one dimension that is substantially longer than the other dimensions of the polymeric construct. The fibers can in some cases be joined together to form a network or mesh of fibers. For example, a device may contain a plurality of fibers that are orthogonally arranged to form a regular network of polymeric constructs. However, the polymeric constructs need not be regularly arranged. The polymer constructs may have the form of fibers or other shapes. In general, any shape or dimension of polymeric construct may be used to form a device.

In one set of embodiments, some or all of the polymeric constructs have a smallest dimension or a largest cross-sectional dimension of less than about 5 micrometers, less than about 4 micrometers, less than about 3 micrometers, less than about 2 micrometers, less than about 1 micrometer, less than about 700 nm, less than about 600 nm, less than about 500 nm, less than about 300 nm, less than about 200 nm, less than about 100 nm, less than about 80 nm, less than about 50 nm, less than about 30 nm, less than about 10 nm, less than about 5 nm, less than about 2 nm, etc. A polymeric construct may also have any suitable cross-sectional shape, e.g., circular, square, rectangular, polygonal, elliptical, regular, irregular, etc. Examples of methods of forming polymeric constructs, e.g., by lithographic or other techniques, are discussed below.

In one set of embodiment, the polymeric constructs can be arranged such that the device is relatively porous, e.g., such that cells can penetrate into the device after insertion of the device. For example, in some cases, the polymeric constructs may be constructed and arranged within the device such that the device has an open porosity of at least about 30%, at least about 40%, at least about 50%, at least about 60%, at least about 70%, at least about 75%, at least about 80%, at least about 85%, at least about 90%, at least about 95%, at least about 97, at least about 99%, at least about 99.5%, or at least about 99.8%. The “open porosity” is generally described as the volume of empty space within the device divided by the overall volume defined by the device, and can be thought of as being equivalent to void volume. Typically, the open porosity includes the volume within the device to which cells can access. In some cases, the device does not contain significant amounts of internal volume to which the cells are incapable of addressing, e.g., due to lack of access and/or pore access being too small.

In some cases, a “two-dimensional open porosity” may also be defined, e.g., of a device that is subsequently formed or manipulated into a 3-dimensional structure. The two-dimensional open porosities of a device can be defined as the void area within the two-dimensional configuration of the device (e.g., where no material is present) divided by the overall area of device, and can be determined before or after the device has been formed into a 3-dimensional structure. Depending on the application, a device may have a two-dimensional open porosity of at least about 30%, at least about 40%, at least about 50%, at least about 60%, at least about 70%, at least about 75%, at least about 80%, at least about 85%, at least about 90%, at least about 95%, at least about 97, at least about 99%, at least about 99.5%, or at least about 99.8%, etc.

Another method of generally determining the two-dimensional porosity of the device is by determining the areal mass density, i.e., the mass of the device divided by the area of one face of the device (including holes or voids present therein). Thus, for example, in another set of embodiments, the device may have an areal mass density of less than about 100 micrograms/cm², less than about 80 micrograms/cm², less than about 60 micrograms/cm², less than about 50 micrograms/cm², less than about 40 micrograms/cm², less than about 30 micrograms/cm², or less than about 20 micrograms/cm².

The porosity of a device can be defined by one or more pores. Pores that are too small can hinder or restrict cell access. Thus, in one set of embodiments, the device may have an average pore size of at least about 100 micrometers, at least about 200 micrometers, at least about 300 micrometers, at least about 400 micrometers, at least about 500 micrometers, at least about 600 micrometers, at least about 700 micrometers, at least about 800 micrometers, at least about 900 micrometers, or at least about 1 mm. However, in other embodiments, pores that are too big may prevent cells from being able to satisfactorily use or even access the pore volume. Thus, in some cases, the device may have an average pore size of no more than about 1.5 mm, no more than about 1.4 mm, no more than about 1.3 mm, no more than about 1.2 mm, no more than about 1.1 mm, no more than about 1 mm, no more than about 900 micrometers, no more than about 800 micrometers, no more than about 700 micrometers, no more than about 600 micrometers, or no more than about 500 micrometers. Combinations of these are also possible, e.g., in one embodiment, the average pore size is at least about 100 micrometers and no more than about 1.5 mm. In addition, larger or smaller pores than these can also be used in a device in certain cases. Pore sizes may be determined using any suitable technique, e.g., through visual inspection (e.g., of microscope images), BET measurements, or the like.

In various embodiments, one or more of the polymers forming a polymeric construct may be a photoresist. While not commonly used in biological devices, photoresists are typically used in lithographic techniques, which can be used as discussed herein to form the polymeric construct. For example, the photoresist may be chosen for its ability to react to light to become substantially insoluble (or substantially soluble, in some cases) to a photoresist developer. For instance, photoresists that can be used within a polymeric construct include, but are not limited to, SU-8, S1805, LOR 3A, poly(methyl methacrylate), poly(methyl glutarimide), phenol formaldehyde resin (diazonaphthoquinone/novolac), diazonaphthoquinone (DNQ), Hoechst AZ 4620, Hoechst AZ 4562, Shipley 1400-17, Shipley 1400-27, Shipley 1400-37, or the like. These and many other photoresists are available commercially.

A polymeric construct may also contain one or more polymers that are biocompatible and/or biodegradable, in certain embodiments. A polymer can be biocompatible, biodegradable, or both biocompatible and biodegradable, and in some cases, the degree of biodegradation or biocompatibility depends on the physiological environment to which the polymer is exposed to.

Typically, a biocompatible material is one that does not illicit an immune response, or elicits a relatively low immune response, e.g., one that does not impair the device or the cells therein from continuing to function for its intended use. In some embodiments, the biocompatible material is able to perform its desired function without eliciting any undesirable local or systemic effects in the subject. In some cases, the material can be incorporated into tissues within the subject, e.g., without eliciting any undesirable local or systemic effects, or such that any biological response by the subject does not substantially affect the ability of the material from continuing to function for its intended use. For example, in a device, the device may be able to determine cellular or tissue activity after insertion, e.g., without substantially eliciting undesirable effects in those cells, or undesirable local or systemic responses, or without eliciting a response that causes the device to cease functioning for its intended use. Examples of techniques for determining biocompatibility include, but are not limited to, the ISO 10993 series for evaluating the biocompatibility of medical devices. As another example, a biocompatible material may be implanted in a subject for an extended period of time, e.g., at least about a month, at least about 6 months, or at least about a year, and the integrity of the material, or the immune response to the material, may be determined. For example, a suitably biocompatible material may be one in which the immune response is minimal, e.g., one that does not substantially harm the health of the subject. One example of a biocompatible material is poly(methyl methacrylate). In some embodiments, a biocompatible material may be used to cover or shield a non-biocompatible material (or a poorly biocompatible material) from the cells or tissue, for example, by covering the material.

A biodegradable material typically degrades over time when exposed to a biological system, e.g., through oxidation, hydrolysis, enzymatic attack, phagocytosis, or the like. For example, a biodegradable material can degrade over time when exposed to water (e.g., hydrolysis) or enzymes. In some cases, a biodegradable material is one that exhibits degradation (e.g., loss of mass and/or structure) when exposed to physiological conditions for at least about a month, at least about 6 months, or at least about a year. For example, the biodegradable material may exhibit a loss of mass of at least about 10%, at least about 20%, at least about 30%, at least about 40%, at least about 50%, at least about 60%, at least about 70%, at least about 80%, or at least about 90%. In certain cases, some or all of the degradation products may be resorbed or metabolized, e.g., into cells or tissues. For example, certain biodegradable materials, during degradation, release substances that can be metabolized by cells or tissues. For instance, polylactic acid releases lactic acid during degradation.

Examples of such biocompatible and/or biodegradable polymers include, but are not limited to, poly(lactic-co-glycolic acid), polylactic acid, polyglycolic acid, poly(methyl methacrylate), poly(trimethylene carbonate), collagen, fibrin, polysaccharidic materials such as chitosan or glycosaminoglycans, hyaluronic acid, polycaprolactone, and the like.

The polymers and other components forming the device can also be used in some embodiments to provide a certain degree of flexibility to the device, which can be quantified as a bending stiffness per unit width of polymer construct. In various embodiments, the overall device may have a bending stiffness of less than about 5 nN m, less than about 4.5 nN m, less than about 4 nN m, less than about 3.5 nN m, less than about 3 nN m, less than about 2.5 nN m, less than about 2 nN m, less than about 1.5 nN m, less than about 1 nN m, less than about 0.5 nM m, less than about 0.3 nM m, less than about 0.1 nM m, less than about 0.05 nM m, less than about 0.03 nM m, less than about 0.01 nM m, less than about 0.005 nM m, less than about 0.003 nM m, less than about 0.001 nM m, less than about 0.0005 nM m, less than about 0.0003 nM m, etc. In some cases, devices having relatively low bending stiffnesses are relatively flexible and bendable, and can be readily inserted into a tube, as discussed herein.

In some embodiments of the invention, the device may also contain other materials in addition to the photoresists or biocompatible and/or biodegradable polymers described above. Non-limiting examples include other polymers, growth hormones, extracellular matrix protein, specific metabolites or nutrients, or the like. For example, in one of embodiments, one or more agents able to promote cell growth can be added to the device, e.g., hormones such as growth hormones, extracellular matrix protein, pharmaceutical agents, vitamins, or the like. Many such growth hormones are commercially available, and may be readily selected by those of ordinary skill in the art based on the specific type of cell or tissue used or desired. Similarly, non-limiting examples of extracellular matrix proteins include gelatin, laminin, fibronectin, heparan sulfate, proteoglycans, entactin, hyaluronic acid, collagen, elastin, chondroitin sulfate, keratan sulfate, Matrigel™, or the like. Many such extracellular matrix proteins are available commercially, and also can be readily identified by those of ordinary skill in the art based on the specific type of cell or tissue used or desired.

As another example, in one set of embodiments, additional materials can be added to the device, e.g., to control the size of pores within the device, to promote cell adhesion or cell growth within the device, to increase the structural stability of the device, to control the flexibility of the device, etc. For instance, in one set of embodiments, additional fibers or other suitable polymers may be added to the device, e.g., electrospun fibers can be used as a secondary scaffold. The additional materials can be formed from any of the materials described herein, e.g., photoresists or biocompatible and/or biodegradable polymers, or other polymers described herein. As another non-limiting example, a glue such as a silicone elastomer glue or dental cement can be used to control the shape of the device.

In some cases, the device can include a 2-dimensional structure that is formed into a final 3-dimensional structure, e.g., by folding or rolling the structure. It should be understood that although the 2-dimensional structure can be described as having an overall length, width, and height, the overall length and width of the structure may each be substantially greater than the overall height of the structure. The 2-dimensional structure may also be manipulated to have a different shape that is 3-dimensional, e.g., having an overall length, width, and height where the overall length and width of the structure are not each substantially greater than the overall height of the structure. For instance, the structure may be manipulated to increase the overall height of the material, relative to its overall length and/or width, for example, by folding or rolling the structure. Thus, for example, a relatively planar sheet of material (having a length and width much greater than its thickness) may be rolled up into a “tube,” such that the tube has an overall length, width, and height of relatively comparable dimensions).

Thus, for example, the 2-dimensional structure may comprise one or more nanoscale wires and/or nanoscale sensing elements and one or more polymeric constructs formed into a 2-dimensional structure or network that is subsequently formed into a 3-dimensional structure. In some embodiments, the 2-dimesional structure may be rolled or curled up to form the 3-dimesional structure, or the 2-dimensional structure may be folded or creased one or more times to form the 3-dimesional structure. Such manipulations can be regular or irregular. In certain embodiments, as discussed herein, the manipulations are caused by pre-stressing the 2-dimensional structure such that it spontaneously forms the 3-dimensional structure, although in other embodiments, such manipulations can be performed separately, e.g., after formation of the 2-dimensional structure.

In some aspects, the device may include one or more metal leads or electrodes, or other conductive pathways. The metal leads or conductive pathways may provide mechanical support, and/or one or more metal leads can be used within a conductive pathway to a nanoscale sensing element (or other nanoscale wire). The metal lead may directly physically contact the nanoscale sensing element and/or there may be other materials between the metal lead and the nanoscale sensing element that allow electrical communication to occur. In some cases, one or more metal leads or other conductive pathways may extend such that the device can be connected to external electrical circuits, computers, or the like, e.g., as discussed herein. Metal leads are useful due to their high conductance, e.g., such that changes within electrical properties obtained from the conductive pathway can be related to changes in properties of the nanoscale sensing element, rather than changes in properties of the conductive pathway. However, it is not a requirement that only metal leads be used, and in other embodiments, other types of conductive pathways may also be used, in addition or instead of metal leads.

A wide variety of metal leads can be used, in various embodiments of the invention. As non-limiting examples, the metals used within a metal lead may include aluminum, gold, silver, copper, molybdenum, tantalum, titanium, nickel, tungsten, chromium, palladium, platinum, as well as any combinations of these and/or other metals. In some cases, the metal can be chosen to be one that is readily introduced into the device, e.g., using techniques compatible with lithographic techniques. For example, in one set of embodiments, lithographic techniques such as e-beam lithography, photolithography, X-ray lithography, extreme ultraviolet lithography, ion projection lithography, etc. may be used to layer or deposit one or more metals on a substrate. Additional processing steps can also be used to define or register the metal leads in some cases. Thus, for example, the thickness of a metal layer may be less than about 5 micrometers, less than about 4 micrometers, less than about 3 micrometers, less than about 2 micrometers, less than about 1 micrometer, less than about 700 nm, less than about 600 nm, less than about 500 nm, less than about 300 nm, less than about 200 nm, less than about 100 nm, less than about 80 nm, less than about 50 nm, less than about 30 nm, less than about 10 nm, less than about 5 nm, less than about 2 nm, etc. The thickness of the layer may also be at least about 10 nm, at least about 20 nm, at least about 40 nm, at least about 60 nm, at least about 80 nm, or at least about 100 nm. For example, the thickness of a layer may be between about 40 nm and about 100 nm, between about 50 nm and about 80 nm.

In some embodiments, more than one metal can be used within a metal lead. For example, two, three, or more metals may be used within a metal lead. The metals may be deposited in different regions or alloyed together, or in some cases, the metals may be layered on top of each other, e.g., layered on top of each other using various lithographic techniques. For example, a second metal may be deposited on a first metal, and in some cases, a third metal may be deposited on the second metal, etc. Additional layers of metal (e.g., fourth, fifth, sixth, etc.) may also be used in some embodiments. The metals can all be different, or in some cases, some of the metals (e.g., the first and third metals) may be the same. Each layer may independently be of any suitable thickness or dimension, e.g., of the dimensions described above, and the thicknesses of the various layers can independently be the same or different.

If dissimilar metals are layered on top of each other, they may be layered in some embodiments in a “stressed” configuration (although in other embodiments they may not necessarily be stressed). As a specific non-limiting example, chromium and palladium can be layered together to cause stresses in the metal leads to occur, thereby causing warping or bending of the metal leads. The amount and type of stress may also be controlled, e.g., by controlling the thicknesses of the layers. For example, relatively thinner layers can be used to increase the amount of warping that occurs.

Without wishing to be bound by any theory, it is believed that layering metals having a difference in stress (e.g., film stress) with respect to each other may, in some cases, cause stresses within the metal, which can cause bending or warping as the metals seek to relieve the stresses. In some embodiments, such mismatches are undesirable because they could cause warping of the metal leads and thus, the device. However, in other embodiments, such mismatches may be desired, e.g., so that the device can be intentionally deformed to form a 3-dimensional structure, as discussed below. In addition, in certain embodiments, the deposition of mismatched metals within a lead may occur at specific locations within the device, e.g., to cause specific warpings to occur, which can be used to cause the device to be deformed into a particular shape or configuration. For example, a “line” of such mismatches can be used to cause an intentional bending or folding along the line of the device.

The device may include one or more nanoscale sensing elements and/or nanoscale wires, which may be the same or different from each other, in accordance with various aspects of the invention. Non-limiting examples of such nanoscale wires (or other nanoscale sensing element) are discussed in detail below, and include, for instance, semiconductor nanowires, carbon nanotubes, or the like. In some cases, at least one of the nanoscale sensing elements is a silicon nanowire. The nanoscale sensing elements may also be straight, or kinked in some cases. In some embodiments, one or more of the nanoscale sensing elements may form at least a portion of a transistor, such as a field-effect transistor, e.g., as is discussed in more detail below. The nanoscale sensing elements may be distributed within the device in any suitable configuration, for example, in an ordered array or randomly distributed. In some cases, the nanoscale sensing elements are distributed such that an increasing concentration of nanoscale sensing elements can be found towards the portion of the device that is first inserted.

In some cases, some or all of the nanoscale wires are individually electronically addressable within the device. For instance, in some cases, at least about 10%, at least about 20%, at least about 30%, at least about 40%, at least about 50%, at least about 60%, at least about 70%, at least about 80%, at least about 90%, or substantially all of the nanoscale wires may be individually electronically addressable. In some embodiments, an electrical property of a nanoscale wire can be individually determinable (e.g., being partially or fully resolvable without also including the electrical properties of other nanoscale wires), and/or such that the electrical property of a nanoscale wire may be individually controlled (for example, by applying a desired voltage or current to the nanoscale wire, for instance, without simultaneously applying the voltage or current to other nanoscale wires). In other embodiments, however, at least some of the nanoscale wires can be controlled within the same electronic circuit (e.g., by incorporating the nanoscale wires in series and/or in parallel), such that the nanoscale wires can still be electronically controlled and/or determined.

In various embodiments, more than one nanoscale wire (or other nanoscale sensing element) may be present within the device. The nanoscale wires may each independently be the same or different. For example, the device may comprise at least 5 nanoscale wires, at least about 10 nanoscale wires, at least about 15 nanoscale wires, at least about 20 nanoscale wires, at least about 25 nanoscale wires, at least about 30 nanoscale wires, at least about 50 nanoscale wires, at least about 100 nanoscale wires, at least about 300 nanoscale wires, at least about 1000 nanoscale wires, etc.

In addition, in some embodiments, there may be a relatively high density of nanoscale wires (or other nanoscale sensing elements) within the device, or at least a portion of the device. The nanoscale wires may be distributed uniformly or non-uniformly on the device. In some cases, the nanoscale wires may be distributed at an average density of at least about 5 wires/mm², at least about 10 wires/mm², at least about 30 wires/mm², at least about 50 wires/mm², at least about 75 wires/mm², at least about 100 wires/mm², at least about 300 wires/mm², at least about 500 wires/mm², at least about 750 wires/mm², at least about 1000 wires/mm², etc. In certain embodiments, the nanoscale wires are distributed such that the average separation between a nanoscale wire and its nearest neighboring nanoscale wire is less than about 2 mm, less than about 1 mm, less than about 500 micrometers, less than about 300 micrometers, less than about 100 micrometers, less than about 50 micrometers, less than about 30 micrometers, or less than about 10 micrometers.

Some or all of the nanoscale wires (or other nanoscale sensing element) may be in electrical communication with one or more electrical connectors via one or more conductive pathways. The electrical connectors may be positioned on a portion of the device that is not inserted into the tissue. The electrical connectors may be made out of any suitable material that allows transmission of an electrical signal. For example, the electrical connectors may comprise gold, silver, copper, aluminum, tantalum, titanium, nickel, tungsten, chromium, palladium, etc. In some cases, the electrical connectors have an average cross-section of less than about 10 micrometers, less than about 8 micrometers, less than about 6 micrometers, less than about 5 micrometers, less than about 4 micrometers, less than about 3 micrometers, less than about 2 micrometers, less than about 1 micrometer, etc.

In some embodiments, the electrical connectors can be used to determine a property of a nanoscale wire (or other nanoscale sensing element) within the device (for example, an electrical property or a chemical property as is discussed herein), and/or to direct an electrical signal to a nanoscale wire, e.g., to electrically stimulate cells proximate the nanoscale wire. The conductive pathways can form an electrical circuit that is internally contained within the device, and/or that extends externally of the device, e.g., such that the electrical circuit is in electrical communication with an external electrical system, such as a computer or a transmitter (for instance, a radio transmitter, a wireless transmitter, an Internet connection, etc.). Any suitable pathway conductive pathway may be used, for example, pathways comprising metals, semiconductors, conductive polymers, or the like.

Furthermore, more than one conductive pathway may be used in certain embodiments. For example, multiple conductive pathways can be used such that some or all of the nanoscale wires within the device may be electronically individually addressable, as previously discussed. However, in other embodiments, more than one nanoscale wire may be addressable by a particular conductive pathway. In addition, in some cases, other electronic components may also be present within the device, e.g., as part of a conductive pathway or otherwise forming part of an electrical circuit. Examples include, but are not limited to, transistors such as field-effect transistors or bipolar junction transistors, resistors, capacitors, inductors, diodes, integrated circuits, etc. In certain cases, some of these may also comprise nanoscale wires. For example, in some embodiments, two sets of electrical connectors and conductive pathways, and a nanoscale wire, may be used to define a transistor such as a field effect transistor, e.g., where the nanoscale wire defines the gate. As mentioned, the environment in and/or around the nanoscale wire can affect the ability of the nanoscale wire to function as a gate.

As mentioned, in various embodiments, one or more electrodes, electrical connectors, and/or conductive pathways may be positioned in electrical and/or physical communication with the nanoscale wires. These can be patterned to be in direct physical contact the nanoscale wire and/or there may be other materials that allow electrical communication to occur. Metals may be used due to their high conductance, e.g., such that changes within electrical properties obtained from the conductive pathway may be related to changes in properties of the nanoscale wire, rather than changes in properties of the conductive pathway. However, in other embodiments, other types of electrode materials are used, in addition or instead of metals.

A wide variety of metals may be used in various embodiments of the invention, for example in an electrode, electrical connector, conductive pathway, metal construct, polymer construct, etc. As non-limiting examples, the metals may include one or more of aluminum, gold, silver, copper, molybdenum, tantalum, titanium, nickel, tungsten, chromium, palladium, as well as any combinations of these and/or other metals. In some cases, the metal may be chosen to be one that is readily introduced, e.g., using techniques compatible with lithographic techniques. For example, in one set of embodiments, lithographic techniques such as e-beam lithography, photolithography, X-ray lithography, extreme ultraviolet lithography, ion projection lithography, etc. can be used to pattern or deposit one or more metals.

Additional processing steps can also be used to define or register the electrode, electrical connector, conductive pathway, metal construct, polymer construct, etc. in some cases. Thus, for example, the thickness of one of these may be less than about 5 micrometers, less than about 4 micrometers, less than about 3 micrometers, less than about 2 micrometers, less than about 1 micrometer, less than about 700 nm, less than about 600 nm, less than about 500 nm, less than about 300 nm, less than about 200 nm, less than about 100 nm, less than about 80 nm, less than about 50 nm, less than about 30 nm, less than about 10 nm, less than about 5 nm, less than about 2 nm, etc. The thickness of the electrode may also be at least about 10 nm, at least about 20 nm, at least about 40 nm, at least about 60 nm, at least about 80 nm, or at least about 100 nm. For example, the thickness of an electrode may be between about 40 nm and about 100 nm, between about 50 nm and about 80 nm.

In some embodiments, more than one metal may be used. The metals can be deposited in different regions or alloyed together, or in some cases, the metals may be layered on top of each other, e.g., layered on top of each other using various lithographic techniques. For example, a second metal may be deposited on a first metal, and in some cases, a third metal may be deposited on the second metal, etc. Additional layers of metal (e.g., fourth, fifth, sixth, etc.) can also be used in some embodiments. The metals may all be different, or in some cases, some of the metals (e.g., the first and third metals) may be the same. Each layer may independently be of any suitable thickness or dimension, e.g., of the dimensions described above, and the thicknesses of the various layers may independently be the same or different.

As mentioned, any nanoscale wire can be used in the device, e.g., as a nanoscale sensing element. Non-limiting examples of suitable nanoscale wires include carbon nanotubes, nanorods, nanowires, organic and inorganic conductive and semiconducting polymers, metal nanoscale wires, semiconductor nanoscale wires (for example, formed from silicon), and the like. If carbon nanotubes are used, they may be single-walled and/or multi-walled, and may be metallic and/or semiconducting in nature. Other conductive or semiconducting elements that may not be nanoscale wires, but are of various small nanoscopic-scale dimension, also can be used in certain embodiments.

In general, a “nanoscale wire” (also known herein as a “nanoscopic-scale wire” or “nanoscopic wire”) generally is a wire or other nanoscale object, that at any point along its length, has at least one cross-sectional dimension and, in some embodiments, two orthogonal cross-sectional dimensions (e.g., a diameter) of less than 1 micrometer, less than about 500 nm, less than about 200 nm, less than about 150 nm, less than about 100 nm, less than about 70, less than about 50 nm, less than about 20 nm, less than about 10 nm, less than about 5 nm, than about 2 nm, or less than about 1 nm. In some embodiments, the nanoscale wire is generally cylindrical. In other embodiments, however, other shapes are possible; for example, the nanoscale wire can be faceted, i.e., the nanoscale wire may have a polygonal cross-section. The cross-section of a nanoscale wire can be of any arbitrary shape, including, but not limited to, circular, square, rectangular, annular, polygonal, or elliptical, and may be a regular or an irregular shape. The nanoscale wire can also be solid or hollow.

In some cases, the nanoscale wire has one dimension that is substantially longer than the other dimensions of the nanoscale wire. For example, the nanoscale wire may have a longest dimension that is at least about 1 micrometer, at least about 3 micrometers, at least about 5 micrometers, or at least about 10 micrometers or about 20 micrometers in length, and/or the nanoscale wire may have an aspect ratio (longest dimension to shortest orthogonal dimension) of greater than about 2:1, greater than about 3:1, greater than about 4:1, greater than about 5:1, greater than about 10:1, greater than about 25:1, greater than about 50:1, greater than about 75:1, greater than about 100:1, greater than about 150:1, greater than about 250:1, greater than about 500:1, greater than about 750:1, or greater than about 1000:1 or more in some cases.

In some embodiments, a nanoscale wire are substantially uniform, or have a variation in average diameter of the nanoscale wire of less than about 30%, less than about 25%, less than about 20%, less than about 15%, less than about 10%, or less than about 5%. For example, the nanoscale wires may be grown from substantially uniform nanoclusters or particles, e.g., colloid particles. See, e.g., U.S. Pat. No. 7,301,199, issued Nov. 27, 2007, entitled “Nanoscale Wires and Related Devices,” by Lieber, et al., incorporated herein by reference in its entirety. In some cases, the nanoscale wire may be one of a population of nanoscale wires having an average variation in diameter, of the population of nanowires, of less than about 30%, less than about 25%, less than about 20%, less than about 15%, less than about 10%, or less than about 5%.

In some embodiments, a nanoscale wire has a conductivity of or of similar magnitude to any semiconductor or any metal. The nanoscale wire can be formed of suitable materials, e.g., semiconductors, metals, etc., as well as any suitable combinations thereof. In some cases, the nanoscale wire will have the ability to pass electrical charge, for example, being electrically conductive. For example, the nanoscale wire may have a relatively low resistivity, e.g., less than about 10⁻³ Ohm m, less than about 10⁻⁴ Ohm m, less than about 10⁻⁶ Ohm m, or less than about 10⁻⁷ Ohm m. The nanoscale wire can, in some embodiments, have a conductance of at least about 1 microsiemens, at least about 3 microsiemens, at least about 10 microsiemens, at least about 30 microsiemens, or at least about 100 microsiemens.

The nanoscale wire can be solid or hollow, in various embodiments. As used herein, a “nanotube” is a nanoscale wire that is hollow, or that has a hollowed-out core, including those nanotubes known to those of ordinary skill in the art. As another example, a nanotube may be created by creating a core/shell nanowire, then etching away at least a portion of the core to leave behind a hollow shell. Accordingly, in one set of embodiments, the nanoscale wire is a non-carbon nanotube. In contrast, a “nanowire” is a nanoscale wire that is typically solid (i.e., not hollow). Thus, in one set of embodiments, the nanoscale wire may be a semiconductor nanowire, such as a silicon nanowire.

In one set of embodiment, a nanoscale wire may comprise or consist essentially of a metal. Non-limiting examples of potentially suitable metals include aluminum, gold, silver, copper, molybdenum, tantalum, titanium, nickel, tungsten, chromium, or palladium. In another set of embodiments, a nanoscale wire comprises or consists essentially of a semiconductor. Typically, a semiconductor is an element having semiconductive or semi-metallic properties (i.e., between metallic and non-metallic properties). An example of a semiconductor is silicon. Other non-limiting examples include elemental semiconductors, such as gallium, germanium, diamond (carbon), tin, selenium, tellurium, boron, or phosphorous. In other embodiments, more than one element may be present in the nanoscale wire as the semiconductor, for example, gallium arsenide, gallium nitride, indium phosphide, cadmium selenide, etc. Still other examples include a Group II-VI material (which includes at least one member from Group II of the Periodic Table and at least one member from Group VI, for example, ZnS, ZnSe, ZnSSe, ZnCdS, CdS, or CdSe), or a Group III-V material (which includes at least one member from Group III and at least one member from Group V, for example GaAs, GaP, GaAsP, InAs, InP, AlGaAs, or InAsP). In some cases, at least one of the nanoscale wires is a silicon nanowire.

In certain embodiments, the semiconductor can be undoped or doped (e.g., p-type or n-type). For example, in one set of embodiments, a nanoscale wire may be a p-type semiconductor nanoscale wire or an n-type semiconductor nanoscale wire, and can be used as a component of a transistor such as a field effect transistor (“FET”). For instance, the nanoscale wire may act as the “gate” of a source-gate-drain arrangement of a FET, while metal leads or other conductive pathways (as discussed herein) are used as the source and drain electrodes.

In some embodiments, a dopant or a semiconductor may include mixtures of Group IV elements, for example, a mixture of silicon and carbon, or a mixture of silicon and germanium. In other embodiments, the dopant or the semiconductor may include a mixture of a Group III and a Group V element, for example, BN, BP, BAs, AlN, AlP, AlAs, AlSb, GaN, GaP, GaAs, GaSb, InN, InP, InAs, or InSb. Mixtures of these may also be used, for example, a mixture of BN/BP/BAs, or BN/AlP. In other embodiments, the dopants may include alloys of Group III and Group V elements. For example, the alloys may include a mixture of AlGaN, GaPAs, InPAs, GaInN, AlGaInN, GaInAsP, or the like. In other embodiments, the dopants may also include a mixture of Group II and Group VI semiconductors. For example, the semiconductor may include ZnO, ZnS, ZnSe, ZnTe, CdS, CdSe, CdTe, HgS, HgSe, HgTe, BeS, BeSe, BeTe, MgS, MgSe, or the like. Alloys or mixtures of these dopants are also be possible, for example, (ZnCd)Se, or Zn(SSe), or the like. Additionally, alloys of different groups of semiconductors may also be possible, for example, a combination of a Group II-Group VI and a Group III-Group V semiconductor, for example, (GaAs)ₓ(ZnS)₁₋ₓ. Other examples of dopants may include combinations of Group IV and Group VI elemnts, such as GeS, GeSe, GeTe, SnS, SnSe, SnTe, PbO, PbS, PbSe, or PbTe. Other semiconductor mixtures may include a combination of a Group I and a Group VII, such as CuF, CuCl, CuBr, CuI, AgF, AgCl, AgBr, AgI, or the like. Other dopant compounds may include different mixtures of these elements, such as BeSiN₂, CaCN₂, ZnGeP₂, CdSnAs₂, ZnSnSb₂, CuGeP₃, CuSi₂P₃, Si₃N₄, Ge₃N₄, Al₂O₃, (Al, Ga, In)₂(S, Se, Te)₃, Al₂CO, (Cu, Ag)(Al, Ga, In, Tl, Fe)(S, Se, Te)₂ and the like.

The doping of the semiconductor to produce a p-type or n-type semiconductor may be achieved via bulk-doping in certain embodiments, although in other embodiments, other doping techniques (such as ion implantation) can be used. Many such doping techniques that can be used will be familiar to those of ordinary skill in the art, including both bulk doping and surface doping techniques. A bulk-doped article (e.g. an article, or a section or region of an article) is an article for which a dopant is incorporated substantially throughout the crystalline lattice of the article, as opposed to an article in which a dopant is only incorporated in particular regions of the crystal lattice at the atomic scale, for example, only on the surface or exterior. For example, some articles are typically doped after the base material is grown, and thus the dopant only extends a finite distance from the surface or exterior into the interior of the crystalline lattice. It should be understood that “bulk-doped” does not define or reflect a concentration or amount of doping in a semiconductor, nor does it necessarily indicate that the doping is uniform. “Heavily doped” and “lightly doped” are terms the meanings of which are clearly understood by those of ordinary skill in the art. In some embodiments, one or more regions comprise a single monolayer of atoms (“delta-doping”). In certain cases, the region may be less than a single monolayer thick (for example, if some of the atoms within the monolayer are absent). As a specific example, the regions may be arranged in a layered structure within the nanoscale wire, and one or more of the regions can be delta-doped or partially delta-doped.

Accordingly, in one set of embodiments, the nanoscale wires may include a heterojunction, e.g., of two regions with dissimilar materials or elements, and/or the same materials or elements but at different ratios or concentrations. The regions of the nanoscale wire may be distinct from each other with minimal cross-contamination, or the composition of the nanoscale wire can vary gradually from one region to the next. The regions may be both longitudinally arranged relative to each other, or radially arranged (e.g., as in a core/shell arrangement) on the nanoscale wire. Each region may be of any size or shape within the wire. The junctions may be, for example, a p/n junction, a p/p junction, an n/n junction, a p/i junction (where i refers to an intrinsic semiconductor), an n/i junction, an i/i junction, or the like. The junction can also be a Schottky junction in some embodiments. The junction may also be, for example, a semiconductor/semiconductor junction, a semiconductor/metal junction, a semiconductor/insulator junction, a metal/metal junction, a metal/insulator junction, an insulator/insulator junction, or the like. The junction may also be a junction of two materials, a doped semiconductor to a doped or an undoped semiconductor, or a junction between regions having different dopant concentrations. The junction can also be a defected region to a perfect single crystal, an amorphous region to a crystal, a crystal to another crystal, an amorphous region to another amorphous region, a defected region to another defected region, an amorphous region to a defected region, or the like. More than two regions may be present, and these regions may have unique compositions or may comprise the same compositions. As one example, a wire can have a first region having a first composition, a second region having a second composition, and a third region having a third composition or the same composition as the first composition. Non-limiting examples of nanoscale wires comprising heterojunctions (including core/shell heterojunctions, longitudinal heterojunctions, etc., as well as combinations thereof) are discussed in U.S. Pat. No. 7,301,199, issued Nov. 27, 2007, entitled “Nanoscale Wires and Related Devices,” by Lieber, et al., incorporated herein by reference in its entirety.

In some embodiments, the nanoscale wire is a bent or a kinked nanoscale wire. A kink is typically a relatively sharp transition or turning between a first substantially straight portion of a wire and a second substantially straight portion of a wire. For example, a nanoscale wire may have 1, 2, 3, 4, or 5 or more kinks. In some cases, the nanoscale wire is formed from a single crystal and/or comprises or consists essentially of a single crystallographic orientation, for example, a <110> crystallographic orientation, a <112> crystallographic orientation, or a <1120> crystallographic orientation. It should be noted that the kinked region need not have the same crystallographic orientation as the rest of the semiconductor nanoscale wire. In some embodiments, a kink in the semiconductor nanoscale wire may be at an angle of about 120 o or a multiple thereof. The kinks can be intentionally positioned along the nanoscale wire in some cases. For example, a nanoscale wire may be grown from a catalyst particle by exposing the catalyst particle to various gaseous reactants to cause the formation of one or more kinks within the nanoscale wire. Non-limiting examples of kinked nanoscale wires, and suitable techniques for making such wires, are disclosed in International Patent Application No. PCT/US2010/050199, filed Sep. 24, 2010, entitled “Bent Nanowires and Related Probing of Species,” by Tian, et al., published as WO 2011/038228 on Mar. 31, 2011, incorporated herein by reference in its entirety.

In one set of embodiments, the nanoscale wire is formed from a single crystal, for example, a single crystal nanoscale wire comprising a semiconductor. A single crystal item may be formed via covalent bonding, ionic bonding, or the like, and/or combinations thereof. While such a single crystal item may include defects in the crystal in some cases, the single crystal item is distinguished from an item that includes one or more crystals, not ionically or covalently bonded, but merely in close proximity to one another.

In some embodiments, the nanoscale wires used herein are individual or free-standing nanoscale wires. For example, an “individual” or a “free-standing” nanoscale wire may, at some point in its life, not be attached to another article, for example, with another nanoscale wire, or the free-standing nanoscale wire may be in solution. This is in contrast to nanoscale features etched onto the surface of a substrate, e.g., a silicon wafer, in which the nanoscale features are never removed from the surface of the substrate as a free-standing article. This is also in contrast to conductive portions of articles which differ from surrounding material only by having been altered chemically or physically, in situ, i.e., where a portion of a uniform article is made different from its surroundings by selective doping, etching, etc. An “individual” or a “free-standing” nanoscale wire is one that can be (but need not be) removed from the location where it is made, as an individual article, and transported to a different location and combined with different components to make a functional device such as those described herein and those that would be contemplated by those of ordinary skill in the art upon reading this disclosure.

The nanoscale wire, in some embodiments, may be a nanoscale sensing element responsive to a property external of the nanoscale wire, e.g., a chemical property, an electrical property, a physical property, etc. Such determination may be qualitative and/or quantitative, and such determinations may also be recorded, e.g., for later use. For example, in one set of embodiments, the nanoscale wire may be responsive to voltage. For instance, the nanoscale wire may exhibits a voltage sensitivity of at least about 5 microsiemens/V; by determining the conductivity of a nanoscale wire, the voltage surrounding the nanoscale wire may thus be determined. In other embodiments, the voltage sensitivity can be at least about 10 microsiemens/V, at least about 30 microsiemens/V, at least about 50 microsiemens/V, or at least about 100 microsiemens/V. Other examples of electrical properties that can be determined include resistance, resistivity, conductance, conductivity, impendence, or the like.

As another example, a nanoscale wire may be a nanoscale sensing element responsive to a chemical property of the environment surrounding the nanoscale wire. For example, an electrical property of the nanoscale wire can be affected by a chemical environment surrounding the nanoscale wire, and the electrical property can be thereby determined to determine the chemical environment surrounding the nanoscale wire. As a specific non-limiting example, the nanoscale wires may be sensitive to pH or hydrogen ions. Further non-limiting examples of such nanoscale wires are discussed in U.S. Pat. No. 7,129,554, filed Oct. 31, 2006, entitled “Nanosensors,” by Lieber, et al., incorporated herein by reference in its entirety.

As a non-limiting example, the nanoscale wire may be a nanoscale sensing element having the ability to bind to an analyte indicative of a chemical property of the environment surrounding the nanoscale wire (e.g., hydrogen ions for pH, or concentration for an analyte of interest), and/or the nanoscale wire may be partially or fully functionalized, i.e. comprising surface functional moieties, to which an analyte is able to bind, thereby causing a determinable property change to the nanoscale wire, e.g., a change to the resistivity or impedance of the nanoscale wire. The binding of the analyte can be specific or non-specific. Functional moieties may include simple groups, selected from the groups including, but not limited to, —OH, —CHO, —COOH, —SO₃H, 13 CN, —NH₂, —SH, —COSH, —COOR, halide; biomolecular entities including, but not limited to, amino acids, proteins, sugars, DNA, antibodies, antigens, and enzymes; grafted polymer chains with chain length less than the diameter of the nanowire core, selected from a group of polymers including, but not limited to, polyamide, polyester, polyimide, polyacrylic; a shell of material comprising, for example, metals, semiconductors, and insulators, which may be a metallic element, an oxide, an sulfide, a nitride, a selenide, a polymer and a polymer gel. A non-limiting example of a protein is PSA (prostate specific antigen), which can be determined, for example, by modifying the nanoscale wires by binding monoclonal antibodies for PSA (Abl) thereto. See, e.g., U.S. Pat. No. 8,232,584, issued Jul. 31, 2012, entitled “Nanoscale Sensors,” by Lieber, et al., incorporated herein by reference in its entirety.

In some embodiments, a reaction entity may be bound to a surface of the nanoscale wire, and/or positioned in relation to the nanoscale wire such that the analyte can be determined by determining a change in a property of the nanoscale wire, e.g., acting as a nanoscale sensing element. The “determination” may be quantitative and/or qualitative, depending on the application, and in some cases, the determination may also be analyzed, recorded for later use, transmitted, or the like. The term “reaction entity” refers to any entity that can interact with an analyte in such a manner to cause a detectable change in a property (such as an electrical property) of a nanoscale wire. The reaction entity may enhance the interaction between the nanowire and the analyte, or generate a new chemical species that has a higher affinity to the nanowire, or to enrich the analyte around the nanowire. The reaction entity can comprise a binding partner to which the analyte binds. The reaction entity, when a binding partner, can comprise a specific binding partner of the analyte. For example, the reaction entity may be a nucleic acid, an antibody, a sugar, a carbohydrate or a protein. Alternatively, the reaction entity may be a polymer, catalyst, or a quantum dot. A reaction entity that is a catalyst can catalyze a reaction involving the analyte, resulting in a product that causes a detectable change in the nanowire, e.g. via binding to an auxiliary binding partner of the product electrically coupled to the nanowire. Another exemplary reaction entity is a reactant that reacts with the analyte, producing a product that can cause a detectable change in the nanowire. The reaction entity can comprise a shell on the nanowire, e.g. a shell of a polymer that recognizes molecules in, e.g., a gaseous sample, causing a change in conductivity of the polymer which, in turn, causes a detectable change in the nanowire.

The term “binding partner” refers to a molecule that can undergo binding with a particular analyte, or “binding partner” thereof, and includes specific, semi-specific, and non-specific binding partners as known to those of ordinary skill in the art. The term “specifically binds,” when referring to a binding partner (e.g., protein, nucleic acid, antibody, etc.), refers to a reaction that is determinative of the presence and/or identity of one or other member of the binding pair in a mixture of heterogeneous molecules (e.g., proteins and other biologics). Thus, for example, in the case of a receptor/ligand binding pair the ligand would specifically and/or preferentially select its receptor from a complex mixture of molecules, or vice versa. An enzyme would specifically bind to its substrate, a nucleic acid would specifically bind to its complement, an antibody would specifically bind to its antigen. Other examples include, nucleic acids that specifically bind (hybridize) to their complement, antibodies specifically bind to their antigen, and the like. The binding may be by one or more of a variety of mechanisms including, but not limited to ionic interactions, and/or covalent interactions, and/or hydrophobic interactions, and/or van der Waals interactions, etc.

The antibody may be any protein or glycoprotein comprising or consisting essentially of one or more polypeptides substantially encoded by immunoglobulin genes or fragments of immunoglobulin genes. Examples of recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively.

Antibodies exist as intact immunoglobulins or as a number of well characterized fragments produced by digestion with various peptidases. Thus, for example, pepsin digests an antibody below (i.e. toward the Fc domain) the disulfide linkages in the hinge region to produce F(ab)′₂, a dimer of Fab which itself is a light chain joined to VHCH1 by a disulfide bond. The F(ab)′₂ may be reduced under mild conditions to break the disulfide linkage in the hinge region thereby converting the (Fab)₂ dimer into an Fab′ monomer. The Fab′ monomer is essentially a Fab with part of the hinge region. While various antibody fragments are defined in terms of the digestion of an intact antibody, one of skill will appreciate that such fragments may be synthesized de novo either chemically, by utilizing recombinant DNA methodology, or by “phage display” methods. Non-limiting examples of antibodies include single chain antibodies, e.g., single chain Fv (scFv) antibodies in which a variable heavy and a variable light chain are joined together (directly or through a peptide linker) to form a continuous polypeptide.

Thus, in some embodiments, a property such as a chemical property and/or an electrical property can be determined, e.g., at a resolution of less than about 2 mm, less than about 1 mm, less than about 500 micrometers, less than about 300 micrometers, less than about 100 micrometers, less than about 50 micrometers, less than about 30 micrometers, or less than about 10 micrometers, etc., e.g., due to the average separation between a nanoscale wire and its nearest neighboring nanoscale wire. In addition, the property may be determined within the tissue in 3 dimensions in some instances, in contrast with many other techniques where only a surface of the biological tissue can be studied. Accordingly, very high resolution and/or 3-dimensional mappings of the property of the biological tissue can be obtained in some embodiments. Any suitable tissue may be studied, e.g., brain tissue, eyes (e.g., the retina), the spinal cord or other nerves, cardiac tissue, vascular tissue, muscle, cartilage, bone, liver tissue, pancreatic tissue, bladder tissue, airway tissues, bone marrow tissue, or the like.

In addition, in some cases, such properties can be determined and/or recorded as a function of time. Thus, for example, such properties can be determined at a time resolution of less than about 1 min, less than about 30 s, less than about 15 s, less than about 10 s, less than about 5 s, less than about 3 s, less than about 1 s, less than about 500 ms, less than about 300 ms, less than about 100 ms, less than about 50 ms, less than about 30 ms, less than about 10 ms, less than about 5 ms, less than about 3 ms, less than about 1 ms, etc.

In yet another set of embodiments, the biological tissue, and/or portions of the biological tissue, may be electrically stimulated using nanoscale wires present within the tissue. For example, all, or a subset of the electrically active nanoscale wires may be electrically stimulated, e.g., by using an external electrical system, such as a computer. Thus, for example, a single nanoscale wire, a group of nanoscale wires, or substantially all of the nanoscale wires can be electrically stimulated, depending on the particular application. In some cases, such nanoscale wires can be stimulated in a particular pattern, e.g., to cause cardiac or muscle cells to contract or beat in a particular pattern (for example, as part of a prosthetic or a pacemaker), to cause the firing of neurons with a particular pattern, to monitor the status of an implanted tissue within a subject, or the like.

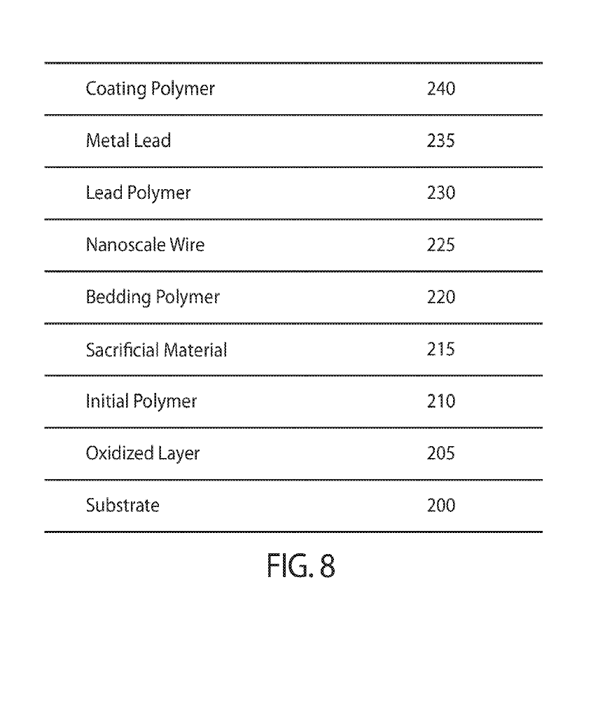

Another aspect of the present invention is generally directed to systems and methods for making and using such devices, e.g., for insertion into matter. Briefly, in one set of embodiments, a device can be constructed by assembling various polymers, metals, nanoscale wires, and other components together on a substrate. For example, lithographic techniques such as e-beam lithography, photolithography, X-ray lithography, extreme ultraviolet lithography, ion projection lithography, etc. may be used to pattern polymers, metals, etc. on the substrate, and nanoscale wires can be prepared separately then added to the substrate. After assembly, at least a portion of the substrate (e.g., a sacrificial material) may be removed, allowing the device to be partially or completely removed from the substrate. The device can, in some cases, be formed into a 3-dimensional structure, for example, spontaneously, or by folding or rolling the structure. Other materials may also be added to the device, e.g., to help stabilize the structure, to add additional agents to enhance its biocompatibility, etc. The device can be used in vivo, e.g., by implanting it in a subject, and/or in vitro, e.g., by seeding cells, etc. on the device. In addition, in some cases, cells may initially be grown on the device before the device is implanted into a subject. A schematic diagram of the layers formed on the substrate in one embodiment is shown in FIG. 8. However, it should be understood that this diagram is illustrative only and is not drawn to scale, and not all of the layers shown in FIG. 8 are necessarily required in every embodiment of the invention.

The substrate (200 in FIG. 8) may be chosen to be one that can be used for lithographic techniques such as e-beam lithography or photolithography, or other lithographic techniques including those discussed herein. For example, the substrate may comprise or consist essentially of a semiconductor material such as silicon, although other substrate materials (e.g., a metal) can also be used. Typically, the substrate is one that is substantially planar, e.g., so that polymers, metals, and the like can be patterned on the substrate.

In some cases, a portion of the substrate can be oxidized, e.g., forming SiO₂ and/or Si₃N₄ on a portion of the substrate, which may facilitate subsequent addition of materials (metals, polymers, etc.) to the substrate. In some cases, the oxidized portion may form a layer of material on the substrate (205 in FIG. 8), e.g., having a thickness of less than about 5 micrometers, less than about 4 micrometers, less than about 3 micrometers, less than about 2 micrometers, less than about 1 micrometer, less than about 900 nm, less than about 800 nm, less than about 700 nm, less than about 600 nm, less than about 500 nm, less than about 400 nm, less than about 300 nm, less than about 200 nm, less than about 100 nm, etc.

In certain embodiments, one or more polymers can also be deposited or otherwise formed prior to depositing the sacrificial material. In some cases, the polymers may be deposited or otherwise formed as a layer of material (210 in FIG. 8) on the substrate. Deposition may be performed using any suitable technique, e.g., using lithographic techniques such as e-beam lithography, photolithography, X-ray lithography, extreme ultraviolet lithography, ion projection lithography, etc. In some cases, some or all of the polymers may be biocompatible and/or biodegradable. The polymers that are deposited may also comprise methyl methacrylate and/or poly(methyl methacrylate), in some embodiments. One, two, or more layers of polymer can be deposited (e.g., sequentially) in various embodiments, and each layer may independently have a thickness of less than about 5 micrometers, less than about 4 micrometers, less than about 3 micrometers, less than about 2 micrometers, less than about 1 micrometer, less than about 900 nm, less than about 800 nm, less than about 700 nm, less than about 600 nm, less than about 500 nm, less than about 400 nm, less than about 300 nm, less than about 200 nm, less than about 100 nm, etc.

Next, a sacrificial material may be deposited. The sacrificial material can be chosen to be one that can be removed without substantially altering other materials (e.g., polymers, other metals, nanoscale wires, etc.) deposited thereon. For example, in one embodiment, the sacrificial material may be a metal, e.g., one that is easily etchable. For instance, the sacrificial material can comprise germanium or nickel, which can be etched or otherwise removed, for example, using a peroxide (e.g., H₂O₂) or a nickel etchant (many of which are readily available commercially). In some cases, the sacrificial material may be deposited on oxidized portions or polymers previously deposited on the substrate. In some cases, the sacrificial material is deposited as a layer (e.g., 215 in FIG. 8). The layer can have a thickness of less than about 5 micrometers, less than about 4 micrometers, less than about 3 micrometers, less than about 2 micrometers, less than about 1 micrometer, less than about 900 nm, less than about 800 nm, less than about 700 nm, less than about 600 nm, less than about 500 nm, less than about 400 nm, less than about 300 nm, less than about 200 nm, less than about 100 nm, etc.

In some embodiments, a “bedding” polymer can be deposited, e.g., on the sacrificial material. The bedding polymer may include one or more polymers, which may be deposited as one or more layers (220 in FIG. 8). The bedding polymer can be used to support the nanoscale wires, and in some cases, partially or completely surround the nanoscale wires, depending on the application. For example, as discussed below, one or more nanoscale wires may be deposited on at least a portion of the uppermost layer of bedding polymer.

For instance, the bedding polymer can at least partially define a device. In one set of embodiments, the bedding polymer may be deposited as a layer of material, such that portions of the bedding polymer may be subsequently removed. For example, the bedding polymer can be deposited using lithographic techniques such as e-beam lithography, photolithography, X-ray lithography, extreme ultraviolet lithography, ion projection lithography, etc., or using other techniques for removing polymer that are known to those of ordinary skill in the art. In some cases, more than one bedding polymer is used, e.g., deposited as more than one layer (e.g., sequentially), and each layer may independently have a thickness of less than about 5 micrometers, less than about 4 micrometers, less than about 3 micrometers, less than about 2 micrometers, less than about 1 micrometer, less than about 900 nm, less than about 800 nm, less than about 700 nm, less than about 600 nm, less than about 500 nm, less than about 400 nm, less than about 300 nm, less than about 200 nm, less than about 100 nm, etc. For example, in some embodiments, portions of the photoresist may be exposed to light (visible, UV, etc.), electrons, ions, X-rays, etc. (e.g., projected onto the photoresist), and the exposed portions can be etched away (e.g., using suitable etchants, plasma, etc.) to produce the pattern.

Accordingly, the bedding polymer may be formed into a particular pattern, e.g., in a grid, or in a pattern that suggests an endogenous probe, before or after deposition of nanoscale wires (as discussed in detail below), in certain embodiments of the invention. The pattern can be regular or irregular. For example, the bedding polymer can be formed into a pattern defining pore sizes such as those discussed herein. For instance, the polymer may have an average pore size of at least about 100 micrometers, at least about 200 micrometers, at least about 300 micrometers, at least about 400 micrometers, at least about 500 micrometers, at least about 600 micrometers, at least about 700 micrometers, at least about 800 micrometers, at least about 900 micrometers, or at least about 1 mm, and/or an average pore size of no more than about 1.5 mm, no more than about 1.4 mm, no more than about 1.3 mm, no more than about 1.2 mm, no more than about 1.1 mm, no more than about 1 mm, no more than about 900 micrometers, no more than about 800 micrometers, no more than about 700 micrometers, no more than about 600 micrometers, or no more than about 500 micrometers, etc.

Any suitable polymer may be used as the bedding polymer. In some cases, one or more of the polymers can be chosen to be biocompatible and/or biodegradable. In certain embodiments, one or more of the bedding polymers may comprise a photoresist. Photoresists can be useful due to their familiarity in use in lithographic techniques such as those discussed herein. Non-limiting examples of photoresists include SU-8, S1805, LOR 3A, poly(methyl methacrylate), poly(methyl glutarimide), phenol formaldehyde resin (diazonaphthoquinone/novolac), diazonaphthoquinone (DNQ), Hoechst AZ 4620, Hoechst AZ 4562, Shipley 1400-17, Shipley 1400-27, Shipley 1400-37, etc., as well as any others discussed herein.

In certain embodiments, one or more of the bedding polymers can be heated or baked, e.g., before or after depositing nanoscale wires thereon as discussed below, and/or before or after patterning the bedding polymer. For example, such heating or baking, in some cases, is important to prepare the polymer for lithographic patterning. In various embodiments, the bedding polymer may be heated to a temperature of at least about 30° C., at least about 65° C., at least about 95° C., at least about 150° C., or at least about 180° C., etc.

Next, one or more nanoscale wires (e.g., 225 in FIG. 8) may be deposited, e.g., on a bedding polymer on the substrate. Any of the nanoscale wires described herein may be used, e.g., n-type and/or p-type nanoscale wires, substantially uniform nanoscale wires (e.g., having a variation in average diameter of less than 20%), nanoscale wires having a diameter of less than about 1 micrometer, semiconductor nanowires, silicon nanowires, bent nanoscale wires, kinked nanoscale wires, core/shell nanowires, nanoscale wires with heterojunctions, etc. In some cases, the nanoscale wires are present in a liquid which is applied to the substrate, e.g., poured, painted, or otherwise deposited thereon. In some embodiments, the liquid is chosen to be relatively volatile, such that some or all of the liquid can be removed by allowing it to substantially evaporate, thereby depositing the nanoscale wires. In some cases, at least a portion of the liquid can be dried off, e.g., by applying heat to the liquid. Examples of suitable liquids include water or isopropanol.

In some cases, at least some of the nanoscale wires may be at least partially aligned, e.g., as part of the deposition process, and/or after the nanoscale wires have been deposited on the substrate. Thus, the alignment can occur before or after drying or other removal of the liquid, if a liquid is used. Any suitable technique may be used for alignment of the nanoscale wires. For example, the nanoscale wires can be aligned by passing or sliding substrates containing the nanoscale wires past each other (see, e.g., International Patent Application No. PCT/US2007/008540, filed Apr. 6, 2007, entitled “Nanoscale Wire Methods and Devices,” by Nam, et al., published as WO 2007/145701 on Dec. 21, 2007, incorporated herein by reference in its entirety), the nanoscale wires can be aligned using Langmuir-Blodgett techniques (see, e.g., U.S. patent application Ser. No. 10/995,075, filed Nov. 22, 2004, entitled “Nanoscale Arrays and Related Devices,” by Whang, et al., published as U.S. Patent Application Publication No. 2005/0253137 on Nov. 17, 2005, incorporated herein by reference in its entirety), the nanoscale wires can be aligned by incorporating the nanoscale wires in a liquid film or “bubble” which is deposited on the substrate (see, e.g., U.S. patent application Ser. No. 12/311,667, filed Apr. 8, 2009, entitled “Liquid Films Containing Nanostructured Materials,” by Lieber, et al., published as U.S. Patent Application Publication No. 2010/0143582 on Jun. 10, 2010, incorporated by reference herein in its entirety), or a gas or liquid can be passed across the nanoscale wires to align the nanoscale wires (see, e.g., U.S. Pat. No. 7,211,464, issued May 1, 2007, entitled “Doped Elongated Semiconductors, Growing Such Semiconductors, Devices Including Such Semiconductors, and Fabricating Such Devices,” by Lieber, et al.; and U.S. Pat. No. 7,301,199, issued Nov. 27, 2007, entitled “Nanoscale Wires and Related Devices,” by Lieber, et al., each incorporated herein by reference in its entirety). Combinations of these and/or other techniques can also be used in certain instances. In some cases, the gas may comprise an inert gas and/or a noble gas, such as nitrogen or argon.

In certain embodiments, a “lead” polymer is deposited (230 in FIG. 8), e.g., on the sacrificial material and/or on at least some of the nanoscale wires. The lead polymer may include one or more polymers, which may be deposited as one or more layers. The lead polymer can be used to cover or protect metal leads or other conductive pathways, which may be subsequently deposited on the lead polymer. In some embodiments, the lead polymer can be deposited, e.g., as a layer of material such that portions of the lead polymer can be subsequently removed, for instance, using lithographic techniques such as e-beam lithography, photolithography, X-ray lithography, extreme ultraviolet lithography, ion projection lithography, etc., or using other techniques for removing polymer that are known to those of ordinary skill in the art, similar to the bedding polymers previously discussed. However, the lead polymers need not be the same as the bedding polymers (although they can be), and they need not be deposited using the same techniques (although they can be). In some cases, more than one lead polymer may be used, e.g., deposited as more than one layer (for example, sequentially), and each layer may independently have a thickness of less than about 5 micrometers, less than about 4 micrometers, less than about 3 micrometers, less than about 2 micrometers, less than about 1 micrometer, less than about 900 nm, less than about 800 nm, less than about 700 nm, less than about 600 nm, less than about 500 nm, less than about 400 nm, less than about 300 nm, less than about 200 nm, less than about 100 nm, etc.

Any suitable polymer can be used as the lead polymer. In some cases, one or more of the polymers may be chosen to be biocompatible and/or biodegradable. For example, in one set of embodiments, one or more of the polymers may comprise poly(methyl methacrylate). In certain embodiments, one or more of the lead polymers comprises a photoresist, such as those described herein.

In certain embodiments, one or more of the lead polymers may be heated or baked, e.g., before or after depositing nanoscale wires thereon as discussed below, and/or before or after patterning the lead polymer. For example, such heating or baking, in some cases, is important to prepare the polymer for lithographic patterning. In various embodiments, the lead polymer may be heated to a temperature of at least about 30° C., at least about 65° C., at least about 95° C., at least about 150° C., or at least about 180° C., etc.

Next, a metal or other conductive material can be deposited (235 in FIG. 8), e.g., on one or more of the lead polymer, the sacrificial material, the nanoscale wires, etc. to form a metal lead or other conductive pathway. More than one metal can be used, which may be deposited as one or more layers. For example, a first metal may be deposited, e.g., on one or more of the lead polymers, and a second metal may be deposited on at least a portion of the first metal. Optionally, more metals can be used, e.g., a third metal may be deposited on at least a portion of the second metal, and the third metal may be the same or different from the first metal. In some cases, each metal may independently have a thickness of less than about 5 micrometers, less than about 4 micrometers, less than about 3 micrometers, less than about 2 micrometers, less than about 1 micrometer, less than about 900 nm, less than about 800 nm, less than about 700 nm, less than about 600 nm, less than about 500 nm, less than about 400 nm, less than about 300 nm, less than about 200 nm, less than about 100 nm, less than about 80 nm, less than about 60 nm, less than about 40 nm, less than about 30 nm, less than about 20 nm, less than about 10 nm, less than about 8 nm, less than about 6 nm, less than about 4 nm, or less than about 2 nm, etc., and the layers may be of the same or different thicknesses.

Any suitable technique can be used for depositing metals, and if more than one metal is used, the techniques for depositing each of the metals may independently be the same or different. For example, in one set of embodiments, deposition techniques such as sputtering can be used. Other examples include, but are not limited to, physical vapor deposition, vacuum deposition, chemical vapor deposition, cathodic arc deposition, evaporative deposition, e-beam PVD, pulsed laser deposition, ion-beam sputtering, reactive sputtering, ion-assisted deposition, high-target-utilization sputtering, high-power impulse magnetron sputtering, gas flow sputtering, or the like.

The metals can be chosen in some cases such that the deposition process yields a pre-stressed arrangement, e.g., due to atomic lattice mismatch, which causes the subsequent metal leads to warp or bend, for example, once released from the substrate. Although such processes were typically undesired in the prior art, in certain embodiments of the present invention, such pre-stressed arrangements may be used to cause the resulting device to form a 3-dimensional structure, in some cases spontaneously, upon release from the substrate. However, it should be understood that in other embodiments, the metals may not necessary be deposited in a pre-stressed arrangement.

Examples of metals that can be deposited (stressed or unstressed) include, but are not limited to, aluminum, gold, silver, copper, molybdenum, tantalum, titanium, nickel, tungsten, chromium, palladium, as well as any combinations of these and/or other metals. For example, a chromium/palladium/chromium deposition process, in some embodiments, may form a pre-stressed arrangement that is able to spontaneously form a 3-dimensional structure after release from the substrate.

In certain embodiments, a “coating” polymer can be deposited (240 in FIG. 8), e.g., on at least some of the conductive pathways and/or at least some of the nanoscale wires. The coating polymer may include one or more polymers, which may be deposited as one or more layers. In some embodiments, the coating polymer may be deposited on one or more portions of a substrate, e.g., as a layer of material such that portions of the coating polymer can be subsequently removed, e.g., using lithographic techniques such as e-beam lithography, photolithography, X-ray lithography, extreme ultraviolet lithography, ion projection lithography, etc., or using other techniques for removing polymer that are known to those of ordinary skill in the art, similar to the other polymers previously discussed. The coating polymers can be the same or different from the lead polymers and/or the bedding polymers. In some cases, more than one coating polymer may be used, e.g., deposited as more than one layer (e.g., sequentially), and each layer may independently have a thickness of less than about 5 micrometers, less than about 4 micrometers, less than about 3 micrometers, less than about 2 micrometers, less than about 1 micrometer, less than about 900 nm, less than about 800 nm, less than about 700 nm, less than about 600 nm, less than about 500 nm, less than about 400 nm, less than about 300 nm, less than about 200 nm, less than about 100 nm, etc.

Any suitable polymer may be used as the coating polymer. In some cases, one or more of the polymers can be chosen to be biocompatible and/or biodegradable. For example, in one set of embodiments, one or more of the polymers may comprise poly(methyl methacrylate). In certain embodiments, one or more of the coating polymers may comprise a photoresist, e.g., as discussed herein.

In certain embodiments, one or more of the coating polymers can be heated or baked, e.g., before or after depositing nanoscale wires thereon as discussed below, and/or before or after patterning the coating polymer. For example, such heating or baking, in some cases, is important to prepare the polymer for lithographic patterning. In various embodiments, the coating polymer may be heated to a temperature of at least about 30° C., at least about 65° C., at least about 95° C., at least about 150° C., or at least about 180° C., etc.

After formation of the device, some or all of the sacrificial material may then be removed in some cases. In one set of embodiments, for example, at least a portion of the sacrificial material is exposed to an etchant able to remove the sacrificial material. For example, if the sacrificial material is a metal such as nickel, a suitable etchant (for example, a metal etchant such as a nickel etchant, acetone, etc.) can be used to remove the sacrificial metal. Many such etchants may be readily obtained commercially. In addition, in some embodiments, the device can also be dried, e.g., in air (e.g., passively), by using a heat source, by using a critical point dryer, etc.

In certain embodiments, upon removal of the sacrificial material, pre-stressed portions of the device (e.g., metal leads containing dissimilar metals) can spontaneously cause the device to adopt a 3-dimensional structure. In some cases, the device may form a 3-dimensional structure as discussed herein. For example, the device may have an open porosity of at least about 30%, at least about 40%, at least about 50%, at least about 60%, at least about 70%, at least about 75%, at least about 80%, at least about 85%, at least about 90%, at least about 95%, at least about 97, at least about 99%, at least about 99.5%, or at least about 99.8%. The device may also have, in some cases, an average pore size of at least about 100 micrometers, at least about 200 micrometers, at least about 300 micrometers, at least about 400 micrometers, at least about 500 micrometers, at least about 600 micrometers, at least about 700 micrometers, at least about 800 micrometers, at least about 900 micrometers, or at least about 1 mm, and/or an average pore size of no more than about 1.5 mm, no more than about 1.4 mm, no more than about 1.3 mm, no more than about 1.2 mm, no more than about 1.1 mm, no more than about 1 mm, no more than about 900 micrometers, no more than about 800 micrometers, no more than about 700 micrometers, no more than about 600 micrometers, or no more than about 500 micrometers, etc.

However, in other embodiments, further manipulation may be needed to cause the device to adopt a 3-dimensional structure, e.g., one with properties such as is discussed herein. For example, after removal of the sacrificial material, the device may need to be rolled, curled, folded, creased, etc., or otherwise manipulated to form the 3-dimensional structure. Such manipulations can be done using any suitable technique, e.g., manually, or using a machine. In some cases, the device, after insertion into matter, is able to expand, unroll, uncurl, etc., at least partially, e.g., due to the shape or structure of the device. For example, a mesh device may be able to expand after leaving the syringe.

Other materials may be also added to the device, e.g., before or after it forms a 3-dimensional structure, for example, to help stabilize the structure, to add additional agents to enhance its biocompatibility (e.g., growth hormones, extracellular matrix protein, Matrigel™, etc.), to cause it to form a suitable 3-dimension structure, to control pore sizes, etc. Non-limiting examples of such materials have been previously discussed above, and include other polymers, growth hormones, extracellular matrix protein, specific metabolites or nutrients, additional device materials, or the like. Many such growth hormones are commercially available, and may be readily selected by those of ordinary skill in the art based on the specific type of cell or tissue used or desired. Similarly, non-limiting examples of extracellular matrix proteins include gelatin, laminin, fibronectin, heparan sulfate, proteoglycans, entactin, hyaluronic acid, collagen, elastin, chondroitin sulfate, keratan sulfate, Matrigel™, or the like. Many such extracellular matrix proteins are available commercially, and also can be readily identified by those of ordinary skill in the art based on the specific type of cell or tissue used or desired.

In addition, the device can be interfaced in some embodiments with one or more electronics, e.g., an external electrical system such as a computer or a transmitter (for instance, a radio transmitter, a wireless transmitter, etc.). In some cases, electronic testing of the device may be performed, e.g., before or after implantation into a subject. For instance, one or more of the metal leads may be connected to an external electrical circuit, e.g., to electronically interrogate or otherwise determine the electronic state or one or more of the nanoscale wires within the device. Such determinations may be performed quantitatively and/or qualitatively, depending on the application, and can involve all, or only a subset, of the nanoscale wires contained within the device, e.g., as discussed herein. The connections may include, for example, anisotropic conductive films and/or surfaces having conductive inks, e.g., carbon nanotube inks.

In some embodiments, the conductive path may be “printed” directly on the medium (e.g., a biological tissue, or other soft materials such as those described herein). For example, a suitable print head may be controlled to deliver the conductive ink on the surface of the medium. The print head may be controlled, for example, using micromanipulators such as those commercially available. The width of the conductive path may also be controlled, e.g., such to be less than about 1 cm, less than about 5 cm, less than about 3 cm, less than about 1 cm, less than about 5 mm, less than about 3 mm, less than about 1 mm, less than about 0.5 mm, less than about 0.3 mm, less than about 0.1, etc.

The conductive ink printed on the medium may include any suitable conductive material. For instance, as mentioned, the conductive ink may include carbon nanotubes, silver nanoparticles, gold nanparticles, and/or other materials that are electrically conductive. More than one such material may be present in some cases. The conductive inks, in some embodiments, may be dissolved or suspended within a suitable liquid, e.g., water, saline, organic solvents (such as dichloromethane, chloroform, toluene) or the like. In some cases, the liquid is applied or “printed” onto the surface of the medium. The liquid may then be removed (e.g., through evaporation, absorption into the medium, etc.) leaving behind the conductive ink to thereby form a conductive path. In some cases, the conductive ink is chosen to have a resistivity of less than about 1 ohm m, less than about 0.5 ohm m, less than about 0.3 ohm m, less than about 0.1 ohm m, less than about 0.05 ohm m, less than about 0.03 ohm m, less than about 0.01 ohm m, etc., once deposited onto the medium.

In addition, in some embodiments, more than one such conductive may be deposited or printed. For instance, there may be 2, 3, 4, 5, 6, 7, 8, 9, 10, 11, 12, 13, 14, 15, 16, or more conductive pathways that are printed, e.g., sequentially and/or simultaneously. In certain embodiments, the conductive pathways are printed such that they do not come into contact with each other. The spacing between conductive pathways may be, e.g., less than about 1 cm, less than about 5 cm, less than about 3 cm, less than about 1 cm, less than about 5 mm, less than about 3 mm, less than about 1 mm, less than about 0.5 mm, less than about 0.3 mm, less than about 0.1 mm, etc., depending on the application.

In addition, in some cases, the connection may be protected by covering at least a portion of the conductive path with an insulating material, i.e., after printing the conductive path directly onto the surface of a medium (e.g., a biological tissue or a non-conductive polymer substrate). For example, the insulating material may be electrical insulating, and/or may prevent water from reaching the conductive path. In some cases, the insulating material may be chosen to be biocompatible or biodegradable. Non-limiting examples of potentially suitable insulating materials include silicone, dental cement or other elastomers, polymers that are biodegradable (e.g., hydrolyzable), such as polylactic acid, polyglycolic acid, polycaprolactone, etc.

The following documents are incorporated herein by reference in their entireties: U.S. Pat. No. 7,211,464, issued May 1, 2007, entitled “Doped Elongated Semiconductors, Growing Such Semiconductors, Devices Including Such Semiconductors, and Fabricating Such Devices,” by Lieber, et al.; U.S. Pat. No. 7,301,199, issued Nov. 27, 2007, entitled “Nanoscale Wires and Related Devices,” by Lieber, et al.; U.S. patent application Ser. No. 10/588,833, filed Aug. 9, 2006, entitled “Nanostructures Containing Metal-Semiconductor Compounds,” by Lieber, et al., published as U.S. Patent Application Publication No. 2009/0004852 on Jan. 1, 2009; U.S. patent application Ser. No. 10/995,075, filed Nov. 22, 2004, entitled “Nanoscale Arrays, Robust Nanostructures, and Related Devices,” by Whang, et al., published as 2005/0253137 on Nov. 17, 2005; U.S. patent application Ser. No. 11/629,722, filed Dec. 15, 2006, entitled “Nanosensors,” by Wang, et al., published as U.S. Patent Application Publication No. 2007/0264623 on Nov. 15, 2007; International Patent Application No. PCT/US2007/008540, filed Apr. 6, 2007, entitled “Nanoscale Wire Methods and Devices,” by Lieber et al., published as WO 2007/145701 on Dec. 21, 2007; U.S. patent application Ser. No. 12/308,207, filed Dec. 9, 2008, entitled “Nanosensors and Related Technologies,” by Lieber, et al.; U.S. Pat. No. 8,232,584, issued Jul. 31, 2012, entitled “Nanoscale Sensors,” by Lieber, et al.; U.S. patent application Ser. No. 12/312,740, filed May 22, 2009, entitled “High-Sensitivity Nanoscale Wire Sensors,” by Lieber, et al., published as U.S. Patent Application Publication No. 2010/0152057 on Jun. 17, 2010; International Patent Application No. PCT/US2010/050199, filed Sep. 24, 2010, entitled “Bent Nanowires and Related Probing of Species,” by Tian, et al., published as WO 2011/038228 on Mar. 31, 2011; U.S. patent application Ser. No. 14/018,075, filed Sep. 4, 2013, entitled “Methods And Systems For Scaffolds Comprising Nanoelectronic Components,” by Lieber, et al.; and Int. Patent Application Serial No. PCT/US2013/055910, filed Aug. 19, 2013, entitled “Nanoscale Wire Probes,” by Lieber, et al.

In addition, U.S. patent application Ser. No. 14/018,075, filed Sep. 4, 2014, entitled “Methods And Systems For Scaffolds Comprising Nanoelectronic Components,” by Lieber, et al., published as U.S. Patent Application Publication No. 2014/0073063 on Mar. 13, 2014; U.S. patent application Ser. No. 14/018,082, filed Sep. 4, 2013, entitled “Scaffolds Comprising Nanoelectronic Components For Cells, Tissues, And Other Applications,” by Lieber, et al., published as U.S. Patent Application Publication No. 2014/0074253 on Mar. 13, 2014; International Patent Application No. PCT/US14/32743, filed Apr. 2, 2014, entitled “Three-Dimensional Networks Comprising Nanoelectronics,” by Lieber, et al.; and U.S. Provisional Patent Application Ser. No. 61/911,294, filed Dec. 3, 2013, entitled “Nanoscale Wire Probes for the Brain and other Applications,” by Lieber, et al. are each incorporated herein by reference in its entirety.

Furthermore, U.S. Provisional Patent Application Ser. No. 61/975,601, filed Apr. 4, 2014, entitled “Systems and Methods for Injectable Devices”; and International Patent Application No. PCT/US15/24252, filed Apr. 3, 2015, entitled “Systems and Methods for Injectable Devices” are each incorporated herein by reference in its entirety. Also incorporated herein by reference in their entireties are U.S. Provisional Patent Application Ser. No. 62/201,006, filed Aug. 4, 2015, entitled “Syringe Injectable Electronics: Precise Targeted Delivery with Quantitative Input/Output,” by Lieber, et al.; and U.S. Provisional Patent Application Ser. No. 62/209,255, filed Aug. 24, 2015, entitled “Techniques and Systems for Injection and/or Connection of Electrical Devices,” by Lieber, et al.

The following examples are intended to illustrate certain embodiments of the present invention, but do not exemplify the full scope of the invention.

### EXAMPLE 1

Syringe-injectable mesh electronics with tissue-like mechanical properties and open macroporous structures is an emerging powerful paradigm for mapping and modulating brain activity. Indeed, the ultra-flexible macroporous structure has exhibited unprecedented minimal/non-invasiveness and the promotion of attractive interactions with neurons in chronic studies. These same structural features also pose new challenges and opportunities for precise targeted delivery in specific brain regions and quantitative input/output (I/O) connectivity needed for reliable electrical measurements. This example describes results that address in a flexible manner these and other points.

This example shows the development of a controlled injection approach that maintains an extended mesh structure during the injection process, while also achieving targeted delivery with about 20 micrometer spatial precision. Optical and micro-computed tomography results from injections into tissue-like hydrogel, ex vivo brain tissue and in vivo brains validate the basic approach and demonstrate its generality. This example also presents a general strategy to achieve up to 100% multi-channel I/O connectivity using an automated conductive ink printing methodology to connect the mesh electronics and a flexible flat cable, which serves as the standard “plug-in” interface to measurement electronics. Studies of resistance versus printed line width were used to identify various operating conditions, and moreover, frequency-dependent noise measurements showed that the flexible printing process yields values comparable to commercial flip-chip bonding technology. These results thereby show facile in vivo applications of injectable mesh electronics as general and powerful tool for various applications such as long-term mapping and modulation of brain activity in fundamental neuroscience through therapeutic biomedical studies.

Syringe-injectable electronics represents a paradigm-shifting approach for seamless three-dimensional (3D) integration of electronics within man-made materials and living systems, for example, for in vivo interrogation and modulation of brain activity. In particular, unlike traditional and relatively rigid implantable brain probes based on metal, silicon and 10's of micrometer thick polymer films, certain types of syringe-injectable electronics build upon a submicron thickness macroporous mesh structure with tissue-like mechanical properties. The syringe-injectable electronics may have a bending stiffness 4-6 orders of magnitude smaller than traditional implantable probes, mesh widths features on the 10 micrometer scale similar to neuronal soma and axons, or about 90% free area structure that allows for facile neuronal interpenetration. These structural and mechanical properties of syringe injectable electronics may yield minimal damage and immune response post implantation in brain tissue as well as unprecedented attractive or “neurophilic” interactions with neurons, e.g., allowing for 3D interpenetration with intact neuronal networks.

The flexibility of the mesh electronics also presents challenges associated with the injection and input/output (I/O) connection processes. For example, during syringe-assisted injection into brain tissue the mesh electronics may “crumple” due to the extremely low bending stiffness of the structure. See, e.g., FIG. 1D, right. Such crumpling can displace the recording electrodes from expected stereotaxic injection coordinates and may yield uncertainty in specific location from which signals are recorded. In addition, small syringe needle diameters may preclude injection of mesh electronics with pre-bonded I/O connectors, and the mesh thickness and flexibility can make it incompatible with conventional semiconductor bonding methods such as wire-bonding or soldering. Although anisotropic conductive film (ACF) is a widely used approach for I/O bonding of flexible electronics, it is not optimal for the mesh electronics due to the relatively high temperatures and pressures required for bonding, and difficulties in accommodating different orientations of the mesh electronics I/O pads after unfolding post-injection. Although these aforementioned challenges are not associated with conventional rigid probes, it is noteworthy that the flexibility of syringe-injected electronics does not impose forces post-injection with respect to brain tissue and thus does not yield stresses at or motion with respect to targeted sites. Moreover, the intrinsically small and flexible nature of mesh electronics eliminates the bulky vertically-protruding I/O interface typically associated with rigid brain probes.

In this example, the controlled injection elements of the stereotaxic surgery station used for in vivo brain probe implantation used a syringe pump and a motorized stereotaxic stage (FIG. 1A). The syringe pump typically injects 1× phosphate buffered solution (PBS) through a needle loaded with mesh electronics at a fixed volumetric rate (e.g., 20 to 50 mL/h), while a motorized linear translation stage withdraws the “vertical” arm of the stereotaxic stage at a constant velocity (0.2 to 0.5 mm/s) that substantially matches the ejection rate of the mesh electronics from the needle. In general, the fluid shear force that drives the mesh electronics out of needle must overcome the friction between the mesh electronics and the needle inner wall, and thus mesh structures with different designs and different needle inner diameters (IDs) will require different flow conditions to balance electronics injection and needle retraction rates necessary to achieve full extension of the mesh electronics (FIG. 1A). The I/O bonding components of this example setup, which are also compatible with the stereotaxic surgery station, includes a motorized and computer-controlled microprinter that prints conductive ink in a programmable two-dimensional (2D) pattern that links I/O pads on the mesh electronics to corresponding channel lines of the flexible flat cable (FFC) (FIG. 1B), which then provides a standard serial communication interface with the recording/control instrumentation.

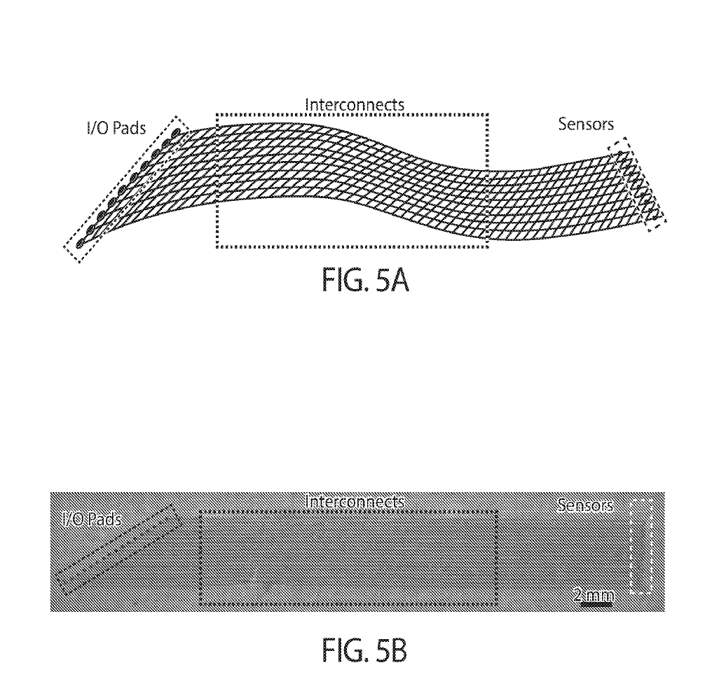

FIG. 1 shows an overview of mesh electronics injection and I/O bonding. FIG. 1A shows schematics (left, I) of the controlled injection setup for delivery of mesh electronics into a live mouse brain using a syringe pump (indicated by the arrow) and a stereotaxic frame equipped with a motorized linear translational stage, as used in this example. On the right (II), FIG. 1 shows a zoomed (dashed red box from panel I) of a mouse head, showing the extended mesh electronics inside the mouse brain after injection with needle outside the skull. FIG. 1B is a schematic showing the I/O of the mesh electronics unfolded on a flexible flat cable (FFC); electrical connections between individual channels of the mesh (left arrow) and FFC are made by printed conductive ink (right arrow). The top arrow indicates the connection of the FFC cable to external instrumentation. FIG. 1C (left, I) is an image showing mesh electronics being injected from a glass needle into 1× PBS solution. The mesh electronics expands to a size larger than the needle ID (400 micrometers) in solution. Longitudinal metal interconnect lines are prominent in the image (due to good light reflection). FIG. 1C (right, II) shows a magnified portion of mesh, highlighted by the dashed box in panel I, showing the full mesh structure. The right arrow highlights one of the longitudinal SU-8/metal interconnect/SU-8 elements, and the left arrow denotes a transverse SU-8 element (see FIG. 5 for the structure of mesh electronics). FIG. 1D is a schematic showing extended mesh electronics (left) and crumpled mesh electronics (right) post injection into dense tissue or gel.

The ultra-flexible nature of the mesh electronics, which comprises the sensors, interconnects and I/O pads (FIG. 5), is readily evident upon injection into aqueous solution (FIG. 1C), where the mesh spontaneously expands to a size substantially larger than injection needle and appears to “float” within the solution. In synthetic gels or dense tissue such as the brain, matching the mesh electronics injection and needle retraction rates may be important for achieving precise targeted delivery with a controlled and extended conformation (FIG. 1D, left) versus, for example, a crumpled conformation (FIG. 1D, right). The latter crumpled configuration yields poorly defined sensor device positions.

FIG. 5 shows the structure of syringe-injectable mesh electronics used in this example, FIG. 5A is a schematic of the mesh electronics structure, where the network corresponds to SU-8 polymer, which defines the overall mesh structure and encapsulates the metal interconnect lines in the three-layer SU-8/metal/SU-8 structure, the right dashed box highlights the sensor electrodes (dots), the midle dashed box highlights the metal interconnect lines, and the left dashed box highlights the I/O pads (circles). FIG. 5B is an optical image of a fabricated mesh electronics probe, where the left, center, and right dashed boxes highlight the sensor electrodes, the metal interconnects and the I/O pads, respectively, as in FIG. 5A.

### EXAMPLE 2

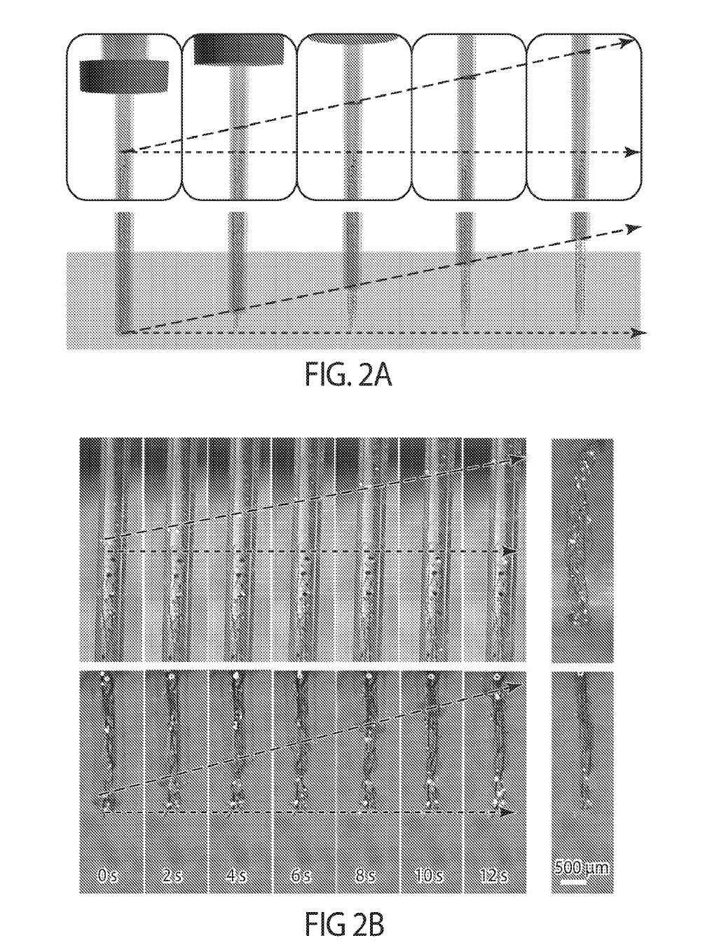

A general attribute of syringe injection is the ability to deliver materials to hidden or opaque regions, such as tissue within the brain, in a minimally invasive manner. As discussed above for this specific example of syringe injection of mesh electronics, it is important to have visual guidance and feedback to match mesh injection/needle retraction rates to ensure the mesh electronics is delivered into a targeted brain region with extended conformation. Because direct visualization of mesh electronics inside an opaque material such as the brain cannot be carried out during injection, this example shows a general method based on visualization and real-time tracking of the upper I/O end of the mesh electronics in the field of view (FoV) of an eyepiece camera (FIG. 2A). The FoV method dictates that, if the mesh electronics remains fully extended along the longitudinal direction during the injection process without displacement, then the absolute spatial location of the mesh electronics remains the same and an image of the upper end of the mesh should be fixed in the camera FoV. In other words, precisely targeted delivery of the mesh sensor electrodes can be achieved by ensuring that the mesh stays stationary in the FoV while the needle moves upwards in the FoV (FIG. 2A, top). Correspondingly, although the bottom end of the mesh electronics remains invisible to the operator, the mesh remains stationary in the injected medium with sensing electrodes at the predefined target positions (FIG. 2A, bottom), while the needle is retracted.

FIG. 2 shows field-of-view (FoV) controlled delivery of mesh electronics. FIG. 2A shows schematics illustrating controlled injection by the FoV method. The top row shows the mesh I/O pads remain stationary (level arrow) within the FoV (box) while the needle is retracted upwards (rising arrow following the black dash marked on the needle's exterior), resulting in fully extended mesh electronics structure inside the injected medium during needle withdrawal (bottom row). FIG. 2B shows a series of photographs showing the FoV injection process into 0.5% agarose hydrogel. The top row shows the needle moving upwards (rising dashed arrow) with the mesh I/O pads remaining stationary (level dashed arrow) in the FoV. The bottom series of images recorded at same time points shows the same injection process of an independent mesh structure obtained at the end of the mesh electronics in the gel. Images of the lower part of the mesh electronics in the agarose hydrogel post injection are shown in the far right panels for each experiment.

The capabilities of the FoV method were explored by injecting mesh electronics in 0.5% agarose hydrogel. This composition hydrogel is a good mimic of brain tissue since both the Young's modulus and shear modulus are similar to those of brain tissue. In addition, the optical transparency of the hydrogel allows for direct imaging of injected mesh. In experiments carried out with fluid injection rates of 20 to 50 mL/h and needle retraction speeds of 0.2 to 0.5 mm/s, it was possible to meet the stationary FoV conditions (top, FIG. 2B) as evidenced by the stationary I/O pads (level dashed arrow, top, FIG. 2B) as the needle was withdrawn at a constant speed (rising dashed arrow). A video of the injection highlights the dynamic balance of the injection/retraction rates for the full length of the process. An independent balanced injection/retraction rate experiment with the camera set to image the mesh injected in the hydrogel (bottom, FIG. 2B) reveals that the bottom edge of the mesh electronics remained stationary (level dashed arrow, bottom, FIG. 2B) as the needle was withdrawn upwards (rising dashed arrow). A video of the injection spotlights the good stability of the mesh end during this dynamic injection/retraction process. Last, both injections resulted in fully extended mesh structures in the longitudinal direction at the completion of the injection process (far right image panels, FIG. 2B).

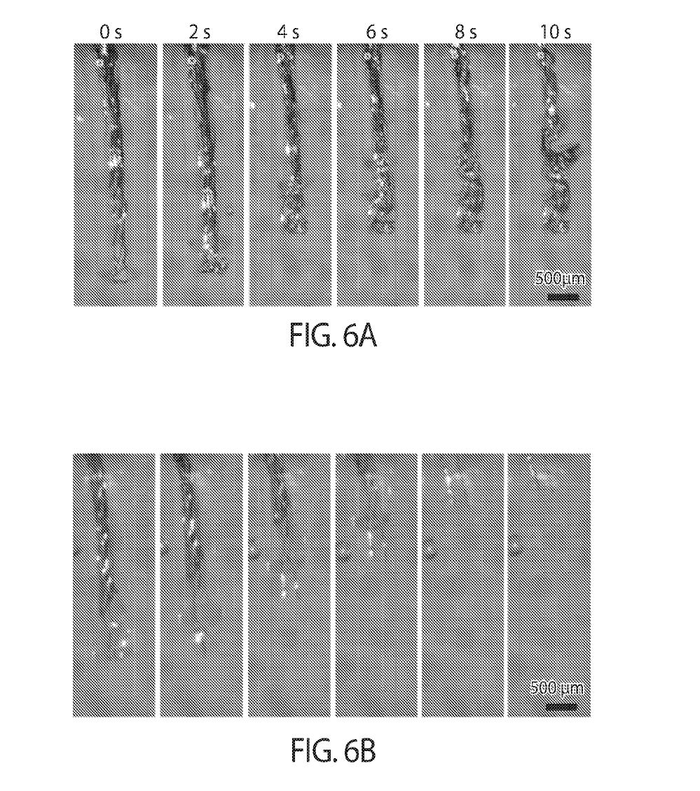

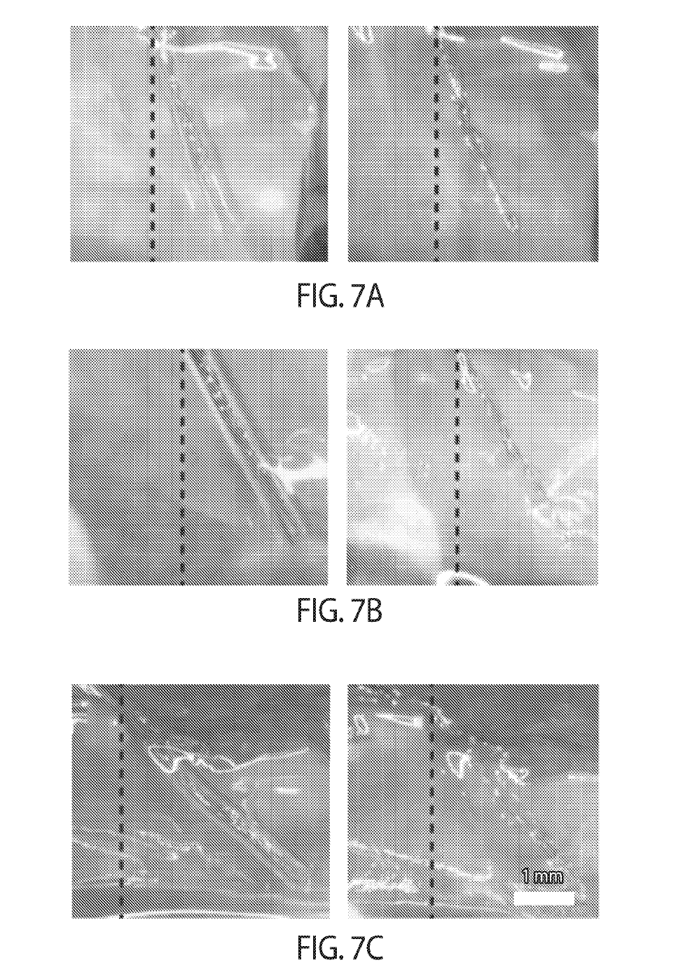

Analyses of the above results and additional experiments highlight several important points. The total volume of liquid delivered into the hydrogel during injection of an about 5 mm length of mesh electronics is typically 10 to 100 microliters. Significantly, this volume was similar to the volume of liquid introduced during intracranial injection of virus vectors and enzymes, 1 to 100 microliters. The final positioning precision of the mesh electronics in hydrogel measured during the injection process from the camera images was about 20 micrometers from the original target coordinates at t=0 s (see below for details). This relatively small positioning uncertainty suggests that the FoV injection approach can achieve precise targeted delivery of mesh electronics with tolerance smaller than the thickness of key subfields/layers of the mouse brain: for example, the CA-1 subfield of the hippocampus is about 620 micrometrs thick, the CA-3 subfield is about 230 micrometrs thick, and cortical layer V is about 300 micrometrs thick. Also, the importance of matching mesh injection/needle retraction rates was confirmed by control experiments (FIG. 6). Specifically, when the rate of needle retraction was substantially slower than injection, crumpling of the ultra-flexible mesh electronics was observed (FIG. 6A), and when the retraction substantially exceeded the injection rate, the mesh was displaced upwards from the initial targeted position during injection (FIG. 6B). Also, studies of FoV injection into tissue-like hydrogel for angles up to 45-degrees off vertical (FIG. 7) demonstrated similar targeting capabilities as vertical injection, and thus showed that controlled targeted delivery to brain regions that are typically difficult to access using vertical injection alone may be accessible with this approach.

FIG. 6 shows injection processes with mismatched injection rate and needle retraction speed. FIG. 6A shows a time course white-light optical photographs of the mesh electronics injection process when the needle is withdrawn at a speed substantially slower than the injection rate, resulting in crumpled mesh electronics structure and inaccurate delivery of mesh electrodes into the medium. FIG. 6B shows time course photos of the mesh electronics injection process when the needle is withdrawn at a speed substantially faster than the injection rate, resulting in partial withdrawal of the mesh electronics structure from the medium. In both figures, the medium was 0.5% (wt/vol %) agarose hydrogel.

FIG. 7 shows controlled injection of mesh electronics at different angles. White-light optical photographs are shown for controlled injections of mesh electronics at 15° (FIG. 7A), 30° (FIG. 7B) and 45° (FIG. 7C) to normal direction (black dashed lines) before (left) and after (right) injection. The medium in all of the experiments was 0.5% (wt/vol %) agarose hydrogel.

### EXAMPLE 3

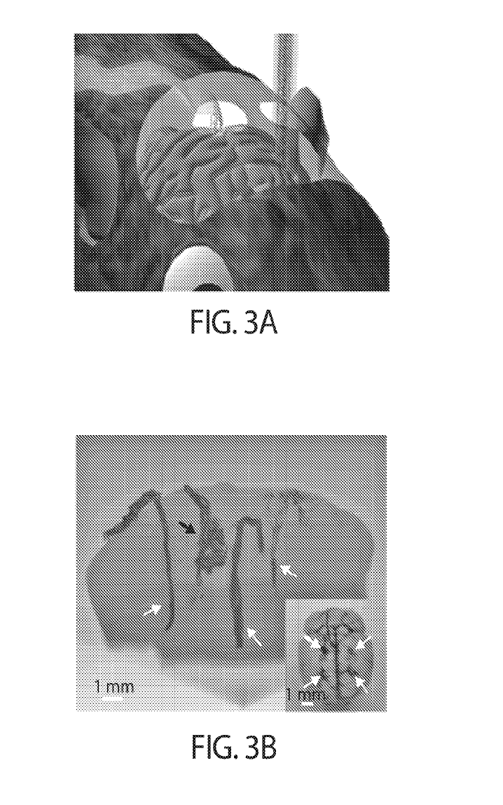

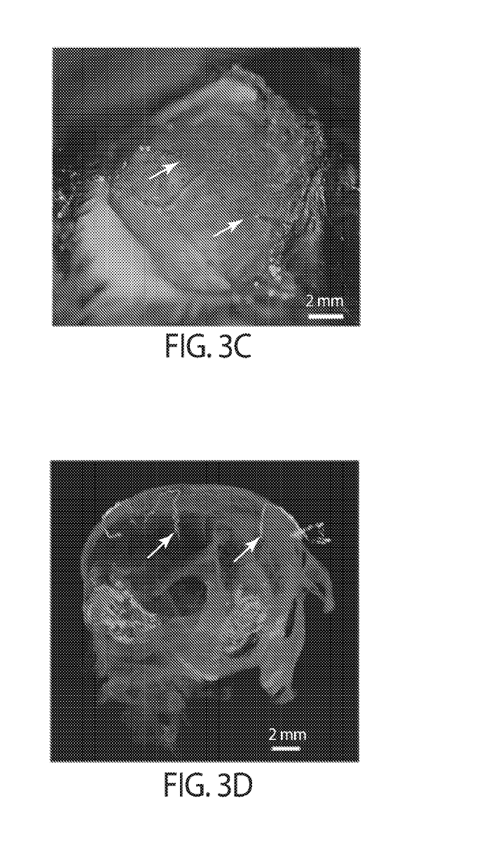

This example applies the FoV method to investigate the potential for controlled injection of mesh electronics into opaque ex vivo fixed brain tissue and in vivo live mouse brain. A schematic for in vivo injection (FIG. 3A) emphasizes the general experimental protocol of making two or more mesh injections at distinct sites prior to analysis. Four mesh electronics samples were injected at different sites in ex vivo brain tissue, where three injections were using the balanced FoV method and one was injected manually, i.e., as a control. Because the opaque nature of the brain tissue precluded direct optical imaging, micro-computed tomography (micro-CT; see below) was used to visualize the mesh electronics structure post-injection, where the high X-ray attenuation contrast of metal interconnects compared to tissue allows for clear contrast of the mesh electronics.

A 3D reconstruction of the ex vivo mouse brain following the above mesh injections (FIG. 3B) reveals several interesting points. The mesh electronics injected by the balanced FoV approach exhibited the desired fully extended morphology (light arrows). The manually injected sample showed a crumpled structure (dark arrow) in the brain tissue. It was not possible to distinguish these differences in internal morphology by optical visualization of the exterior of the brain (FIG. 3B inset), as all of these injections produced minimal visual damage/bleeding.

In addition, in vivo injection of two mesh electronic structures into the left and right cerebral hemispheres was carried out using the balanced FoV controlled injection setup under a stereotaxic stage and through pre-drilled holes in the cranial bone (FIG. 3C, see below). Analysis of the 3D reconstructed micro-CT obtained post-injection (FIG. 3D) demonstrates the fully extended mesh morphology positioned at the chosen brain coordinates for both injected mesh electronics samples. The capability to achieve well-controlled mesh electronics injections into ex vivo whole mouse brains and in vivo live mouse brains highlight several points. Synchronized and balanced mesh injection and needle retraction, which were difficult to achieve in manual injections, allowed the mesh electronics to be extended and kept stationary with respect to the optically-opaque brain tissue. Micro-CT imaging verified the effectiveness of the FoV method by proving the extended morphology of injected mesh electronics and the precise positioning within the brain using the stereotaxic stage.

FIG. 3 shows blind injection of mesh electronics using the FoV method into brain tissue. FIG. 3 3A is a schematic showing blind injection of multiple mesh electronics samples into the brain of a live mouse. The mesh electronics on the left is already injected with its I/O (left) unfolded on the skull, while the mesh electronics on the right is in the middle of the injection process. FIG. 3B is a micro-CT reconstructed image of an ex vivo mouse brain (gray) blind-injected with 4 mesh electronics samples. Inset is white-light optical image of the brain surface. Dark arrows indicate the mesh electronics injected manually, and light arrows indicate the mesh electronics delivered via the balanced FoV controlled injection process. FIG. 3C is a white-light optical photograph showing live mouse skull following blind injection of two mesh electronics samples (indicated by arrows), where both were injected using the balanced FoV controlled injection method. FIG. 3D shows a micro-CT reconstructed image of the same mouse head in FIG. 3C, showing the extended mesh electronics structures (indicated by arrows) inside the skull (gray).

### EXAMPLE 4

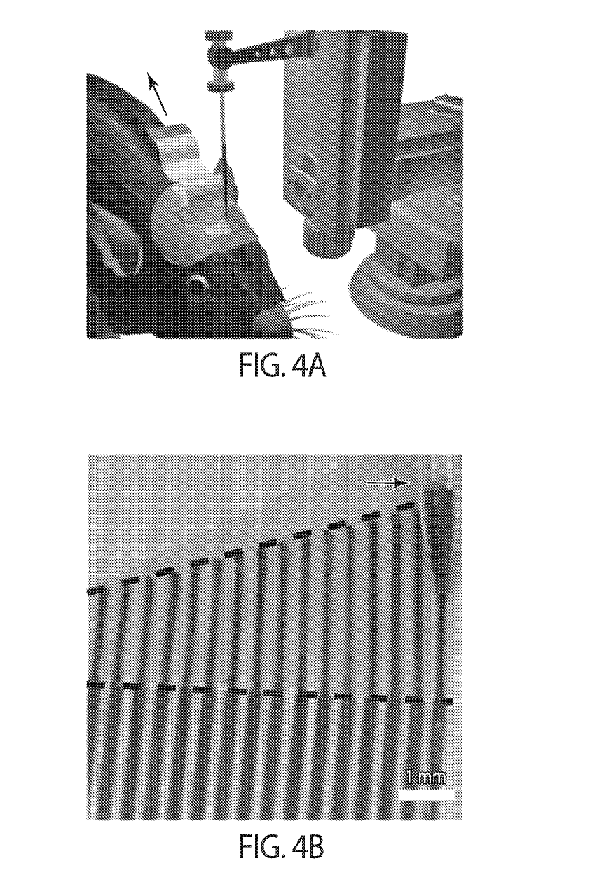

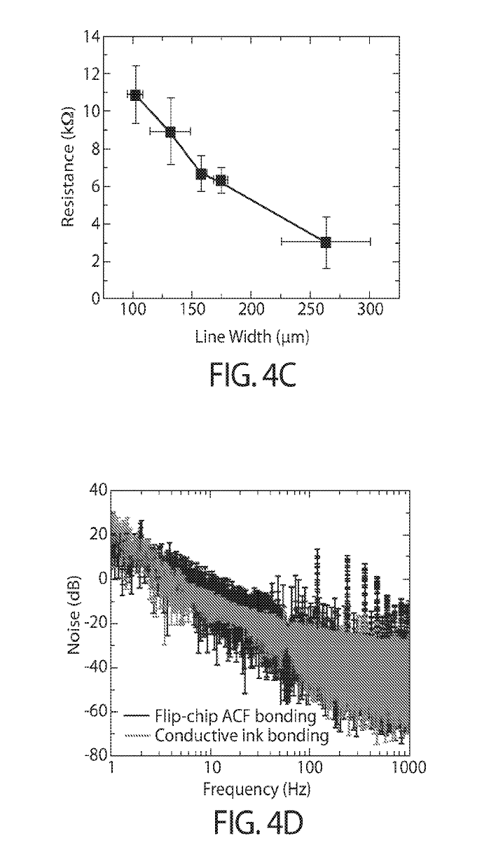

This example illustrates quantitative I/O connectivity of the multiplexed mesh electronics through an automated conductive ink printing method (FIG. 4A). The conductive ink used in this example comprised surfactant-solubilized carbon nanotubes (CNT) in an aqueous solution (see below). The CNT suspension was loaded into a glass capillary tube with a tapered tip (ID=150 micrometer) and subsequently printed as droplets on the FFC surface (FIG. 4B). For a 16-channel mesh electronics structure with recording electrodes injected into dense tissue as described above, the I/O pads at the other end of the mesh structure (FIG. 5) were unfolded on the surface of an FFC interface cable to expose the electrical connection pads for all 16 channels of the mesh electronics. The spatial coordinates of the mesh I/O pads and all FFC electrodes were taken as inputs into the automated microprinter, which then computed the shortest path for each connection and carried out conductive ink printing to make electrical connections between all available channels in the mesh and the FFC with a bonding yield of 100% (FIG. 4B). The typical time scale for unfolding the I/O pads on the FFC cable was about 10 min, and that for completing the 16-channel I/O connection through conductive ink printing was about 30 min.

FIG. 4 shows conductive ink printing for I/O connectivity. FIG. 4A is a schematic showing the automated conductive ink printing approach used to achieve high-yield I/O bonding of the mesh electronics to an FFC cable. The arrow highlights the connection from the FFC to external recording instrumentation. FIG. 4B is a white-light optical image showing all 16 channels in the mesh electronics (lined-up along the top dashed line) bonded to the 16 metal lines of the FFC cable (lower dashed line) by the conductive ink printing method, where the printed CNT lines are between the two red dashed lines. The arrow indicates the glass capillary tube loaded with conductive ink. FIG. 4C shows the resistance of conductive ink printed lines as a function of line width. All lines are printed to a total length of 5 mm. FIG. 4D shows noise spectra for mesh electronics sensor electrodes immersed in 1× PBS solution and recorded following bonding of mesh electronics I/O by standard ACF bonding (black curve) and the conductive ink printing (gray curve) methods.

The resistance of the printed CNT lines was characterized using four-point measurements as a function of line width for a fixed length of 5 mm, where the 5 mm limit was longer than lines typically used. These results (FIG. 4C) illuminate several points. The resistance of the printed CNT lines decreased as a function of line width with fixed length as expected with an estimated resistivity of (1.04+/−0.15)×10⁻² ohm m. From these data, a typical CNT line with average width of ˜150 micrometers and an average length of ˜3.5 mm was estimated to have a resistance of about 4.2 kilohms, which is much smaller than the typical interface impedance, 100 to 1000 kilohms) between metal sensing electrodes and physiological solution.

To further validate the utility of the conductive ink printing method, the noise spectra recorded from mesh electronics sensor elements were compared following bonding by standard flip-chip anisotropic conductive film (ACF) bonding, which is the standard method for flexible electronics, and the conductive ink printing method discussed herein. Notably, these data shown in FIG. 4D exhibit comparable noise-frequency dependence and thus validate our new approach.

In conclusion, these examples show controlled injection and conductive ink printing techniques to address the challenges associated with the ultra-flexible nature of syringe-injectable electronics. Controlled injection was achieved by balancing the electronics injection and the needle retraction rates, resulting in a mesh electronics structure that remains stationary and fully extended in the dense medium, thus allowing for targeted delivery of mesh electronics in any specific brain region with about 20 micrometer targeting precision. Optical and micro-CT imaging results from injections of mesh electronics into tissue-like hydrogel, ex vivo brain tissue and in vivo brains demonstrate the FoV controlled injection as a general method to achieve precise targeted delivery of mesh electronics without crumpling or displacement during injection. In addition, up to 100% I/O connectivity was demonstrated using computer-controlled hands-free conductive ink printing, which allows for customized patterns to accommodate different orientations of the mesh electronics I/O pads and pre-positions of FFC interface. Notably, frequency-dependent noise measurements show that this conductive ink printing process was comparable to commercial flip-chip bonding technology. These advances in controlled injection and I/O bonding of the mesh electronics together with previous studies showing minimal or the absence of chronic tissue response now open up many opportunities for chronic brain recording using injectable electronics, including elucidating changes in neural circuits as a function of learning and neuropathologies.

### EXAMPLE 5

This example illustrates additional materials and methods used in some of the above examples.

Fabrication of Injectable Mesh Electronics. The geometrical design of injectable mesh electronics is similar to those discussed in Int. Pat. Apl. Ser. No. PCT/US15/24252, incorporated herein by reference in its entirety. Some parameters are as follows: total width W=4 mm, longitudinal ribbon width w₁=20 micrometers, transverse ribbon width w₂=20 micrometers, angle between longitudinal and transverse ribbons α=45°, longitudinal spacing L₁=333 micrometers, transverse spacing L₂=250 micrometers, metal interconnect line width wₘ=10 micrometers and total number of channels N=16. Steps used in the fabrication of the mesh electronics are given as follows: (i) A 100 nm layer of Ni, which was used as the sacrificial layer, was thermally evaporated (Sharon Vacuum, Brockton, Mass.) onto the pre-cleaned Si wafer (n-type 0.005 ohm cm, 600 nm thermal oxide, Nova Electronic Materials, Flower Mound, Tex.). (ii) The Si wafer was spin-coated with 500 nm negative photoresist SU-8 (SU-8 2000.5; MicroChem Corp., Newton, Mass.) and pre-baked at 65° C. on a hot plate for 1 min and then transferred to a 95° C. hot plate for 4 min, before photolithography (PL) patterning (ABM mask aligner, San Jose, Calif.). The exposed SU-8 photoresist was post-baked at 65° C. for 3 min and 95° C. for 3 min. (iii) After post-baking, the SU-8 photoresist was developed (SU-8 Developer, MicroChem Corp., Newton, Mass.) for 2 min, rinsed with isopropanol, and hard-baked at 185° C. for 1 h. (iv) Subsequently, the wafer was spin-coated with MCC Primer 80/20 and LOR 3A lift-off resist (MicroChem Corp., Newton, Mass.), and baked at 185° C. for 5 min, followed by spin-coating Shipley 1805 photoresist (Microposit, The Dow Chemical Company, Marlborough, Mass.), which was baked at 115° C. for 5 min. The resist was patterned by PL and developed (MF-CD-26, Microposit, The Dow Chemical Company, Marlborough, Mass.) for 90 s. (v) A 1.5-nm Cr layer and a 100-nm thick Au layer were deposited by electron-beam evaporation (Denton Vacuum, Moorestown, NJ) followed by a lift-off (Remover PG, MicroChem Corp., Newton, Mass.). (vi) Steps iv and v were repeated for lithographically patterning and depositing the Pt sensing electrodes (Cr: 1.5 nm, Pt: 50 nm). (vii) Steps ii and iii were repeated for lithographically patterning the top SU-8 layer, which serves as the top encapsulating/passivating layer. (viii) The Si wafer with fabricated mesh electronics was transferred to a Ni etchant solution comprising 40% FeCl₃:39% HCl:H₂O=1:1:20 to release the mesh electronics from the fabrication substrate. Released mesh structures were rinsed with deionized (DI) water, transferred to an aqueous solution of poly-D-lysine (PDL, 1.0 mg/ml, MW 70,000-150,000, Sigma-Aldrich Corp., St. Louis, Mo.) for 24-48 h, and then transferred to 1× phosphate buffered saline (PBS) solution (HyClone™ Phosphate Buffered Saline, Thermo Fisher Scientific Inc., Pittsburgh, Pa.).

Controllable Injection into Dense Materials and Biological Tissues. Loading Injectable Mesh Electronics into Glass Needles. Glass capillary needles (Drummond Scientific Co., Broomall, Pa.) with inner diameter (I.D.) of 400 micrometers and outer diameter (O.D.) of 650 micrometers were used for injection tests. To load the free-standing mesh electronics, the glass needle was inserted in a micropipette holder (Q series holder, Harvard Apparatus, Holliston, Mass.), which was connected to a 1 mL syringe (NORM-JECT®, Henke Sass Wolf, Tuttlingen, Germany) through a polyethylene intrademic catheter tubing (I.D. 1.19 mm, O.D. 1.70 mm, Becton Dickinson and Company, Franklin Lakes, N.J.). The syringe was used to manually draw the mesh electronics into the glass needle.

Preparation of Hydrogel. 0.5 g agarose (SeaPlaque® Lonza Group Ltd., Basel, Switzerland) was mixed with 100 mL DI water in a glass beaker. The beaker was covered with a piece of aluminum foil (Reynolds Wrap® Reynolds Consumer Products, Lake Forest, Ill.) to prevent evaporation and heated at boiling on a hot plate until the solution was clear; the final mass concentration was about 0.5%. The solution was allowed to naturally cool to room temperature where it existed as a hydrogel with mechanical properties similar to those of dense brain tissue.

Vertebrate Animal Subjects. Adult (25-35 g) male C57BL/6J mice (Jackson Laboratory, Bar Harbor, Me.) were used as vertebrate animal subjects in this study. Animals were group-housed on a 12 h:12 h light:dark cycle in the Harvard University's Biology Research Infrastructure (BRI) and fed with food and water ad libitum as appropriate.

Preparation of Ex vivo Mouse Brains. C57BL/6J mice were euthanized via intraperitoneal injection of Euthasol (Virbac Corporation, Fort Worth, Tex.) at a dose of 270 mg/kg body weight in accordance with the recommendations of the Panel on Euthanasia of the American Veterinary Medical Association. After euthanasia, mice were decapitated and brains were removed from the skull and placed in 4% formaldehyde for 24 h for fixation. Excess formaldehyde was removed by rinsing the fixed brain in 1× PBS for 24 h and the brain tissue was stored in fresh 1× PBS solution before controllable mesh electronics injection tests.

Controlled Injection of Mesh Electronics into Hydrogel and Ex vivo Mice Brains. Either 0.5% agarose hydrogel as a brain tissue mimic or the ex vivo fixed brain tissue was placed in a petri dish. The glass needle loaded with mesh electronics was inserted in the micropipette holder, which was connected to a 5 mL syringe (Becton Dickinson and Company, Franklin Lakes, N.J.) through a polyethylene intrademic catheter tubing (I.D. 1.19 mm, O.D. 1.70 mm). The 5 mL syringe was pre-filled with 1× PBS and mounted on a syringe pump (PHD 2000, Harvard Apparatus, Holliston, Mass.). The micropipette holder was mounted on a stereotaxic stage equipped with a motorized linear translation stage (860A motorizer and 460A linear stage, Newport Corporation, Irvine, Calif.) that could move the stereotaxic arm in z direction with constant preset velocity ranging from 0.05 to 0.5 mm/s. The needle was positioned at the surface of the 0.5% hydrogel or the ex vivo fixed mouse brain samples, and liquid was injected through the mesh-loaded glass needle at a volumetric flow rate of 10 ml/h to expel air bubbles from the entire injection system. The needle was then inserted into the injection medium to the desired depth and x-y coordinates. Controllable injection was carried out by synchronizing the syringe pump with the motorized linear translation stage, with a typical liquid injection rate of 20-50 mL/h and a typical translational stage retraction velocity of 0.2-0.5 mm/s. The volumetric flow rate and the needle retraction velocity were adjusted such that the upper part of the mesh electronics, which was visualized through an eyepiece camera (DCC1240C, Thorlabs Inc., Newton, N.J.), remained stationary in the field of view (FoV) of the camera. Typical solution volumes injected into the medium with 4 mm length mesh were 10 to 100 microliters, on the same order of magnitude as the volume of liquid introduced during intracranial injection of virus vectors and enzymes in saline and artificial cerebrospinal fluid into rodent brain (ranging from 1˜100 microliters). After the glass needle was fully retracted from the injection medium, the volumetric liquid injection rate was increased to 100 mL/h to fully expel the mesh electronics from the needle onto the outer surface of the injection medium or a support used for making input/output (I/O) connections for external recording instruments. The extended morphology of the mesh in 0.5% hydrogel was verified by lowering the eyepiece camera to cover the lower part of the mesh electronics inside the transparent hydrogel. The targeting precision was estimated by tracking the motion of the bottom end of mesh electronics during the injection process using the same eyepiece camera, which had a pixel resolution of about 4.2 micrometers. For ex vivo brain tissue, the morphology of the injected mesh was verified by micro-computed tomography (micro-CT) given the optical opacity of the tissue.

Controlled In vivo Injection of Mesh Electronics into Mice Brains. For in vivo injection experiments, all metal tools in direct contact with the mice were autoclaved for 1 h and all plastic tools in direct contact with the mice were sterilized with 70% ethanol and rinsed with sterile DI water and sterile 1× PBS before use. Mesh electronic samples were sterilized by 70% ethanol followed by rinsing with sterile DI water and transferring to sterile 1× PBS. C57BL/6J mice were anesthetized by intraperitoneal injection of a mixture of 75 mg/kg of ketamine (Patterson Veterinary Supply Inc., Chicago, Ill.) and 1 mg/kg dexdomitor (Orion Corporation, Espoo, Finland). A heating pad (Harvard Apparatus, Holliston, Mass.) was set to 37° C. and placed underneath the mouse to maintain body temperature. The depth of anesthesia was monitored via the toe pinch method. In a given experiment, a mouse was placed in the stereotaxic frame (Lab Standard Stereotaxic Instrument, Stoelting Co., Wood Dale, Ill.) with two ear bars and one nose clamp used to fix the head in position. Hair removal lotion (Nair®, Church & Dwight, Ewing, N.J.) was used for depilation over the mouse head and iodophor was applied to sterilize the depilated scalp skin. A 1-mm longitudinal incision was made in the scalp, and the scalp skin was resected over the sagittal sinus of the skull, exposing a 2 mm×2 mm portion of the skull. Two 0.5 mm diameter burr holes were drilled using a dental drill (Micromotor with On/Off Pedal 110/220, Grobet USA, Carlstadt, N.J.) according to the following stereotaxic coordinates: left burr hole: anteroposterior: −1.20 mm, mediolateral: −1.25 mm; right burr hole: anteroposterior: −1.20 mm, mediolateral: +2.45 mm. The dura was carefully incised and resected using a 27-gauge needle (PrecisionGlide®, Becton Dickinson and Company, Franklin Lakes, N.J.). Sterile 1× PBS was swabbed on the surface of the brain to keep it moist throughout the surgery. The same injection process as described above was used for injection of mesh electronics into the live mouse brain through the two burr holes. Typical solution volumes injected into the medium with 4 mm length mesh were 10-100 microliters. After the two injections, the mice were euthanized via intraperitoneal injection of Euthasol at a dose of 270 mg/kg body weight and decapitated. The mouse head was fixed on a user-made stage for micro-CT imaging.

Micro-Computed Tomography. The morphologies of injected mesh electronics in opaque ex vivo brain tissue and decapitated mouse head after in vivo injection were imaged using an HMXST Micro-CT X-ray scanning system with a standard horizontal imaging axis cabinet (model: HMXST225, Nikon Metrology, Inc., Brighton, Mich.). Typical imaging parameters were 80 kV and 121 microamps (no filter) for scanning the ex vivo brain tissue, and 115 kV and 83 microamps (with a 0.1-mm copper filter for beam hardening) for scanning the decapitated mouse head with cranial bones. In both cases, shading correction and flux normalization were applied before scanning to adjust the X-ray detector. The CT Pro 3D software (ver. 2.2, Nikon-Metris, UK) was used to calibrate centers of rotation for micro-CT sinograms and to reconstruct the images. VGStudio MAX software (ver. 2.2, Volume Graphics GMbh, Germany) was used for 3D rendering and analysis of the reconstructed images. False colors were added using the VGStudio MAX software to differentiate the soft tissue, bones and the metal interconnect lines in the mesh electronics due to their different contrasts to X-ray.

Implementation and Characterization of High-Yield I/O Bonding. Preparation of Conductive Ink. Carbon nanotubes (Stock No.: P093099-11, Tubes@Rice, Houston, Tex.) were received as a slurry in toluene. The toluene was evaporated at 100° C. on a hot plate to carbon nanotube powders. 100 mg of carbon nanotube powder and 400 mg of sodium dodecylbenzenesulfonate (Sigma-Aldrich Corp., St. Louis, Mo.) were mixed with 4 mL DI water. The mixture was sonicated using a bath sonicator (Crest Ultrasonics Corp., Model 500D, Trenton, N.J.) for 1 h at its maximum power (power setting=9, power=120 W) with replacement of the sonication bath every 20 min to maintain a bath temperature<40° C. Following sonication the concentrated carbon nanotube suspension could be stored at room temperature for 3 months without significant precipitation. A brief, 5-min sonication at power setting of 9 was performed immediately prior to using as a conductive ink for I/O bonding.

I/O Bonding by Conductive Ink Printing. The carbon nanotube-based conductive ink was loaded into pulled glass capillary tube (I.D. 400 micrometer, O.D. 650 micrometer), which serves as the printer head. After pulling (Model P-97, Sutter Instrument, Calif.), the tapered tip of the glass capillary tube was ground to yield the optimal 150 micrometer I.D. The printer head was fixed with an electrode holder (Warner Instruments, Hamden, Conn.) and dipped into the freshly sonicated carbon nanotube conductive ink; capillary forces draw the conductive ink to height of about 1 cm in the printer head. The ink-loaded printer head was mounted onto a motorized micromanipulator (MP-285/M, Sutter Instrument, Novato, Calif.) controlled by a rotary optical encoder (ROE-200, Sutter Instrument, Novato, Calif.) and controller (MPC-200, Sutter Instrument, Novato, Calif.). After the I/O part of the mesh electronics was unfolded and dried to expose all I/O pads on a 16-channel flexible flat cable (FFC, PREMO-FLEX, Molex Incorporated, Lisle, Ill.), a user-written LabVIEW program was used to take the desired start position (the position of the mesh I/O pad) and end position (the position of the electrode in the FFC cable) for each channel as input coordinates and compute the minimum path between the two positions. Then the LabVIEW program drove the printer head to print the conductive ink along each computed path automatically in a ‘hopping’ motion with a typical step size of 150 micrometers. After the 16 independent connections (between mesh I/O pads FFC cable lines) each channel of the mesh electronics could be individually addressed.

Resistance Characterization of I/O Connections Using Conductive Ink. Multiple 5 mm lines were printed using the above method with widths between 80 and 300 micrometers. The resistance of each line was characterized using four-point probe resistance measurement with the inner two probes recording the voltage and the outer two recording the current on an Agilent 4156C semiconductor parameter analyzer (Agilent Technologies Inc., Santa Clara, Calif.) to minimize contact resistances.

I/O Bonding Using Anisotropic Conductive Film (ACF). The I/O part of the mesh electronics was unfolded and dried on a glass slide to expose all I/O pads. A piece of ACF (CP-13341-18AA, Dexerials America Corporation, San Jose, Calif.) with a length of 15 mm and width of 1.5 mm was placed over the I/O pads and partially bonded at 75° C. and 1 MPa for 10 s using a commercial flip-chip bonder (Fineplacer Lambda Manual Sub-Micron Flip-Chip Bonder, Finetech, Inc., Manchester, N.H.). Then an FFC cable was placed on top of the ACF, aligned with the mesh I/O pads and bonded at 165-200° C. and 4 MPa for 1-2 min.

Noise Spectrum Characterization of I/O Connections. The sensing electrodes of two identical sets of mesh electronics were immersed in 1× PBS and their I/O pads bonded using either the conductive ink printing or ACF methods. The FFC cable, which was bonded to the mesh I/O pads, was connected to an Intan RHD 2132 amplifier evaluation system (Intan Technologies LLC., Los Angeles, Calif.) through a home-made printed circuit board (PCB). Ag/AgCl electrode was used as a reference. For noise evaluation, electrical recording measurements were made with a 20-kHz sampling rate and a 60-Hz notch filter. The recorded traces were analyzed, and corresponding noise-power spectra were plotted after fast Fourier transform (FIG. 4D).

### EXAMPLE 6

This example illustrates controlled in vivo injection of mesh electronics into mouse eye for retina electrophysiology. Sterilization of tools and mesh electronics for intraocular injections in live mice was carried out as discussed herein. CD-1 mice were anesthetized by intraperitoneal injection of a mixture of 75 mg/kg of ketamine (Patterson Veterinary Supply Inc., Chicago, Ill.) and 1 mg/kg dexdomitor (Orion Corporation, Espoo, Finland). A heating pad (Harvard Apparatus, Holliston, Mass.) was set to 37° C. and placed underneath the mouse to maintain body temperature. The depth of anesthesia was monitored via the toe pinch method. In a given experiment, a mouse was placed in the stereotaxic frame (Lab Standard Stereotaxic Instrument, Stoelting Co., Wood Dale, Ill.) in left lateral recumbent position to expose its right eye. GenTeal lubricant eye gel (Novartis) was applied on the exposed eye to keep the cornea moist during surgery and prevent cataracts from developing. A sharpened capillary needle (I.D. 200 micrometers, O.D. 330 micrometers) loaded with mesh electronics was used to puncture a small hole at the lateral canthus of the eye through choroid sclera between iris and retina, while another 27-gauge hypodermic needle was used to puncture another hole at the medial canthus of the eye to release intraocular pressure during injection. A modified non-coaxial injection process from described above was employed to inject and position the mesh electronics on the retinal surface with a typical solution volume injected into the eyeball of 10-15 microliters. After the surgery, antibiotic ointment was applied copiously around the wound. Post-operative analgesic regime: After surgery, buprenorphine 0.05-0.1 mg/kg was applied to the mice every 8-12 hours for 48 hours. It is expected that after injection, electrical behavior of the retina can be monitored or controlled.

### EXAMPLE 7

This example illustrates controlled in vivo injection of mesh electronics into mouse/rat spinal cord. Sterilization of tools and mesh electronics for spinal cord injection in live mice/rats was carried out as aforementioned. The animals were anesthetized by intraperitoneal injection of a mixture of 75 mg/kg of ketamine (Patterson Veterinary Supply Inc., Chicago, Ill.) and 1 mg/kg dexdomitor (Orion Corporation, Espoo, Finland). A heating pad (Harvard Apparatus, Holliston, Mass.) was set to 37° C. and placed underneath the mouse to maintain body temperature. The depth of anesthesia was monitored via the toe pinch method. In a given experiment, a mouse/rat was placed in the stereotaxic frame (Lab Standard Stereotaxic Instrument, Stoelting Co., Wood Dale, Ill.) with two ear bars and one nose clamp used to fix the head in position. Hair removal lotion (Nair®, Church & Dwight, Ewing, N.J.) was used for depilation over the mouse back and iodophor was applied to sterilize the depilated and exposed skin. Then a small (ca. 5 mm) incision above the vertebrae of interest was created using the scalpel blade, where either forceps or surgical scissors were used to increase the size of the opening in the skin and hold the opening in place. A scalpel blade was used to gently dissociate connective fascia and underlying muscle to expose dorsal spine. A combination of scalpel blade, cotton swabs, and/or bone scraper was used to remove all overlying tissue from the dorsal laminae. Surgical scissors were used to carefully cut away tendons attached to the vertebrae on both lateral edges of the spinal column, before a 2 mm×2mm hole was drilled on the spinal column to expose the spinal tissue. The dura was carefully incised and resected using a 27-gauge needle (PrecisionGlide®, Becton Dickinson and Company, Franklin Lakes, N.J.). Sterile lx PBS was swabbed on the surface of the spinal tissue to keep it moist throughout the surgery. The same injection process as described above for brain injection was used for injection of mesh electronics into the live mouse/rat spinal cord tissue. Typical solution volumes injected into the medium with 2 mm length mesh were 10-100 microliters. After the surgery, antibiotic ointment was applied copiously around the wound. Post-operative analgesic regime: After surgery, buprenorphine 0.05-0.1 mg/kg was applied to the mice every 8-12 hours for 48 hours. It is expected that after injection, electrical behavior of the spinal cord can be monitored or controlled.

### EXAMPLE 8

Stable in vivo mapping and modulation of the same neurons and brain circuits over extended periods is critical to both neuroscience and medicine. Current electrical implants offer single-neuron spatiotemporal resolutions but face challenges of relative shear motion and chronic immune response, which yield signals shifting from targeted neurons and necessary probe position adjustments to break glial scarring. This example shows a chronic in vivo recording/stimulation platform based on ultra-flexible mesh electronics and demonstrate stable multiplexed local field potentials (LFPs) and single-unit recordings from mouse brains for at least eight months without probe repositioning. Data show almost unchanged principal components, average spike waveforms, stable firing dynamics and phase-locking of spike firings/LFP oscillations, thus suggesting robust tracking of the same neurons over this period. This platform also illustrates stable single-neuron responses to chronic electrical stimulation. The capability for long-term recording is applied to longitudinal studies of brain aging, where the firing dynamics and spike characteristics of the same individual neurons are followed, and freely behaving mice. These demonstrated advantages could open up future studies in mapping and modulating changes associated with learning, aging, and neurodegenerative diseases.

This example illustrates mesh electronics with micrometer feature sizes comparable to neuron somata and effective bending stiffness values comparable to dense neural tissue. This example demonstrates a chronic recording/stimulation mesh electronics platform that overcomes relative shear motion and chronic immune response limitations of conventional probes, and thereby allows consistent and reproducible recording from and stimulation of the same individual neurons in vivo for at least eight months.

Brain injection and recording interface for mesh electronics. Mesh electronics were fabricated using standard photolithography procedures. The overall design used an array of 16 recording or stimulation electrodes at one end addressed individually by metal interconnects, and terminated with input/output (I/O) pads at the opposite end of the mesh structure. The interconnects were sandwiched by insulating and biocompatible polymer layers, leaving only the recording/stimulation electrodes in direct contact with brain tissue. The thicknesses and widths of the mesh elements were ca. 800 nm and 20 micrometers, respectively, which yielded ultra-low bending stiffness values of ˜0.1 n Nm, comparable with neural tissue and correlated with a low immune response.

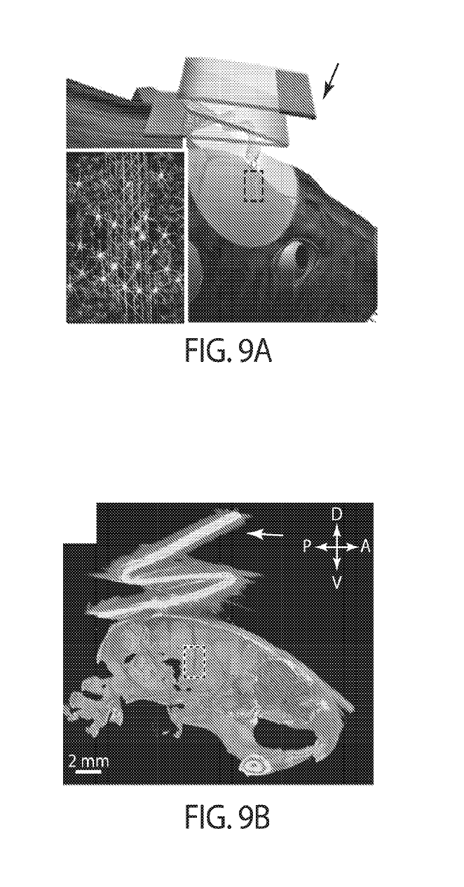

An overview of this approach (FIG. 9) highlights the flexible open mesh electronics and lightweight instrument interface. First, stereotaxic injection was used to deploy mesh electronics through a capillary needle into a targeted brain region with a positioning precision of ˜20 micrometers, an extended morphology and integration of the mesh structure with neurons (FIG. 9A). Micro-computed tomography (micro-CT) post-injection (FIG. 9B) confirmed an extended morphology along the injection direction. Second, the I/O pads of mesh electronics were unfolded onto and electrically connected to a lightweight (˜0.2 g) flexible flat cable (FFC) using printed conductive ink. The ultra-flexibility of the electronics was visualized as the rolled-up mesh “sagging” between the exit point on the brain/skull and the FFC. The FFC, which is plugged into recording instrumentation, was fixed to the mouse skull and folded to minimize its size (FIG. 9B). Finally, the positions of mesh electronics were not adjusted over the course of the chronic experiments following implantation.

FIG. 9 shows syringe-injectable mesh electronics for chronic brain mapping and modulation. FIG. 9A is a schematic showing a mouse with stereotaxically injected mesh electronics bonded through conductive ink (black lines) printing to a flexible flat cable (FFC, arrow), which is folded afterwards to minimize its profile. Inset: A zoom-in view of the dashed box showing seamless integration of the mesh electronics with neural network. The lines, white and yellow circles represent metal interconnects, recording and stimulation electrodes, respectively. FIG. 9B shows a micro computed tomography (micro-CT) image showing a lateral view of a mouse head with injected mesh electronics (dashed box) and folded FFC (arrow). Axes labeled with A, P, D, V represent anterior, posterior, dorsal and ventral anatomical directions, respectively. Scale bar: 2 mm.

### EXAMPLE 9

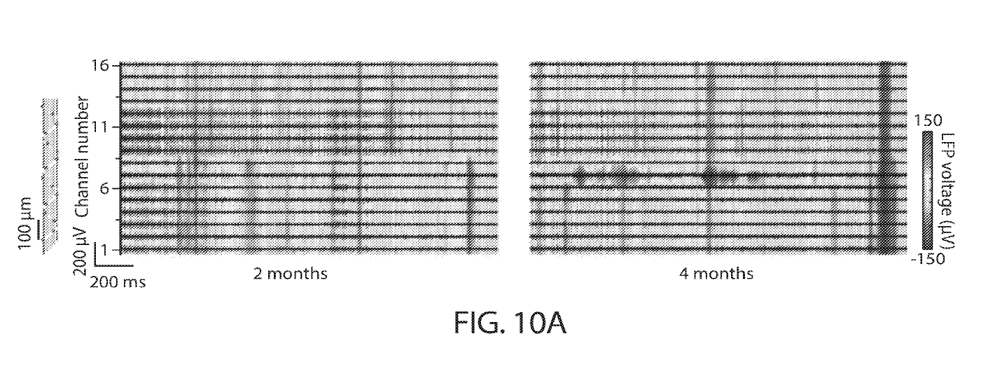

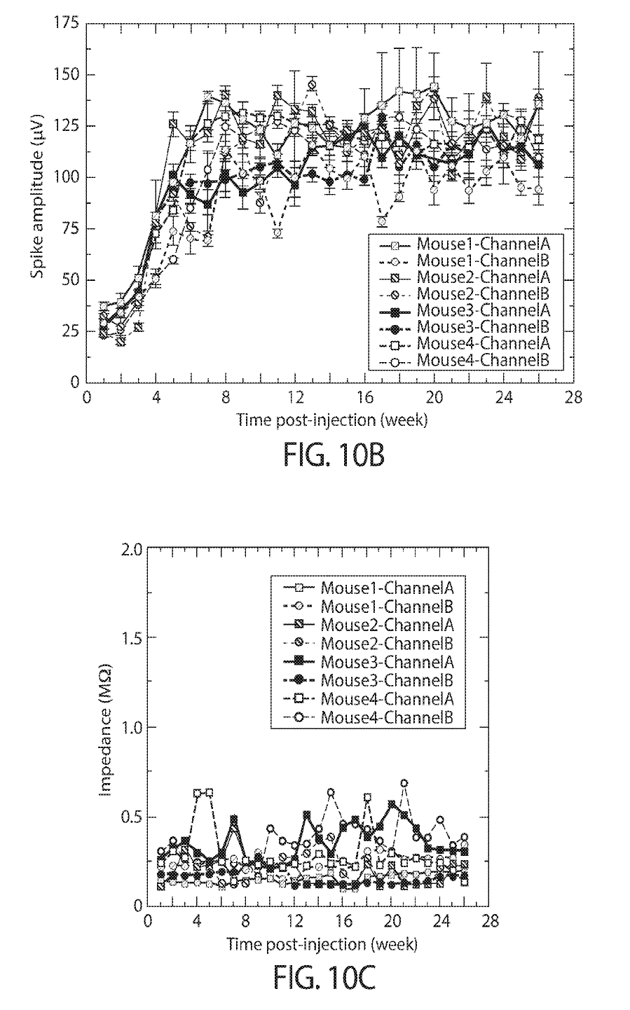

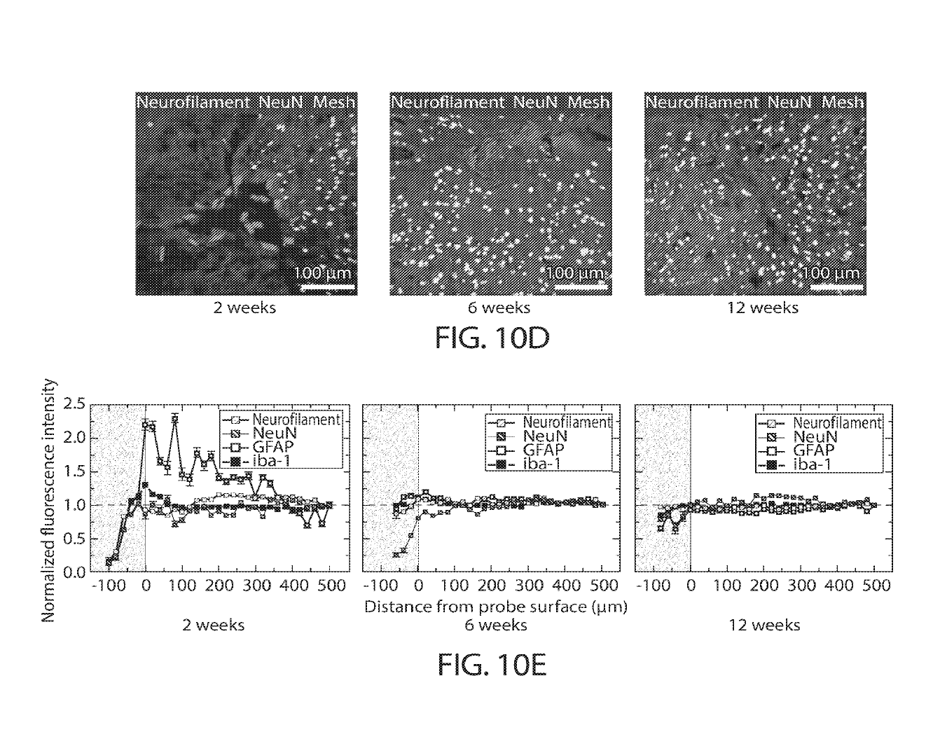

Long-term brain activity mappings at the single-neuron level. Initial long-term recording stability was assessed from 16-channel mesh electronics spanning the hippocampus (HIP) and somatosensory cortex (CTX) of a mouse. Representative multiplexed recordings from the same awake mouse at two and four months post-injection yielded well-defined LFPs in 16/16 channels with modulation amplitudes ˜300 microvolts and single-unit spikes from 14/16 channels (FIG. 10A). Focusing on single-unit spikes, it was found that different channels exhibited stable amplitudes and signal-to-noise ratios (SNRs) across this 2-month period (FIG. 2A). Cross-channel correlation maps of LFPs and single-unit spikes showed similar patterns over this time period. The constant single-unit amplitudes and similar spike firing patterns over time suggesting that these data might correspond to signals from the same neurons and neural circuits. This is addressed further below with studies extending to 8 months.

Multiplexed data recorded over at least 6-month periods from four mice showed an initial amplitude increase followed by stable spike amplitudes ˜6 weeks post-injection. Representative chronic single-unit recording traces from one electrode (Mouse4-ChannelA) highlighted several key points. First, peak-to-peak spike amplitudes increased from ˜30 microvolts (1 week) to ˜130 microvolts (2 months) and thereafter remained stable to at least 6 months post-injection, with overall firing rate approximately constant across the entire period. Two distinct clusters of sorted spikes with visually stable waveforms indicative of two neurons were observed. Waveform autocorrelation analyses showed quantitatively a large percentage of similarity both within the same recording session and across 6 months. Together these results suggest stable single-neuron recording during this extended time period. The electrode interfacial impedances (FIG. 10C) further showed relatively constant values (mean ˜300 kilohms) over time, distinct from other brain implants with reported electrode impedance fluctuations attributed to chronic immune response.

To explore the mechanism of the observed long-term single-neuron signal stability and provide insight into the short-term amplitude increase, out time-dependent histology studies were performed. Representative confocal fluorescence microscopy images of horizontal brain slices with mesh electronics at 2, 6 and 12 weeks post-injection (FIG. 10D) illustrate several important features. The 6- and 12-week images (FIG. 10D, middle and right panels) showed axonal projections and somata filling the mesh electronics interior, and quantitative analyses (FIG. 10E) demonstrated signals of axonal projections and somata close to and returning to background levels near the outer surface and interior of the mesh electronics, respectively. Notably, astrocyte and microglia data (FIG. 10E) showed signals close to and slightly below background near the outer surface and interior of the mesh, respectively.

The 2-week post-injection data provides insight into the initial increase in spike amplitude. Specifically, there was an interior depression in cell density (not evident at greater than 6 weeks) remaining from acute needle insertion damage, although quantitative analyses (FIG. 10E) demonstrated axonal projections, astrocytes and microglia in this region. These data also showed that astrocytes and microglia were somewhat enhanced up to 100-300 micrometers from the probe surface (returned to background, greater than 6 weeks). Hence, the observed amplitude increase can be associated with recovery from acute implantation damage via gradual removal of astrocytes (and microglia).

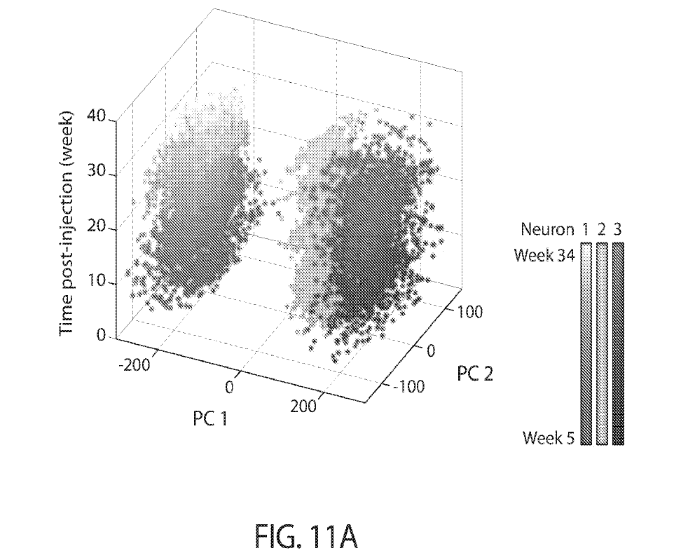

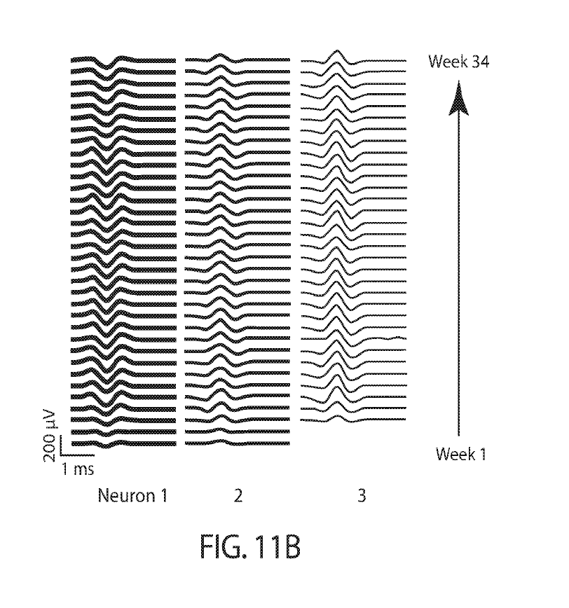

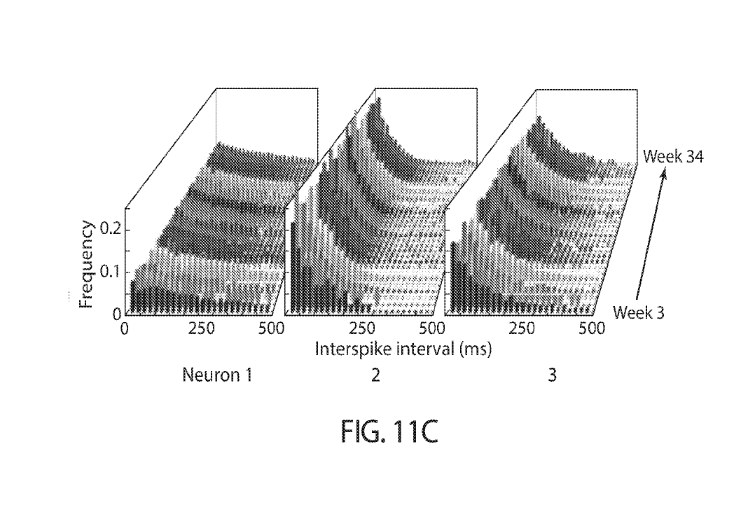

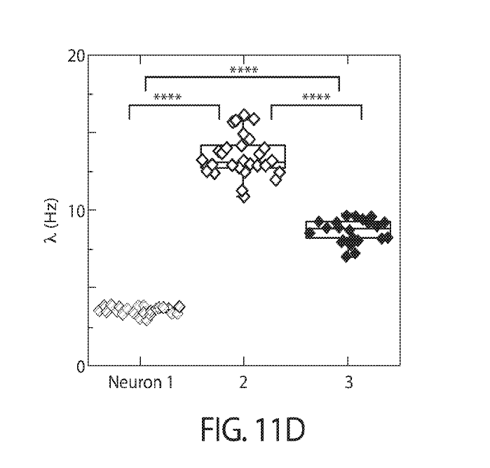

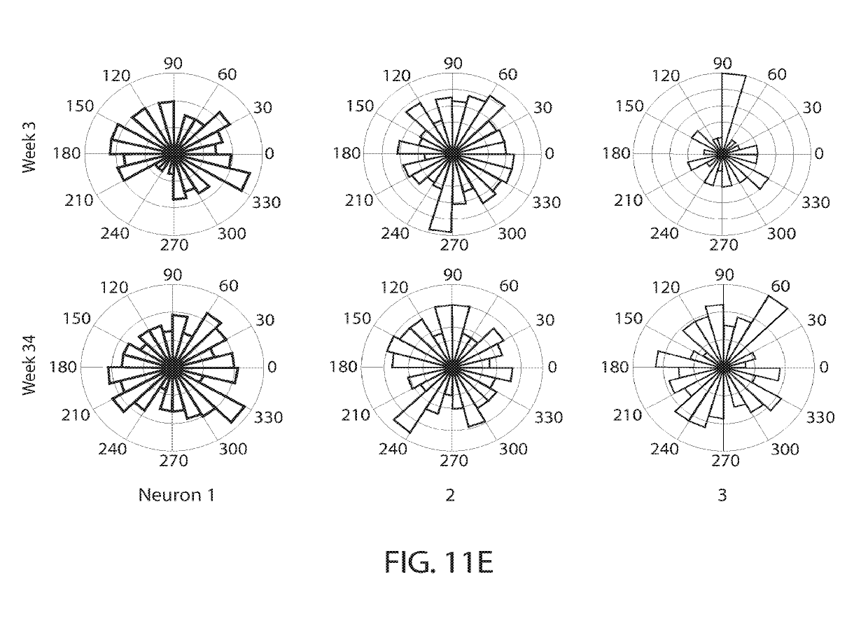

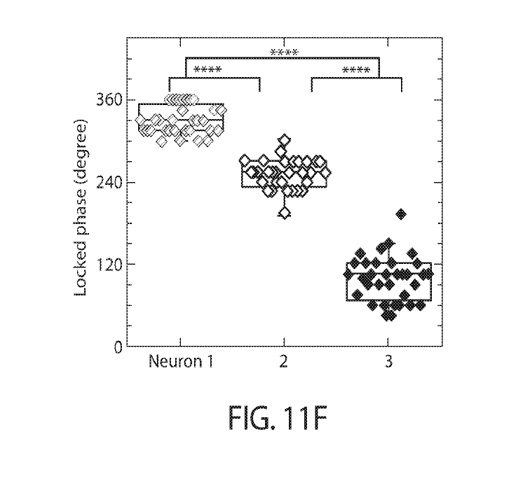

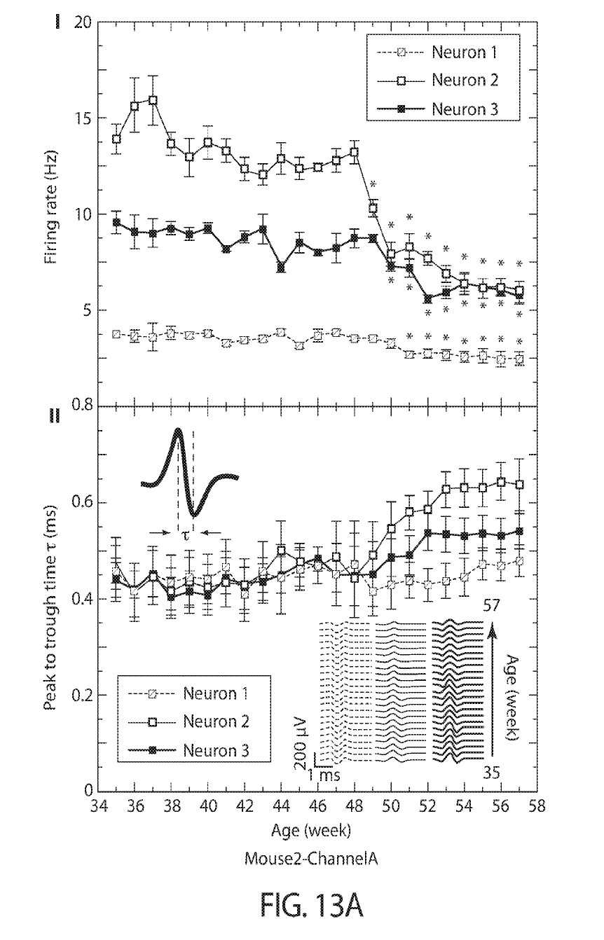

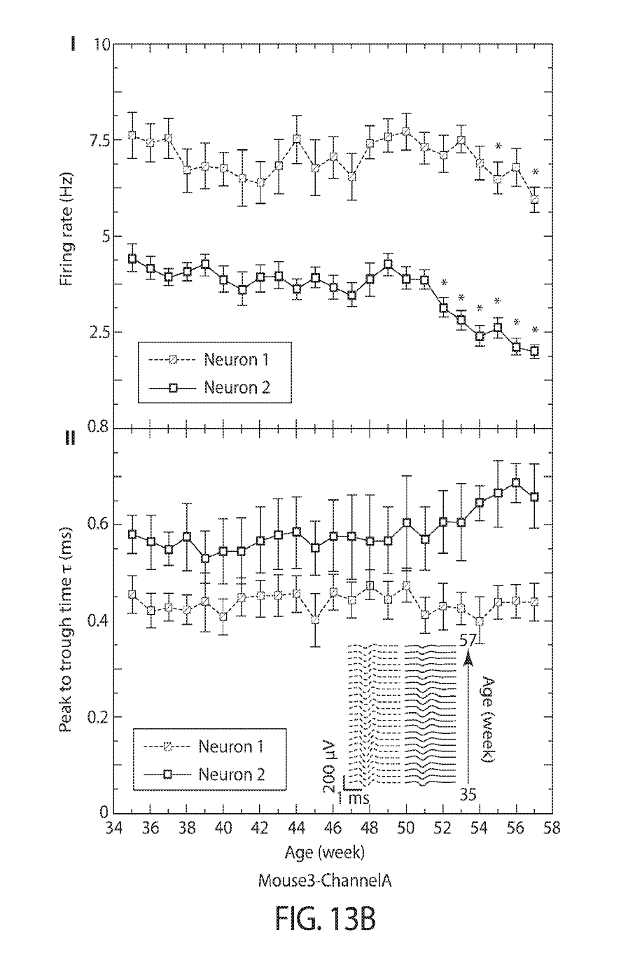

FIG. 10 shows long-term stable recording without signal degradation over six months and immunohistochemistry staining of mesh electronics/brain tissue interface. FIG. 10A shows representative 16-channel local field potential (LFP) (heat maps) with amplitudes color-coded according to the color bar on the far right and single-unit spike (traces) mapping from the same mouse at two (left) and four (right) months post-injection. The x-axes show the recording time while the y-axes represent the channel number of each recording electrode with relative position marked by red dots in the schematic (leftmost panel). FIG. 10B shows the time evolution of average spike amplitudes of representative channels from four different mice. Mouse1 represents the recordings shown in FIG. 10A with Channel A and B denoting Channel 10 and 3, respectively. Mouse2-ChannelA, B and Mouse3-ChannelA were used for analyses shown in FIGS. 11 and 13. FIG. 10C shows the time-dependent impedance values at 1 kHz of the channels shown in FIG. 10B. FIG. 10D shows immunohistochemical staining images of horizontal brain slices at 2 (left, hippocampus (HIP)), 6 (middle, cortex (CTX)) and 12 weeks (right, CTX) post-injection. Shadings correspond to neurofilaments, NeuN and mesh electronics, respectively (see keys at top). Scale bar: 100 micrometers. FIG. 10E shows neurofilament, NeuN, GFAP, and iba-1 fluorescence intensity normalized against background values (gray dashed horizontal lines) plotted versus distance from the interface. The shaded regions indicate the interior of the mesh electronics. All error bars in this figure reflect +/−1 standard error of the mean (s.e.m.).

### EXAMPLE 10

Chronic tracking of individual neurons. Statistical analyses were carried out to confirm the chronic stability of recorded neuron/neural circuit signals. Principal component analysis (PCA), which can define the number and stability of recorded single-neuron signals over time, of representative sorted spikes (FIG. 11A) showed the same three clusters with nearly constant positions in the first and second principal component plane (PC1-PC2) from 5 through 34 weeks (8 months) post-injection. Time-dependent averaged spike waveforms (FIG. 11B), waveform auto-/cross-correlation and L-ratio analyses further demonstrated good unit separation and high stability over this time period.

The individual neuron firing dynamics was characterized by the inter-spike interval (ISI) distributions for the 3 identified neurons (FIG. 11C). Notably, these ISI histograms exhibited stable and distinct distributions with characteristic 2-3 ms refractory period over 8 months. Analysis of the variance (ANOVA) on the firing parameter, λ (lambda) (reflecting neuron firing rates) obtained from exponential fits to each ISI histogram, showed a significant difference (p-value<0.0001) between any two neurons, thus confirming the same neurons were followed over this 8-month period. Similar analyses were carried out for another channel from the same mesh and one channel from another mouse. Results showed unchanged principal components, L-ratios demonstrating good unit separation, constant spike waveforms supported by auto-/cross-correlation, and stable ISI histograms and firing parameters.

To test the potential for stable recording from neural circuits, phase-locking was analyzed between single-neuron firings and LFPs with a focus on HIP data, which has been reported to show phase-locking. A Rayleigh Z-test (of the recorded data showed evidence for phase-locking at distinct angles from 3 to 34 weeks post-injection for each of the three neurons identified. Statistical analyses demonstrated stable and distinct phase-locked angles for all neurons over time with means of 330, 250 and 95 degrees (FIGS. 11E-11F), showing that mesh electronics can record from the same neural circuit over 8 months.

FIG. 11 shows consistent tracking of the same group of neurons. FIG. 11A shows the time evolution of representative single-unit spikes of Mouse2-ChannelA shown in FIG. 10 clustered by principal component analysis (PCA) over eight months post-injection. The x- and y-axis denote the first and second principal component, respectively, and the z-axis indicates post-injection time. The scale bars show the corresponding post-injection time points from 5 to 34 weeks (8 months) of the 3D PCA plots. FIG. 11B shows the time course analysis of average spike waveforms from each PCA cluster shown in (FIG. 11A). FIG. 11C shows the time evolution of inter-spike interval (ISI) histograms of each of the 3 neurons identified in (FIG. 11A) from 3 to 34 weeks. Bin size: 20 ms. FIG. 11D shows a scatter plot with analysis of variance (ANOVA) of the firing parameter (n=32 for each neuron), λ(lambda), obtained by fitting each ISI distribution profile shown in (FIG. 11C) to an exponential decay. FIG. 11E shows polar plots showing the phase-locking of single-unit spikes to theta oscillations (4-8 Hz) of LFPs in HIP for each of the 3 neurons in (FIG. 11A) at 3 and 34 weeks. FIG. 11F is a scatter plot with ANOVA test of the locked phase angle (n=32 for each neuron). For FIGS. 11D and 11F, the open rectangles and bars indicate 25/75 and 0/100 percentiles, respectively; **** indicates p value of <0.0001.

### EXAMPLE 11

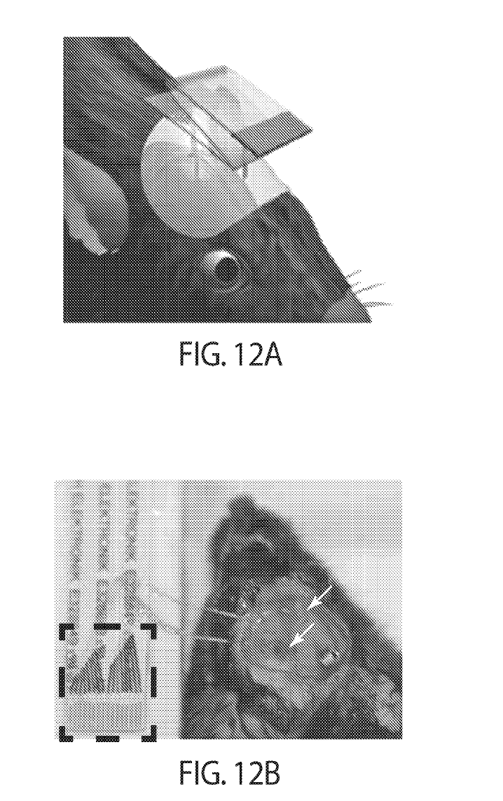

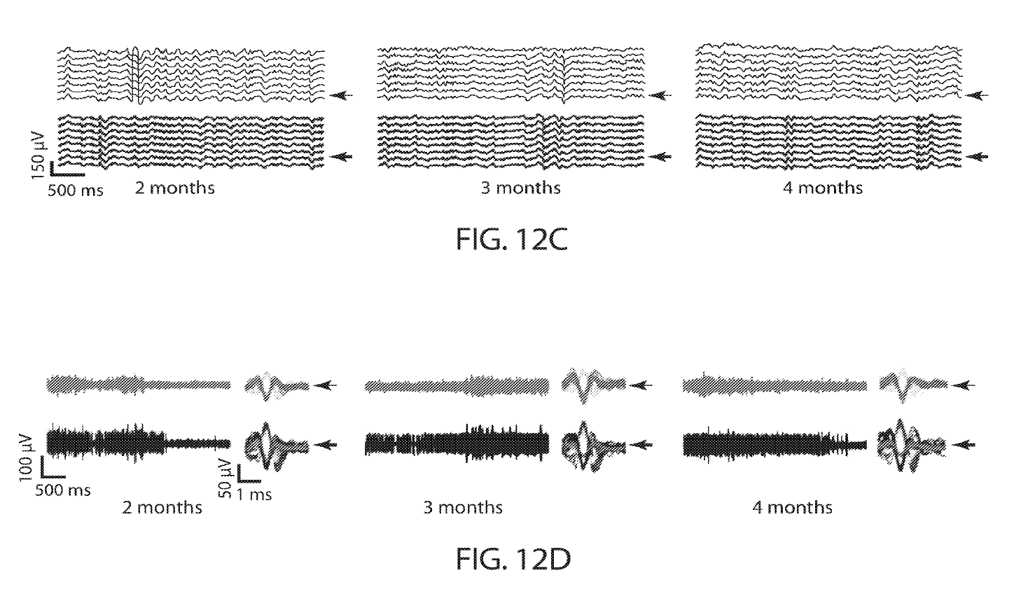

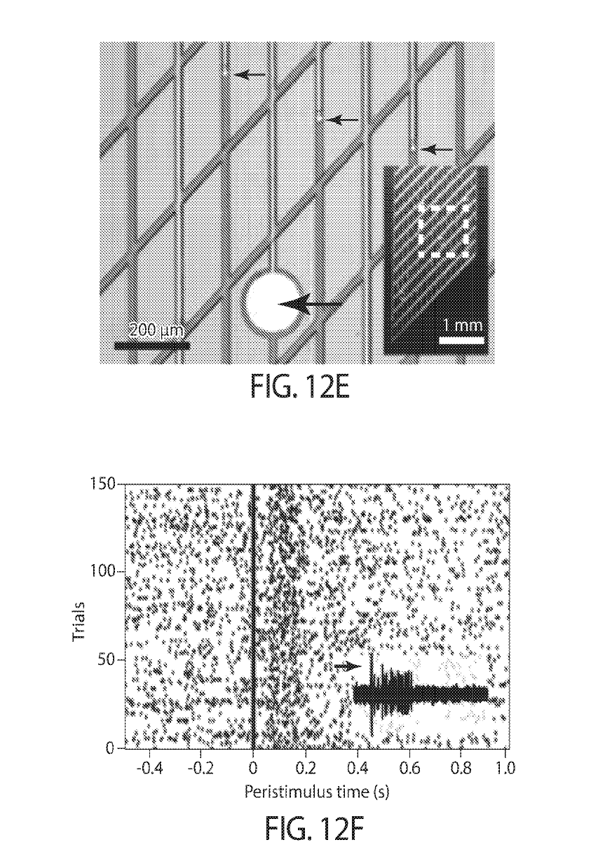

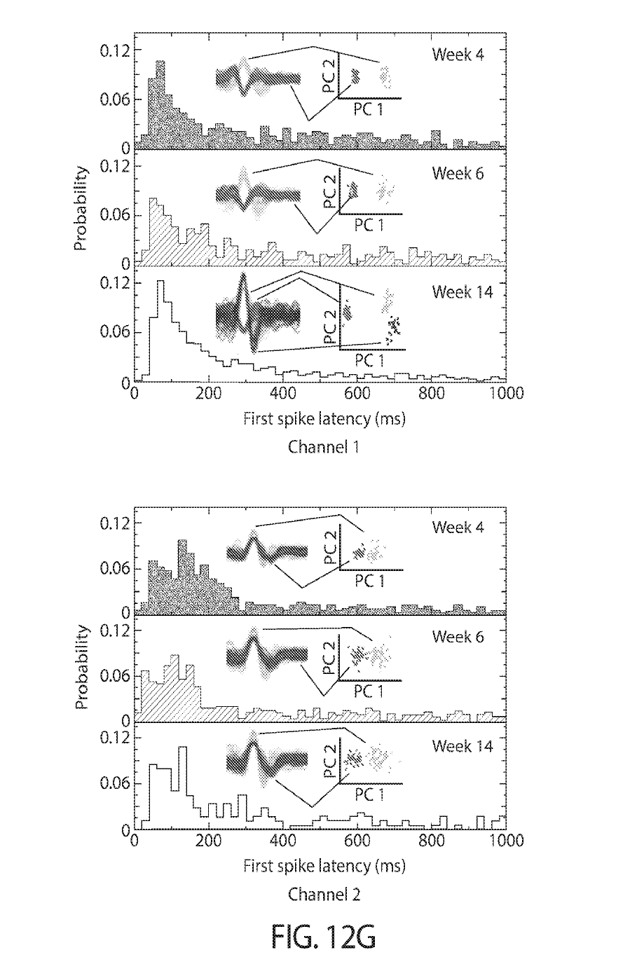

Multi-site recording from different brain regions. This example demonstrates stable chronic recording from two mesh probes injected into distinct brain regions. The I/O pads from the two meshes could be connected to the same FFC as shown schematically (FIG. 12A) and experimentally (FIG. 12B), and yielded insignificant increase in the interface weight. Multiplexed LFP recordings from mesh electronics implanted in motor CTX (upper) and HIP (lower) of opposite cerebral hemispheres showed similar modulation within a probe but distinct signals between probes (FIG. 12C). Both probes exhibited stable LFP amplitudes across at least 2 months (FIG. 12C). Representative single-unit spike traces (FIG. 12D) demonstrated consistent chronic firing dynamics, and spike-sorting (FIG. 12D) showed stable amplitudes and consistent cluster waveforms (2 identified neurons in CTX and 4 in HIP) over this time period.

FIG. 12 shows multi-site and multifunctional mesh electronics. FIG. 12A shows schematic and FIG. 12B is a photo showing two mesh electronics (white arrows) injected into different brain regions (motor CTX of the right cerebral hemisphere and HIP of the left hemisphere) of the same mouse. The two mesh probes were bonded to the same FFC (dashed box in FIG. 12B). FIG. 12C shows multiplexed LFP and FIG. 12D shows single-unit recordings along with sorted spikes from the motor CTX (upper traces) of one hemisphere and the HIP (lower traces) from the contralateral hemisphere. The three columns correspond to data recorded at 2 (left), 3 (middle) and 4 (right) months post-injection, respectively. The arrows in FIG. 12C highlight the channels corresponding to the representative spike trains shown in FIG. 12D.

Multifunctional mesh for chronic stimulation and recording. Some experiments incorporated 150-micrometer diameter low-impedance stimulation electrodes in the mesh electronics (FIG. 12E). Chronic stimulation and recording from 4 to 14 weeks post-injection highlighted certain features. First, a peristimulus spike raster plot (FIG. 12F) showed an increased firing rate following stimulation. Also, histograms of first-spike latency following stimulation for two recording electrodes (FIG. 12G, channels 1&2) exhibited consistent distributions from 4 to 14 weeks in weekly stimulation trials. Control data recorded from a second mesh implanted in the contralateral hemisphere showed no stimulation-evoked response. Third, spike-sorting and PCA analyses of channel 1 and 2 (FIG. 12g, insets) confirmed stable recording of two unique neurons for both electrodes, although a third neuron was identified at ca. 14 weeks in channel 1.

FIG. 12E is a photograph showing typical mesh electronics before releasing from substrate with unipolar stimulation electrodes (black arrow) and recording electrodes (arrows). Scale bar: 200 micrometers. Inset: Zoomed-out photograph with dashed box representing the area of FIG. 12E. Inset scale bar: 1 mm. FIG. 12F is a peristimulus raster plot showing spike events (black ticks) of 150 stimulation trials (solid line: stimulation pulse). Inset: Representative recorded spike trains from −0.15 to 0.85 s. The arrow indicates the stimulation pulse. FIG. 12G shows first spike latency distributions of stimulus-evoked firings recorded from two different electrodes (Channels 1 & 2) located in the same cerebral hemisphere but with progressively increasing distance from the stimulation electrode at 4, 6 and 14 weeks post-injection. Spike-sorting and PCA clustering results are displayed as insets.

### EXAMPLE 12

Longitudinal studies of aging at single-neuron level. The long-term recording stability with mesh electronics can enable longitudinal studies of aging-associated changes at single-neuron and neural-circuit levels. Previous research has been limited to longitudinal studies with low spatiotemporal resolution or high-resolution electrophysiology studies comparing different subjects due to chronic instability. These examples tracked the time evolution of firing dynamics and spike characteristics of individual neurons before and after two mice entered middle age, 10-12 months. These data (FIG. 13) revealed aging-associated neuronal changes. First, analyses of ISI histograms showed firing rate declines for mice aged ˜48 weeks and older with individual neurons exhibiting distinct time-dependent changes (FIGS. 13A-13B). For example, neurons 2 and 3 of Mouse2-ChannelA (FIG. 13A, I) showed decreases in firing rate starting at ˜48 weeks, while the firing of neuron 1 was relatively unaffected. Similar decreases were seen for Mouse3-ChannelA (FIG. 13B, I). Second, no systematic changes were found in electrode impedances, and histology study showed uniform distributions of neuronal somata, axonal projections, astrocytes and microglia through the mesh electronics interior at ˜1 year post-injection. These results suggested minimal or no degradation of recording electrodes, and correspondingly argue that the observed firing rate decreases are intrinsic to individual neurons. Third, quantitative analyses of sorted spike waveforms (FIGS. 13A-13B, II) revealed a neuron-specific increase of peak-to-trough time, τ (tau), starting at ˜48 weeks of mouse age; that is, neurons 2 and 3 of Mouse2-ChannelA (FIG. 13A, II) both showed increases in τ (tau), with little or no increase observed for neuron 1. These increases in single-neuron peak-to-trough times coincided temporally with the firing rate decreases, and were especially prominent for neurons with larger firing rate decreases (FIGS. 13A-13B).

FIG. 13 shows a longitudinal study of brain aging at the single-neuron level. FIGS. 13A, 13B show time evolution of average spike firing rate (I) and average peak-to-trough time τ (tau) (defined in upper left inset in (a, II)) with average spike waveforms shown as bottom right insets (II) of each neuron identified from PCA clusters from representative channels of Mouse 2 (a) and 3 (b), respectively. The x-axes show the corresponding mouse ages in all panels. The error bars in (I) show fitting errors, and * indicates statistical significant (p<0.05, double-sided t-test, n=50) decrease of firing rate compared with that at age of 48 weeks. The error bars in (II) show +/−1 standard deviation (SD).

### EXAMPLE 13

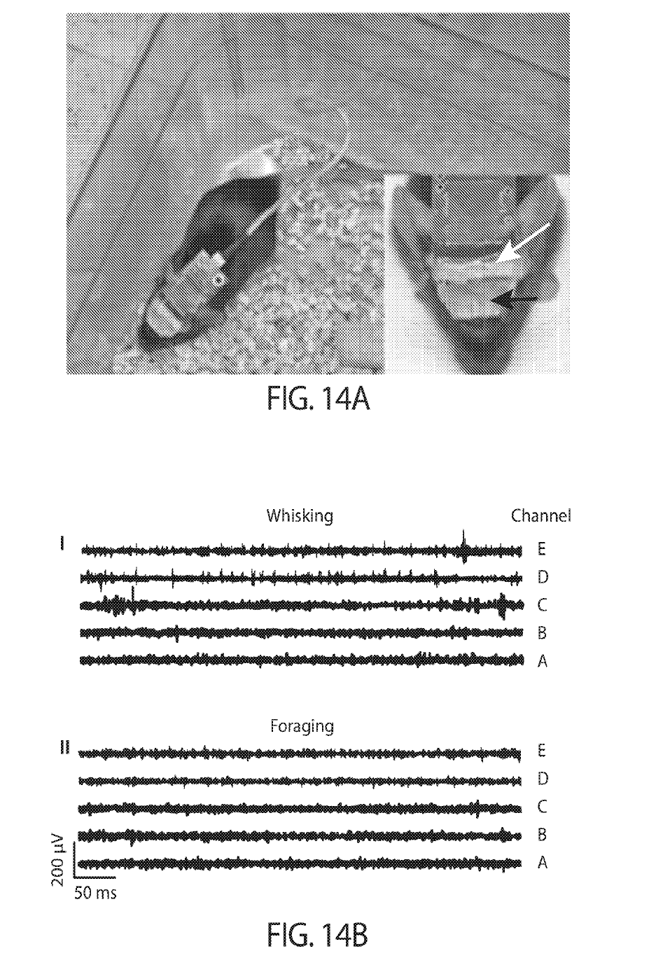

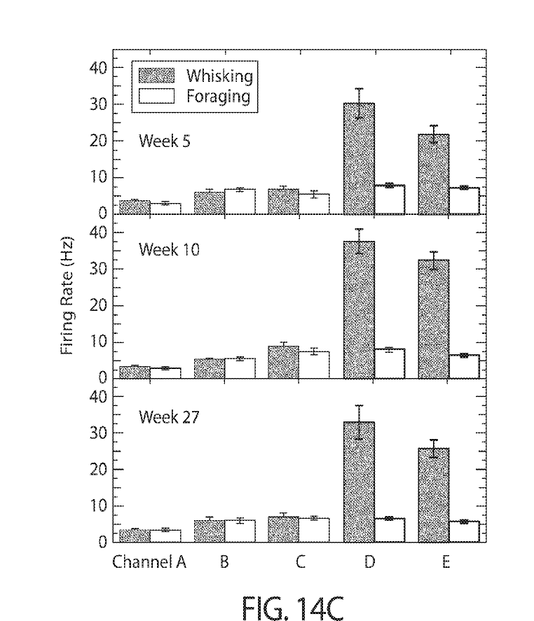

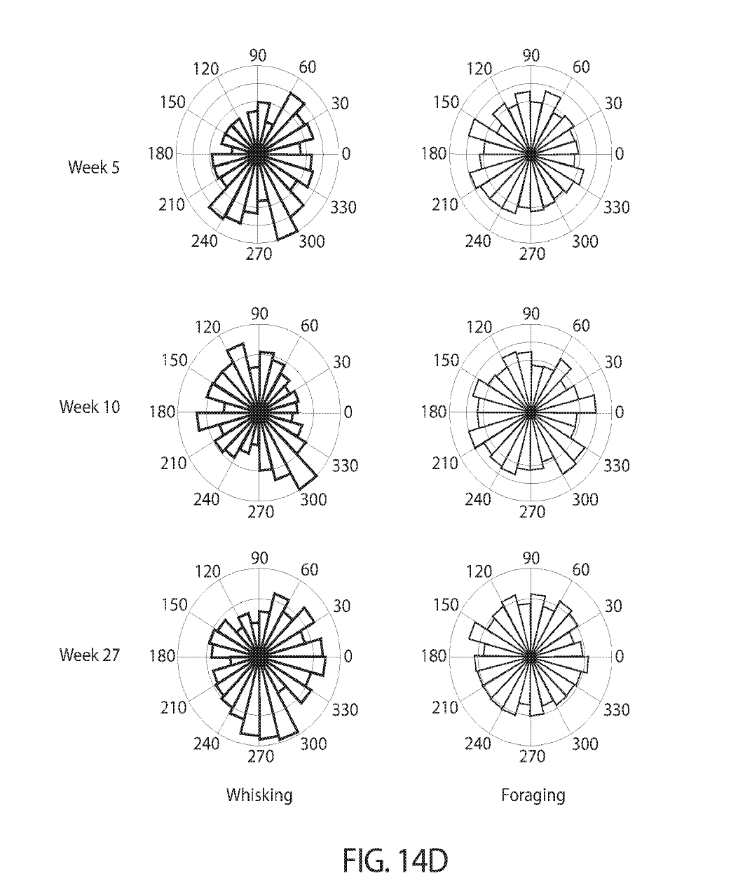

This example shows that mesh electronics were directly bonded to a preamplifier (preamp) connector following injection for chronic studies of freely behaving mice. The lightweight interface was only 1.0 g with the preamp plugged-in (0.35 g without preamp), allowing data acquisition through a highly flexible cable that did not restrict animal motion. The interface had minimal impact on the housed animal without preamp given its low profile and weight. Single-unit recordings from five channels at 5 weeks post-injection (FIG. 14B) were grouped into periods when the mouse was whisking food (I) or foraging (II) in a novel environment. The two channels located in CTX barrel field (Channels D&E) consistently showed behavior-related firing rate increases during whisking, while the other three channels exhibited no significant changes across the 27-week measurement course (FIG. 14C). Analyses of sorted spikes within the same recording session revealed comparable fluctuations in the intrinsic recording noise and stable unit isolation. Interestingly, phase locking analyses between single-unit firings in barrel CTX (Channel D) and theta-band LFP oscillations in HIP (Channel A) at different time points indicated relatively constant locking at ˜300 degrees during active whisking versus no identifiable phase coherence during foraging (FIG. 14D). These findings are consistent with a pathway linking the barrel field that receives vibrissa input and HIP with higher-order processing of texture information.

FIG. 14 shows chronic recordings from a freely behaving mouse. FIG. 14A is a photograph of a typical freely behaving mouse recording. A voltage-amplifier was directly positioned near the mouse head to minimize mechanical noise coupling. A flexible serial peripheral interface (SPI) cable was used to transmit amplified signals to the data acquisition systems. Inset: A zoom-in view showing the conductive ink (black lines), FFC (lower arrow), Omnetics connector (upper arrow) and the voltage-amplifier (rectangle). FIG. 14B shows single-unit spike recordings at 5 weeks post-injection from five representative channels, two of which (Channels D & E, shown in red) are located in the somatosensory CTX, when the mouse was whisking food pellets (I) and foraging in the cage (II). FIG. 14C shows bar charts summarizing the changes in firing rate for the same five channels as shown in FIG. 14B during whisking (black bars) and foraging (white bars) at 5 (top), 10 (middle) and 27 weeks (bottom) post-injection. The two channels (Channels D & E) with whisking-associated neuronal responses are highlighted with red borders. Error bars indicate +/−1 SEM. FIG. 14D shows polar plots showing phase-locking of single-unit spikes recorded in the CTX barrel field (Channel D) to the theta oscillations (4-8 Hz) of LFPs in the HIP (Channel A) when the mouse was whisking (left column) and foraging (right column) at 5, 10 and 27 weeks.

In summary, the chronic in vivo mesh electronics recording/stimulation platform discussed above has achieved stable multiplexed LFP and single-unit spike recordings from mouse HIP and CTX with tracking of the same neurons and neural circuits up to eight-month periods. These results contrast conventional brain probes that generally exhibit spike shape changes over days to weeks. Stable characteristics of the mesh platform also were shown for simultaneous recording from distinct brain regions using multiple mesh implants and for stimulation and recording from neurons. This platform was used for longitudinal studies of aging-associated neuronal changes and long-term recording in freely behaving mice. Consistent and reproducible stable chronic recording/stimulation from the same single neurons/neural circuits has been unattainable previously for more than several weeks.

Mechanistically, these findings correlate with the comparable bending stiffness values for the mesh electronics and neural tissue, which minimizes or eliminates relative shear motion of the electronics inside brain since the implant is effectively decoupled from the I/O fixed to skull. In addition, near natural distributions of neurons, axons and glial cells at the mesh electronics surface and interior shown for more than 6 weeks (FIGS. 10D-10E) contrasts a 50-200 micrometer region of neuron depletion around conventional brain implants, substantiating the observed stable single-unit spike amplitudes for the same time periods.

Moreover, time-dependent histology (FIGS. 10D-10E) and waveform auto-/cross-correlation analyses provide insight into the amplitude increase seen at earlier times (FIG. 10B). First, the histology data suggest that amplitude increase was associated with recovery from acute implantation damage. Second, representative waveform autocorrelation analysis during this period indicates the units identified at 1 week post-injection remained consistent through 8 weeks. The somewhat lower percentage of cross-week autocorrelation for some neurons might suggest a contribution from axon/dendrite regeneration. Last, the observation of a new cluster (neuron 3, FIG. 11B) at 3 weeks indicates that tissue remodeling (also seen in histology data) also contributes.

In addition, the gradually decreasing firing rate, which was found to negatively correlate with progressively increasing peak-to-trough time in individual neurons (FIG. 13), is consistent with impaired long-term potentiation (LTP), decreased [Ca²⁺]ᵢ baseline and increased after-hyperpolarization (AHP) as suggested by cross-sectional rodent studies. Not only are these observations qualitatively consistent with population-averaged results discovered by cross-sectional studies, but they also reveal details on the evolution of individual neurons during aging that has been previously inaccessible.

Last, it may be beneficial to increase the number of recording channels and to achieve full-amplitude spikes closer to initial implantation for our mesh platform. A combination of increasing the number/density of electrodes in each mesh probe and multi-site injection of several meshes into the same animal provides a feasible strategy for achieving higher multiplexing. The unique capability to record from and stimulate the same neurons and neural circuits over at least eight-month periods opens up important neurobiology opportunities, including understanding fundamental neural circuit plasticity, reorganization and development during learning, memory formation and aging-associated cognitive decline, as well as enabling closed-loop BMIs in freely behaving animals via stable single-neuron based decoding and communication.

### EXAMPLE 14

Fabrication of syringe-injectable electronics. The syringe-injectable mesh electronics for chronic brain activity mapping used fabrication procedures and geometrical designs as discussed above. Key mesh parameters were as follows: total mesh width, W=2 mm, longitudinal SU-8 ribbon width, w₁=20 micrometers, transverse SU-8 ribbon width, w₂=20 micrometers, angle between longitudinal and transverse SU-8 ribbons, alpha=45°, longitudinal spacing (pitch between transverse ribbons), L₁=333 micrometers, transverse spacing (pitch between longitudinal ribbons), L₂=125 micrometers, metal interconnect line width, wₘ=10 micrometers and total number of recording channels, N=16. Fabrication steps are as follows: (i) A sacrificial layer of Ni with a thickness of 100 nm was thermally evaporated (Sharon Vacuum, Brockton, Mass.) onto a 3 inch Si wafer (n-type 0.005 Ohm cm, 600-nm thermal oxide, Nova Electronic Materials, Flower Mound, Tex.), which was pre-cleaned with oxygen plasma. (ii) Negative photoresist SU-8 (SU-8 2000.5; MicroChem Corp., Newton, Mass.) was spin-coated on the Si wafer to a thickness of 500 nm, pre-baked sequentially at 65° C. for 1 min and 95° C. for 4 min, and then patterned by photolithography (PL) with a mask aligner (ABM mask aligner, San Jose, Calif.). After PL exposure the sample was post-baked sequentially at 65° C. for 3 min and 95° C. for 3 min. (iii) The SU-8 photoresist was then developed (SU-8 Developer, MicroChem Corp., Newton, Mass.) for 2 min, rinsed with isopropanol, dried in a N₂ flow and hard-baked at 185° C. for 1 h. (iv) The wafer was then cleaned with oxygen plasma (50 W, 1 min), spin-coated with MCC Primer 80/20 and LOR 3A lift-off resist (MicroChem Corp., Newton, Mass.), baked at 185° C. for 5 min, followed by spin-coating Shipley 1805 positive photoresist (Microposit, The Dow Chemical Company, Marlborough, Mass.), which was then baked at 115° C. for 5 min. The positive photoresist was patterned by PL and developed (MF-CD-26, Microposit, The Dow Chemical Company, Marlborough, Mass.) for 90 s. (v) A 1.5-nm thick Cr layer and a 100-nm thick Au layer were sequentially deposited by electron-beam evaporation (Denton Vacuum, Moorestown, N.J.), followed by a lift-off step (Remover PG, MicroChem Corp., Newton, Mass.) to make the Au interconnect lines. (vi) Steps iv and v were repeated for PL patterning and deposition of the Pt sensing or stimulation electrodes (Cr: 1.5 nm, Pt: 50 nm). The diameter of Pt sensing electrodes was 20 micrometers and that of Pt stimulation electrode was increased to 150 micrometers (the larger diameter was used to afford lower impedance for electrical stimulation). (vii) Steps ii and iii were repeated for PL patterning of the top SU-8 layer, which served as the top encapsulating/insulating layer of the metal interconnect lines. (viii) Subsequently, the Si wafer was cleaned with oxygen plasma (50 W, 1 min) and then transferred to a Ni etchant solution comprising 40% FeCl₃:39% HCl:H₂O=1:1:20 to remove the sacrificial Ni layer and release the mesh electronics from the Si substrate. Released mesh electronics were rinsed with deionized (DI) water, transferred to an aqueous solution of poly-D-lysine (PDL, 1.0 mg/ml, MW 70,000-150,000, Sigma-Aldrich Corp., St. Louis, Mo.) for 24 h, and then transferred to 1× phosphate buffered saline (PBS) solution (HyClone™ Phosphate Buffered Saline, Thermo Fisher Scientific Inc., Pittsburgh, Pa.) before use.

Vertebrate animal subjects. Adult (25-35 g) male C57BL/6J mice (Jackson Laboratory, Bar Harbor, Me.) were the vertebrate animal subjects used in this study. The total number of mice used for demonstrating chronic single neuron level recordings is 4, which was statistically determined by power analysis by assuming a significance level of 5% and an average spike amplitude to variation ratio of 3.0 at 90% power. Moreover, a 5^{th} mouse with two meshes injected was used to show multi-site injection stability, a 6^{th} subject was used for stimulation studies, and a 7^{th} mouse was used for freely behaving mouse recordings, and 3 additional mice were used for immunohistochemical studies. Exclusion criteria were pre-established: animals with failed surgery or substantial acute implantation damage (>100 microliters of initial liquid injection volume) were discarded from further chronic recordings. Randomization or blinding study was not applicable to this study. All procedures performed on the mice were approved by the Animal Care and Use Committee of Harvard University. The animal care and use programs at Harvard University meet the requirements of the Federal Law (89-544 and 91-579) and NIH regulations and are also accredited by the American Association for Accreditation of Laboratory Animal Care (AAALAC). Animals were group-housed on a 12 h:12 h light:dark cycle in the Harvard University's Biology Research Infrastructure (BRI) and fed with food and water ad libitum as appropriate.

In vivo mouse survival surgery. Stereotaxic injection of mesh electronics in mouse brain. In vivo injection of mesh electronics into the brains of live mice was performed using a controlled stereotaxic injection method. First, all metal tools in direct contact with the surgical subject were autoclaved for 1 h before use, and all plastic tools in direct contact with the surgical subjects were sterilized with 70% ethanol and rinsed with sterile DI water and sterile 1× PBS before use. Prior to injection, the mesh electronics were sterilized with 70% ethanol followed by rinsing in sterile DI water and transfer to sterile lx PBS. The mesh was loaded into glass capillary needles.

C57BL/6J mice were anesthetized by intraperitoneal injection of a mixture of 75 mg/kg of ketamine (Patterson Veterinary Supply Inc., Chicago, Ill.) and 1 mg/kg dexdomitor (Orion Corporation, Espoo, Finland). The degree of anesthesia was verified via the toe pinch method before the surgery started. To maintain the body temperature and prevent hypothermia of the surgical subject, a homeothermic blanket (Harvard Apparatus, Holliston, Mass.) was set to 37° C. and placed underneath the anesthetized mouse, which was placed in the stereotaxic frame (Lab Standard Stereotaxic Instrument, Stoelting Co., Wood Dale, Ill.) equipped with two ear bars and one nose clamp that fixed the mouse head in position. Puralube ocular lubricant (Dechra Pharmaceuticals, Northwich, UK) was applied on both eyes of the mouse to moisturize the eye surface throughout the surgery. Hair removal lotion (Nair®, Church & Dwight, Ewing, N.J.) was used for depilation of the mouse head and iodophor was applied to sterilize the depilated scalp skin. A 1-mm longitudinal incision along the sagittal sinus was made in the scalp with a sterile scalpel, and the scalp skin was resected to expose a 6 mm×8 mm portion of the skull. METABOND® enamel etchant gel (Parkell Inc., Edgewood, N.Y.) was applied over the exposed cranial bone to prepare the surface for mounting the electronics on the mouse skull later.

A 1 mm diameter burr hole was drilled using a dental drill (Micromotor with On/Off Pedal 110/220, Grobet USA, Carlstadt, N.J.) according to the following stereotaxic coordinates: anteroposterior: −4.96 mm, mediolateral: 3.10 mm. After the hole was drilled, the dura was carefully incised and resected using a 27-gauge needle (PrecisionGlide®, Becton Dickinson and Company, Franklin Lakes, N.J.). Then a sterilized 0-80 set screw (18-8 Stainless Steel Cup Point Set Screw; outer diameter: 0.060 inch or 1.52 mm, groove diameter: 0.045 inch or 1.14 mm, length: 3/16 inch or 4.76 mm; McMaster-Carr Supply Company, Elmhurst, Ill.) was screwed into this 1-mm burr hole to a depth of 500 micrometers as the grounding and reference electrode. Another 1 mm burr hole was drilled for injection of mesh electronics according to the following stereotaxic coordinates depending on specific brain areas for activity recording:

1) Primary somatosensory cortex, barrel field: anteroposterior: −1.82 mm, mediolateral: −3.00 mm, dorsoventral: 0.75 mm.

2) Primary somatosensory cortex, trunk: anteroposterior: −1.70 mm, mediolateral: −2.00 mm, dorsoventral: 0.75 mm.

3) Hippocampal CA1 field: anteroposterior: −1.70 mm, mediolateral: −1.60 mm, dorsoventral: 1.17 mm.

4) Hippocampal CA3 field: anteroposterior: −1.70 mm, mediolateral: −2.00 mm, dorsoventral: 1.85 mm.

The dura was removed from the burr hole drilled for mesh electronics injection and sterile 1× PBS was swabbed on the surface of the brain to keep it moist throughout the surgery. The mesh electronics was injected into the desired brain region using a controlled injection method. In brief, the mesh electronics was loaded into a glass capillary needle with inner diameter (I.D.) of 400 micrometers and outer diameter (O.D.) of 650 micrometers (Produstrial LLC, Fredon, N.J.). The glass capillary needle loaded with mesh electronics was mounted onto the stereotaxic stage through a micropipette holder (Q series holder, Harvard Apparatus, Holliston, Mass.), which was connected to a 5 mL syringe (Becton Dickinson and Company, Franklin Lakes, N.J.) through a polyethylene Intramedic™ catheter tubing (I.D. 1.19 mm, O.D. 1.70 mm). Controlled injection was achieved by balancing the volumetric flow rate (typically 20-50 mL/h), which was controlled by a syringe pump (PHD 2000, Harvard Apparatus, Holliston, Mass.), and the needle withdrawal speed (typically 0.2-0.5 mm/s), which was controlled by a motorized linear translation stage (860A motorizer and 460A linear stage, Newport Corporation, Irvine, Calif.). Using the controlled injection method with field of view (FoV) visualization through an eyepiece camera (DCC1240C, Thorlabs Inc., Newton, N.J.), the mesh electronics was delivered to specific brain regions with elongated morphology along the injection direction with ˜20 micrometers spatial targeting precision. For successful long-term recordings, the total injection volume is usually between 10 and 100 microliters. An unexpected large injection volume (>100 microliters) could result in brain edema or failure of recovery from acute surgical damage, leading to expulsion of the subject from the study.

Electrical connection of syringe-injectable electronics for chronic recordings from awake and restrained mice. After the injection of mesh electronics into the desired region of a mouse brain, the stereotaxic stage was moved to reposition the glass capillary needle over a 16-channel flexible flat cable (FFC, PREMO-FLEX, Molex Incorporated, Lisle, Ill.), and then the remaining mesh electronics was fully expelled from the needle and unfolded onto the FFC to expose the input/output (I/O) connection pads. High-yield bonding of mesh electronics I/O pads to the FFC was carried out using a conductive ink printing method. In brief, the print head loaded with carbon nanotube solution (Stock No.: P093099-11, Tubes@Rice, Houston, Tex.) was driven by a motorized micromanipulator (MP-285/M, Sutter Instrument, Novato, Calif.) through a user-written LabVIEW program to print conductive ink automatically and connect each mesh I/O pad to each of the FFC lines to enable independently addressable sensor elements. Failure of mesh I/O unfolding could lead to potential low-yield electrical connection to the FFC interface cable. All printed conductive lines were passivated by METABOND® dental cement (Parkell Inc., Edgewood, N.Y.), and then the entire FFC with mesh electronics bonded to the FFC was cemented to the mouse skull with METABOND® dental cement. The FFC was folded to reduce its size on the mouse skull. The total mass of the bonded interface cable with mesh electronics is typically 0.2 to 0.3 g.

Electrical connection of syringe-injectable electronics for chronic recordings from freely behaving mice. After the injection of mesh electronics into the desired mouse brain region, the stereotaxic stage was manually moved to reposition the glass capillary needle to a 32-channel Omnetics male connector (A79024-001, Omnetics Connector Corp., Minneapolis, Minn.) with a weight of ˜0.1 g glued on a nonconductive polyethylene terephthalate (PET) flexible substrate with a thickness of ˜0.3 mm, and then the remaining mesh electronics was fully expelled from the needle and unfolded onto the flexible substrate with its I/O connection pads facing the horizontal mounting tails of the Omnetics connector. Conductive ink printing was used to bond the mesh electronics I/O pads to 16 horizontal mounting tails of the Omnetics connector as described above for the FFC cable. The 0-80 grounding screw was electrically connected to one of the four pre-installed grounding/reference pins of the Omnetics connector using silver conductive epoxy (MG Chemicals, Burlington, ON, Canada). All printed conductive lines were protected by METABOND® dental cement, before the entire packaged headstage was cemented to the mouse skull with dental cement.

Postoperative care. After surgery was complete, antibiotic ointment (WATER-JEL Technologies LLC, Carlstadt, N.J.) was applied copiously around the wound, and the mouse was returned to the cage equipped with a 37° C. heating pad and its activity monitored every hour until fully recovered from anesthesia (i.e., exhibiting sternal recumbency and purposeful movement). Buprenex (Buprenorphine, Patterson Veterinary Supply Inc, Chicago, Ill.) analgesia was given intraperitoneally at a dose of 0.05 mg/kg body weight every 12 h for up to 72 h post brain surgery.

The overall success rate of the surgical procedure was around 70%, with the main causes for failure including (i) an unexpected large injection volume (>100 microliters) resulting in brain edema, and (ii) failure of mesh I/O unfolding leading to low-yield electrical connection to the FFC interface cable. Further improvements on surgery success rate included: (i) better control of injection volume by further reducing the transverse bending stiffness of mesh electronics; and (ii) more reliable I/O unfolding/bonding through designs of I/O pads distributions with larger separation.

Micro-Computed Tomography. One mouse injected with mesh electronics, where the I/O was bonded to an FFC and then cemented to the mouse skull, was euthanized via intraperitoneal injection of Euthasol at a dose of 270 mg/kg body weight and decapitated. The decapitated mouse head was imaged using an HMXST Micro-CT X-ray scanning system with a standard horizontal imaging axis cabinet (model: HMXST225, Nikon Metrology, Inc., Brighton, Mich.). Imaging parameters were set as 115 kV and 83 microamps (with a 0.1-mm copper filter for beam hardening) for scanning the decapitated mouse head. Before scanning, shading correction and flux normalization were applied to adjust the X-ray detector. The CT Pro 3D software (ver. 2.2, Nikon-Metris, UK) was used to calibrate centers of rotation for micro-CT sinograms and to reconstruct all 2D images. VGStudio MAX software (ver. 2.2, Volume Graphics GMbh, Germany) was used for 3D rendering and analysis of the reconstructed images.

In vivo chronic brain recording and stimulation in mice. Chronic brain recording from awake and restrained mice. Mice with implanted mesh electronics and FFC connector were recorded chronically on a weekly basis, starting from Day 7 post-injection and surgery. Mice were restrained in a Tailveiner® restrainer (Braintree Scientific LLC., Braintree, Mass.) while its head-mounted FFC was connected to an Intan RHD 2132 amplifier evaluation system (Intan Technologies LLC., Los Angeles, Calif.) through a home-made printed circuit board (PCB). The 0-80 set screw was used as a reference. Electrophysiological recording was made with a 20-kHz sampling rate and a 60-Hz notch filter, while the electrical impedance at 1 kHz of each recording electrode was also measured by the same Intan system.

Chronic brain recording of freely behaving mice. Mice with Omnetics connectors were recorded chronically on a weekly basis when they were freely roaming in the cage. For recording, an Intan preamplifier chip (RHD2132 16-Channel Amplifier Board, Intan Technologies LLC., Los Angeles, Calif.) with pre-installed female Omnetics connector was connected directly to the male Omnetics connector cemented on the mouse skull during surgery, and the mouse was allowed to roam in a cage environment not explored previously. Food pellets were placed at random positions inside the cage for each trial. Electrophysiological recordings were made using the same Intan evaluation system with a 20-kHz sampling rate and a 60-Hz notch filter, and were synchronized with video recording of the mouse's motion inside the cage using a digital camera.

Chronic electrical stimulation of mouse brains. Mice injected with mesh electronics incorporating stimulation and recording electrodes were subject to electrical stimulation and simultaneous electrophysiological recording periodically to week 14 post-surgery. Similar to chronic electrophysiological recording described above, mice were restrained in the Tailveiner® restrainer with a head-mounted FFC connected to the Intan RHD 2132 amplifier evaluation system for 12 recording channels, while the other 4 stimulation channels were connected to a homemade stimulator comprising a function generator (Model 33220A, 20 MHz Function/Arbitrary Waveform Generator, Agilent Technologies, Santa Clara, Calif.) that provided stimulus pulse trains with user-defined current, pulse duration and pulse interval. Typical currents used for stimulation ranged from 5-50 microamps, followed by an inverted polarity with the same amplitude to provide capacitor-coupled and charge-balanced stimulation. The pulse duration was 1 ms for each phase (positive or negative) with two consecutive pulses spaced by 1 s. Neural responses to stimulus input through one of the 4 stimulation electrodes were recorded as both local field potentials (LFPs) and single-unit spikes from the 12 recording electrodes from the same injected mesh electronics. The 0-80 set screw was used as a reference for both stimulation and recording.

Data analysis of electrophysiological recording. Data analysis of LFP and single-unit action potential recording. The electrophysiological recording data was analyzed offline. In brief, raw recording data was filtered using non-causal Butterworth bandpass filters (‘filtfilt’ function in Matlab) in the 250-6000 Hz frequency range to extract single-unit spikes, in the 0.1-150 Hz range to extract LFP, and in the 4-8 Hz range to extract the theta rhythm of LFP. The intrinsic noise distribution of a specific channel was analyzed based on all recording traces bandpass filtered at 250-6000 Hz excluding any firing spikes. The correlation coefficient maps of single-unit spike recording traces were calculated based on the standard Pearson product-moment correlation coefficient for time series. Namely, for two spike traces, Y₁ (t) and Y₂ (t), the correlation coefficient between them is calculated as:

$$Corr ( {Y}_{1} , {Y}_{2} ) = \frac{{\int }_{{T}_{1}}^{{T}_{2}} ( {Y}_{1} ( t ) - {\overline{Y}}_{1} ) ( {Y}_{2} ( t ) - {\overline{Y}}_{2} ) dt}{\sqrt{{\int }_{{T}_{1}}^{{T}_{2}} {( {Y}_{1} ( t ) - {\overline{Y}}_{1} )}^{2} {( {Y}_{2} ( t ) - {\overline{Y}}_{2} )}^{2} dt}} \qquad ( 1 )$$

where T₁ and T₂ indicate the starting and ending time of the recording traces, and

$${\overline{Y}}_{i} = {\int }_{{T}_{1}}^{{T}_{2}} {Y}_{i} ( t ) dt / ( {T}_{2} - {T}_{1} ) ( i = 1 , 2 )$$

represents the averaged value of Yᵢ (t) over the time period between T₁ and T₂.

Single-unit spike sorting was performed by amplitude thresholding of the filtered traces by automatically determining the threshold based on the median of the background noise according to the improved noise estimation method. The average spike amplitude for each recording channel was defined as the peak-to-peak amplitude of the spikes for a typical 1-min recording trace. All of the single-neuron spike analyses shown in FIG. 11 were carried out based on a 30-min recording session. The peak-to-trough time for each recorded spike was defined as the time interval τ (tau) between the major peak (which can be either positive or negative) and the following rebound with opposite polarity (FIG. 13A, II, upper left inset). All sorted spikes were clustered to determine the number of single neurons and assign spikes to each single neuron using the WaveClus software that employs unsupervised superparamagnetic clustering of single-unit spikes. Spikes assigned to the same cluster were coded with the same color and plotted in the first and second principal components (PC1-PC2) plane. The noise distribution of sorted spikes was obtained by plotting the histogram of the difference between each raw spike and average spike waveform at every sampling point. The deviations of all the identified neuron noise distributions were computed by fitting each noise histogram to a Gaussian distribution.

The L-ratio for each cluster of spikes was calculated as follows

$${L}_{ratio} = \frac{L ( C )}{N ( C )} = \frac{{\sum }_{i ∉ C} 1 - {CDF}_{{χ}^{2}} ( {D}_{i , C}^{2} )}{N ( C )} \qquad ( 2 )$$

where N(C) denotes the total number of spikes in the cluster, CDFχ² presents the cumulative distribution function of the χ² distribution in an eight-dimensional feature space, and D²_{i,C} is the Mahalanobis distance of a spike i from the center of the cluster C. The summation goes over the entire set of spikes that do not belong to the cluster. An L-ratio of <0.05 is generally considered good cluster separation/isolation.

The autocorrelation and cross-correlation of raw and average spike waveforms were computed based on the standard Pearson product-moment correlation coefficient defined in Equation (1) for the 3 ms time series of each spike. A value of 1 indicates identical spike shapes, irrespective of absolute spike amplitudes.

The spiking times of all clustered single-unit action potentials assigned to each cluster (i.e., each single neuron) were used to compute the interspike interval (ISI) histogram under different bin sizes for verification of unit isolation and extraction of firing rate by fitting the ISI histogram to a first order exponential decay (FIG. 11C, bin size=20 ms). The instantaneous phase of the theta rhythm of LFP at the location of each single-unit spike that had been assigned to a certain cluster was determined by performing Hilbert transform of the filtered traces in the 4-8 Hz frequency range and phase locking behavior of single-unit spikes was investigated by plotting their phase distribution in a polar plot. All the extracted phases of individual spikes with respect to theta rhythm LFP in each recording session were subjected to a Rayleigh Z-test, and Ln(Z) values obtained from multiple recording sessions (across different weeks) for each identified neuron were used to test the statistical significance of each neuron's phase-locking behavior. A subsequent Rayleigh Z test was then applied to the extracted locked phases from each week's phase distribution from 3 to 34 weeks post-injection to test the chronic stability of each neuron's phase-locking behavior.

Data analysis of freely behaving recording. For freely behaving mice, the raw recording data was taken synchronously with mouse video recording, and then bandpass filtered before single-unit spikes were sorted and clustered as described above. The video of mouse movement was analyzed with Gaussian blur filter and object tracking algorithm using Matlab to extract the mouse's trajectory and the distance between its head and the food pellet in real time. The firing rate of the electrophysiological recording was then correlated with the mouse's motion trajectory to derive the interaction-dependent firing behavior when the mouse whisked food pellets in its environment. Phase-locking analyses were performed using the same algorithm described above between the single-unit spikes from Channel D (located in the barrel field of somatosensory cortex) and the theta rhythm of LFP from Channel A (located in hippocampus) shown in FIG. 14 for data recorded when the mouse was whisking food pellets and foraging. These phase-locking results are presented separately for durations of active whisking and non-tactile foraging based on dynamic image processing of the video recording the mouse movement in the cage.

Data analysis of electrical stimulus provoked recording. For analysis of recording data with electrical stimulation, the onset time of each stimulus was determined by the large artifact peak due to stimulation input picked by all recording electrodes. All stimulation trials were aligned to that peak as t=0 s (where t is the peristimulus time, t<0 denotes before stimulation and t>0 denotes after stimulation), based on which peristimulus raster plot and post-stimulus first spike latency histogram were plotted using Matlab.

Chronic immunohistochemistry. Histology sample preparation. Mice with implanted mesh electronics at post-injection times of 2, 6 and 12 weeks were anesthetized with ketamine and dexdomitor, and then were transcardially perfused with 40 mL 1× PBS and 40 mL 4% formaldehyde (Sigma-Aldrich Corp., St. Louis, Mo.), followed by decapitation. The scalp skin was removed and the exposed skull was ground for 10-20 min at 10,000 RPM using a high-speed rotary tool (Dremel, Mount Prospect, Ill.). The brain was resected from the cranium and placed in 4% formaldehyde for 24 h, and then transferred to lx PBS for another 24 hours at 4° C. to remove remaining formaldehyde. The brain was transferred to incrementally increasing sucrose solutions (10-30%) (Sigma-Aldrich Corp., St. Louis, Mo.) at 4° C. to cryoprotect the tissue, transferred to cryo-OCT compound (Tissue-Tek® O.C.T. Compound, VWR, Radnor, Pa.) and then frozen at −80° C. The frozen sample was then sectioned into 10-micrometer-thick horizontal slices using Leica CM1950 cryosectioning instrument (Leica Microsystems, Buffalo Grove, Ill.).

Immunohistochemical Staining and Microscopic Imaging. The brain tissue sections were rinsed three times in lx PBS and blocked in a solution of 0.3% Triton X-100 (Life technologies, Carlsbad, Calif.) and 5% goat serum (Life Technologies, Carlsbad, Calif.) in 1× PBS for 1 h at room temperature. Slices were then incubated with the primary antibodies, rabbit anti-NeuN (1:200 dilution, Abcam, Cambridge, UK), mouse anti-Neurofilament (1:400 dilution, Abcam, Cambridge, UK), rat anti-GFAP (1:500 dilution, Thermo Fisher Scientific Inc, Cambridge, Mass.) or rabbit anti-Iba1 (1:250 dilution, Abcam, Cambridge, UK) containing 0.3% Triton X-100 and 3% goat serum overnight at 4° C. NeuN is a neuron-specific nuclear protein, and stains the neural somata. Neurofilament is intermediate filaments found in neurons, and stains neural axons. GFAP is glial fibrillary acidic protein, and stains astrocytes. Iba-1 is a 17-kDa EF hand protein that is specifically expressed in macrophages/microglia, and is up-regulated by the activation of these cells. After incubation, slices were rinsed 9 times for a total of 40 min with 1× PBS, before they were incubated with the secondary antibodies, Alexa Fluor® 488 goat anti-rabbit (1:200 dilution, Abcam, Cambridge, UK), Alexa Fluor® 568 goat anti-mouse (1:200 dilution, Abcam, Cambridge, UK), or Alexa Fluor® 647 goat anti-rat (1:200 dilution, Abcam, Cambridge, UK) for 1 h at room temperature; the specific choices of secondary antibodies were made based on primary antibodies used to stain a given slice. Slices were rinsed 9 times for a total of 30 min after incubation with secondary antibodies, before they were mounted on glass slides with coverslips using ProLong® Gold Antifade Mountant (Life Technologies, Carlsbad, Calif.). The slides remained in dark at room temperature for at least 24 h before microscopic imaging.

Confocal fluorescence imaging of the samples was acquired on a Zeiss LSM 880 confocal microscope (Carl Zeiss Microscopy GmbH, Jena, Germany). Confocal images were acquired using 488 nm, 561 nm and 633 nm lasers as the excitation sources for Alexa Fluor® 488, Alexa Fluor® 568 and Alexa Fluor® 647, respectively. ImageJ software was used for image analysis. The mesh electronics in each slice was imaged with differential interference contrast (DIC) on the same microscope, and is shown with false color in the composite images of FIG. 10D. Fluorescence intensities of Neurofilament, NeuN and GFAP were based on the analysis of zoomed-out images of those shown in FIG. 10D with a field of view of 1.2 mm×1.2 mm. iba-1 results were based on analysis of various brain slices. The fluorescence intensities were normalized (value=1.0, gray dashed horizontal lines in FIG. 10E) against the background values 500 micrometers away from the probe interface for each sample.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the word “about” is used herein in reference to a number, it should be understood that still another embodiment of the invention includes that number not modified by the presence of the word “about.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A method, comprising:
inserting a tube comprising a device comprising one or more nanoscale sensing elements into a medium; and
withdrawing the tube from the medium while urging the device out of the tube, wherein the rate of withdrawal of the tube from the medium is substantially equal to the rate that the device is urged out of the tube.

2-3. (canceled)

4. The method of claim 3, wherein the medium is a brain.

5-8. (canceled)

9. The method of claim 1, wherein the medium is part of a living subject.

10. (canceled)

11. The method of claim 1, wherein urging the device out of the tube comprises expelling fluid through the tube.

12. The method of claim 1, wherein the device comprises a mesh comprising a plurality of nanoscale sensing elements.

13. (canceled)

14. The method of claim 1, wherein the device comprises a biocompatible material.

15. The method of claim 1, wherein the device comprises an extracellular matrix material.

16. The method of claim 1, further comprising passing cells through the tube.

17. (canceled)

18. The method of claim 1, further comprising attaching at least a portion of the device to an electrical circuit external of the device.

19-20. (canceled)

21. The method of claim 1, wherein at least 50% of the nanoscale sensing elements within the device form portions of one or more electrical circuits connectable to one or more electrical circuits that are external of the device.

22. The method of claim 1, wherein the device an electrical network comprising at least some of the nanoscale sensing elements.

23. The method of claim 22, wherein the electrical network is formed from a curled and/or folded two-dimensional structure.

24-40. (canceled)

41. The method of claim 1, wherein at least about 50% of the nanoscale sensing elements within the device are individually electronically addressable.

42. A method, comprising:
inserting a tube comprising a device comprising one or more nanoscale sensing elements into a medium; and
removing the tube from the medium, without substantially altering the position of the device relative to the medium.

43-44. (canceled)

45. The method of claim 44, wherein removing the tube comprises withdrawing the tube from the medium while urging the device out of the tube.

46. (canceled)

47. The method of claim 42, wherein removing the tube comprises dissolving the tube.

48-83. (canceled)

84. A method, comprising:
inserting a device comprising one or more nanoscale sensing elements into a medium;
connecting the device to an electrical interface by printing a conductive path directly onto the surface of the medium, wherein the conductive path is in electrical communication with the one or more nanoscale sensing elements; and
covering at least a portion of the conductive path with an insulating material.

85. (canceled)

86. The method of claim 84, wherein the conductive path comprises carbon nanotubes.

87-92. (canceled)

93. The method of claim 84, wherein the conductive path is printed using a print head controlled by a micromanipulator.

94. (canceled)

95. The method of claim 84, wherein the insulating material comprises an elastomer.

96. (canceled)
